(19) United States
(12) Patent Application Publication
(10) Pub. No.: US 2026/0138133 A1
(43) Pub. Date: May 21, 2026

(54) PARTICLE SEPARATOR SYSTEM, MATERIALS, AND METHODS OF USE

(71) Applicant: LEVITASBIO, INC., Menlo Park, CA (US)

(72) Inventors: Seth BRODIE, Belmont, CA (US); Susana JETT, Newark, CA (US); Kevin TRAVERS, Menlo Park, CA (US)

(21) Appl. No.: 19/333,278

(22) Filed: Sep. 18, 2025

Related U.S. Application Data
(63) Continuation-in-part of application No. 19/203,107, filed on May 8, 2025.
Application No. 19/203,107 is a continuation of application No. PCT/US2024/019795, filed on Mar. 13, 2024.
(60) Provisional application No. 63/489,985, filed on Mar. 13, 2023.

(51) Int. Cl. B01L 3/00; B01D 21/00; B82Y 15/00; C12N 5/00; G01N 35/00; G01N 35/04

(52) U.S. Cl. CPC B01L 3/502761; B01D 21/0075; C12N 5/0081; C12N 5/522; C12N 5/56; G01N 35/0099; B01L 2200/0652; B01L 2300/0663; B01L 2300/0829; B01L 2300/0877; B01L 2300/0883; B01L 2300/168; B01L 2400/043; B82Y 15/00; G01N 2035/0434

(57) ABSTRACT

The present invention relates generally to the concentration of particulate containing samples, such as cells or biomolecules, in order to isolate such particles within a medium and to isolate particle depleted medium. In some embodiments, the present invention relates generally to the separation and/or concentration of cell nuclei from nuclear debris and dead cells.

### CROSS-REFERENCE TO THE RELATED APPLICATIONS

This application claims priority to U.S. application Ser. No. 19/203,107, filed May 8, 2025, which is a continuation of International Application No. PCT/US2024/019795, filed Mar. 13, 2024, which claim priority benefit of U.S. Provisional Application Ser. No. 63/489,985, filed on Mar. 13, 2023, the contents of each of which are incorporated herein by reference in their entireties.

### TECHNICAL FIELD OF THE INVENTION

The present invention relates generally to the concentration of particulate containing samples, such as cells or biomolecules, in order to isolate such particles within a medium and to isolate particle depleted medium. In some embodiments, the present invention relates generally to the separation and/or concentration of cell nuclei from nuclear debris and dead cells.

### BACKGROUND OF THE INVENTION

Isolation of particles contained within a medium is an important step in many chemical and biological processes. In some processes there may be a need to simply isolate a particle to facilitate the use or manipulation of the particle, whereas in other processes there may be a need to separate the particle from other particles that are also present in the medium. Various devices have been developed to facilitate such particle isolation and separation. In addition, there have been attempts to develop devices that rely on the magnetic properties of the particles and their surrounding medium in order to separate out particles of interest from heterogenous populations of particles.

A common need when working with cells is to concentrate the cells by reducing the volume that the cells are suspended in. The most common procedure for cell concentration is to centrifuge the cells to form a pellet and removing a large portion of the media. Centrifugation involves the application of centrifugal force to separate particles from a solution according to their size, shape, density, viscosity of the medium, and rotor speed. However, there are instances in which centrifugation is undesirable, where centrifugation can create damage to the cells or activate the cells. For example, centrifugation with T cells can lead to activation of the cells. Additionally, when working with rare or low volume samples, bulk separation techniques such as centrifugation can be extraordinarily wasteful or laborious and do not easily allow for fractionation of the sample. Also, when the particles to be separated are fragile or labile, such as when working with biological entities, precise conditions to enhance particle stability can be challenging.

Nuclei have quickly become one of the main targets for single-cell research and development when working with difficult to process tissues and samples. While working with cells should be much simpler and more direct than nuclei, the realities of processing tissues are quite often much more complex and difficult than expected.

Tissues vary in their cellular composition, extracellular matrix, and dissociation behavior when processed. As such researchers have found it increasingly difficult to establish well-validated and uniformly performing dissociation and purification protocols that produce a reliable, high-quality single-cell suspension. Alternatively, nuclei workflows overcome many of these challenges by not only providing a much more uniform and standardized starting material for single-cell analysis but also remove some of the potentials for gene expression changes associated with tissue dissociation and processing.

Unfortunately, nuclei preparation comes with its own challenges. Specifically, how to purify extracted nuclei away from dead cells and debris. Traditional methods continue to be difficult and time-consuming to use, producing poor yields, and purities. The LeviCell offers a fast, simple, and highly efficient method to purify nuclei.

The devices and methods described herein address these issues by providing alternative methods for concentrating particles, e.g. cells and cell nuclei, and producing particle-depleted medium that does not depend on the high mechanical forces that are required during centrifugation.

### SUMMARY OF THE INVENTION

The inventive embodiments provided in this Summary of the Invention are meant to be illustrative only and to provide an overview of selected embodiments disclosed herein. The Summary of the Invention, being illustrative and selective, does not limit the scope of any claim, does not provide the entire scope of inventive embodiments disclosed or contemplated herein, and should not be construed as limiting or constraining the scope of this disclosure or any claimed inventive embodiment.

Provided herein is a method (“Method 1”) for extracting cellular nuclei from intact cells comprising; providing intact cells; and lysing the cells in a nuclei isolating buffer; wherein the nuclei isolating buffer comprises wheat germ agglutinin (WGA).

Provided herein is a Method (“Method 1a”) of reducing contamination in isolated cell nuclei from ambient RNA, comprising isolating the cell nuclei in the presence of wheat germ agglutinin (WGA).

Provided herein is a method (“Method 2”) of isolation of a target subcellular component, e.g. cellular nuclei, from a sample comprising the target subcellular component and one or more contaminating species, comprising:
loading a sample comprising the target subcellular component, the contaminating species, and a sample medium comprising:
   i) a paramagnetic compound or ferrofluid; and
   ii) isolation particles (or beads);
      into a separation well or channel along which the sample is optionally caused to flow;
subjecting the sample to a magnetic force with at least one magnet to affect a separation of the target subcellular component from other components of the sample:
collecting at least one fraction of the separated sample comprising the target subcellular component without further centrifugation and;
optionally imaging the target subcellular component in the sample prior to, during, and/or after the separation;
wherein the particles are from about 10 nanometers to about 15 microns in size; and
   wherein:
a) the particles form a complex with one or more of the contaminating species, or form a structure in the sample medium that interacts with one or more of the contaminating species, in a manner that inhibits the movement of the one or more contaminating species, in a chosen direction relative to the movement of the target subcellular component in the same direction; or
b) the particles form a complex with the one or more of the contaminating species, or form a structure in the sample medium that interacts with one or more of the contaminating species, in a manner to increase the movement of the one or more contaminating species, in a chosen direction relative to the movement of the target subcellular component in the same direction.

Provided herein is a method (“Method 3”) for separation of live cells and/or cell nuclei from a mixture comprising said live cells and/or cell nuclei, dead cells and cellular debris, comprising:
A) providing a fluidic sample processing device comprising,
   (i) a processing channel,
   (ii) an inlet channel,
   (iii) an inlet connection region connecting the inlet channel to the processing channel,
   (iv) a plurality of magnetic components aligned along the X-axis of the processing channel on the upper side and lower side of the processing channel,
   (v) a plurality of outlet channels,
   (vi) an outlet connection region connecting the processing channel to the outlet channels,
   (vii) a first outlet channel in fluidic communication with an upper region of the processing channel at an outlet connection region,
   (viii) a second outlet channel in fluidic communication with a lower region of the processing channel at an outlet connection region, and
   (ix) a first flow modulator associated with the first outlet channel and a second flow modulator associated with the second outlet channel; and
B) flowing the mixture through the fluidic sample processing device to provide a first recovered sample enriched in said cell nuclei and/or live cells; and a second recovered sample depleted in said cell nuclei and/or live cells;
wherein the mixture comprises:
   i) a paramagnetic compound or ferrofluid; and
   ii) isolation particles (or beads);
wherein the particles are from about 10 nanometers to about 15 microns in size; and
   wherein:
a) the particles form a complex with the cellular debris, or form a structure in the sample medium that interacts with the cellular debris, in a manner that inhibits the movement of at least a portion of the cellular debris in a chosen direction relative to the movement of the cell nuclei; or
b) the particles form a complex with the cellular debris, or form a structure in the sample medium that interacts with the cellular debris, in a manner to increase the movement of at least a portion of the cellular debris in a chosen direction relative to the movement of the cell nuclei.

Provided herein is a method (“Method 4”) for separation of live cells and/or cell nuclei from a mixture comprising said live cells and/or cell nuclei, dead cells and nuclear debris, comprising:
providing a flowcell cartridge comprising a processing channel, and a plurality of outlet channels wherein the outlet channels of the flowcell cartridge have a volume greater than the processing channel;
flowing a sample solution comprising live cells and dead cells and a paramagnetic compound into the processing channel;
placing the flowcell cartridge in a magnetic field substantially aligned parallel to the processing channel;
maintaining the processing channel and the sample contained therein entirely within the magnetic field in a stopped flow condition for a period of time sufficient to separate live cells and dead cells by a vertical distance within the processing channel; and
simultaneously withdrawing a sample fraction enriched with live cells and/or cell nuclei and a sample fraction enriched with dead cells and nuclear debris into the outlet channels;
wherein the sample solution comprises isolation particles that are from about 10 nanometers to about 15 microns in size; and wherein:
a) the isolation particles form a complex with the cellular debris, or form a structure in the sample medium that interacts with the cellular debris, in a manner that inhibits the movement of at least a portion of the cellular debris in a chosen direction relative to the movement of the cell nuclei; or
b) the isolation particles form a complex with the cellular debris, or form a structure in the sample medium that interacts with the cellular debris, in a manner to increase the movement of at least a portion of the cellular debris in a chosen direction relative to the movement of the cell nuclei.
   Provided herein is a kit (“Kit 1”) for isolating cell nuclei comprising one or more components selected from buffers, solvents, proteins, particles, enzymes, stabilizers and the like.

Provided herein are any of the foregoing Methods 2, 3 or 4 performed on a fluidic concentrator device that includes an inlet channel, a processing channel, and at least two output channels, and a pump for movement of a particle containing sample through the concentrator device. The concentrator device may have separate diversion channels that may be controlled by a valve or a functionally similar diversion technique to collect all of, or fractions of, a particle concentrated stream or a particle depleted stream. The concentrator device may be operable under automated control and further comprise one or more sensors inside, or adjacent to, portions of the processing channel or inlet channel to detect presence or absence or quantity of particles or other physical or chemical properties of the particles or sample flow stream. The output of the detectors may be operably-linked to concentrator controls to optimize concentration and fractionation conditions. Particle concentration/depletion may be physically accomplished by the device through gravitational sedimentation, magnetic levitation/repulsion, and a combination thereof. The interface of the inlet channel to the processing channel is preferably geometrically configured to reduce or eliminate turbulent flow in the processing channel. The interface between the processing channel and the output channels is preferably geometrically configured to facilitate collection of layered streams, a particle enriched stream and a particle depleted stream into their respective outlet channels.

Also provided herein any of the foregoing Methods 2, 3 or 4, performed on a fluidic concentrating device with a magnetic component positioned substantially linear along the processing channel (X-axis) to provide magnetic repulsion or attraction of the particles in the processing channel based upon their paramagnetic properties. The magnetic component may function to induce or augment sedimentation of the particles within the processing channel. An additional application of the fluidic concentrating device comprising a magnet component along the processing channel is the ability of such device to be operable in low- or micro-gravity environments. Alternatively, in some cases, the magnetic component may selectively inhibit sedimentation of certain particles in a sample fluid when the sample fluid is a heterogeneous particle mixture. Providing a magnetic field within the inlet channel may, in some embodiments, impart a preconcentrating effect by providing a surmountable inhibition of particle flow from the inlet channel into the processing channel. The inlet channel magnetic field can be induced extending a magnet that is substantially linear along the processing channel into the inlet channel. Alternatively, a magnetic component may be independent of the processing channel and placed in magnetic communication with the inlet channel. In one embodiment, this is a bar magnet, in another embodiment it is an annular or toroidal magnet surrounding all or a portion of the inlet channel. The inlet channel magnets may be permanent magnets or electromagnets under control of a magnetic controller. In further embodiments exemplified below, the processing channel may have a plurality of magnetic components placed substantially linear to the processing channel. In one such embodiment, magnets providing dissimilar magnetic field strength are positioned opposite each other (e.g. top and bottom) in a substantially linear fashion parallel to the processing channel. When combined with a preconcentration step or device configuration, this embodiment can act selectively to concentrate a predetermined particle component of a sample contain a heterogeneous particle composition. The accumulation of the particles outside the processing channel can be a passive process that is dependent on sample flowrate and the mobility of the particles in the sample liquid medium within the inlet channel. The accumulation of the particles outside the processing channel can be an active process utilizing a magnetic field within the inlet channel to impede particle within the field. The impediment of particle movement from the inlet channel into the processing channel can be overcome by manipulation of flowrate or flow pattern. For example, increasing particle mobility in the inlet channel through increase in flowrate or the introduction of one or more pulses of increased channel pressure. When particle inhibition is accomplished by electromagnetic field induction in the inlet channel, particle mobility from the inlet channel into the processing channel can be increased by reduction of the inlet channel magnetic field, modification of inlet channel flow rate or pressure, or a combination of magnetic field and sample flow properties.

Further provided herein any of the foregoing Methods 2, 3 or 4, performed on a particle-concentrating device comprising a fluidic processing channel structure, at least one magnetic component, and at least two output ports, wherein the fluidic processing channel includes a substantially linear portion having a leading end that is in fluidic communication with the input port and a tail end that is in fluidic communication with the output ports. The at least two output ports are substantially configured in parallel. In accordance with this embodiment, each of the output ports comprises at least one collection pathway, wherein the collection pathway leads to a collection chamber containing a determined quantity of a material required for a subsequent processing step. The fluidic channel structure is typically a micro-capillary channel, wherein particles are allowed to flow through freely or at a desired rate. The device may further comprise one or more pumps configured to drive fluid from an input port through the fluidic channel structure. In some embodiments, the device further comprises one or more valves for controlling the particle pathway and/or flow rate.

Embodiments of the methods of the present invention are further described in the numbered embodiments below. The numbered embodiments are non-limiting of the invention and may incorporate further elements and alternatives described herein.

### Embodiments of Method 1

Provided herein in a first embodiment is a method (“Method 1”) for extracting cellular nuclei from intact cells comprising; providing intact cells; and lysing the cells in a nuclei isolating buffer; wherein the nuclei isolating buffer comprises wheat germ agglutinin (WGA).

In a second embodiment (2), the WGA is present in an amount of from about 0.01 mg/mL to about 2 mg/mL; e.g., from about 0.01 mg/mL to about 1 mg/mL; e.g., from about 0.01 mg/mL to about 0.5 mg/mL; e.g., 0.05 mg/mL to about 0.15 mg/mL; e.g., about 0.1 mg/mL. In a third embodiment (3), the nuclei isolating buffer comprises said WGA and one or more of: buffers, solvents, proteins, particles, enzymes, stabilizers and solvents; and water.

In a fourth embodiment (4), the method further comprises collecting the nuclei, e.g. by centrifugation; and optionally storing the nuclei; for example by suspending the nuclei in a storage buffer; for example wherein the storage buffer comprises sucrose. In a fifth embodiment (5), the integrity of isolated cell nuclei is at least about 15% greater than the integrity of cell nuclei isolated by a similar method lacking WGA. In a sixth embodiment (6), the method further comprises suspending the collected nuclei in a levitation buffer containing a levitation agent. In a seventh embodiment (7), the levitation agent comprises 100 mM Gadolinium, and the levitation buffer comprises 1×PBS, 1% BSA, and a RNAse inhibitor, e.g. RNAseOUT™. In an eighth embodiment (8), the method further comprises performing any of the Methods 2 et seq., described herein utilizing the isolated cell nuclei.

In a further embodiment, the present disclosure provides a Method (“Method 1a”) of reducing contamination isolated cell nuclei from ambient RNA, comprising isolating the cell nuclei in the presence of wheat germ agglutinin (WGA). In a second embodiment (2), the WGA is present in an amount of from about 0.01 mg/mL to about 2 mg/mL; e.g., from about 0.01 mg/mL to about 1 mg/mL; e.g., from about 0.01 mg/mL to about 0.5 mg/mL; e.g., 0.05 mg/mL to about 0.15 mg/mL; e.g., about 0.1 mg/mL.

### Embodiments of Method 2

Provided herein is a method (“Method 2”) of isolation of a target subcellular component, e.g. cellular nuclei, from a sample comprising the target subcellular component and one or more contaminating species, comprising:
loading a sample comprising the target subcellular component, the contaminating species, and a sample medium comprising:
   i) a paramagnetic compound or ferrofluid; and
   ii) isolation particles or beads;
      into a separation well or channel along which the sample is optionally caused to flow;
subjecting the sample to a magnetic force with at least one magnet to affect a separation of the target subcellular component from other components of the sample;
collecting at least one fraction of the separated sample comprising the target subcellular component without further centrifugation and;
optionally imaging the target subcellular component in the sample prior to, during, and/or after the separation;
wherein the particles or beads are from about 10 nanometers to about 15 microns in size; and wherein:
a) the isolation particles or beads form a complex with one or more of the contaminating species, or form a structure in the sample medium that interacts with one or more of the contaminating species, in a manner that inhibits the movement of the one or more contaminating species in a chosen direction relative to the movement of the target subcellular component in the same direction; or
b) the isolation particles or beads form a complex with the one or more of the contaminating species, or form a structure in the sample medium that interacts with one or more of the contaminating species, in a manner to increase the movement of the one or more contaminating species in a chosen direction relative to the movement of the target subcellular component in the same direction.

In a second embodiment (2) of Method 2, the isolation particles or beads form a complex with one or more of the contaminating species, or form a structure in the sample medium that interacts with one or more of the contaminating species, in a manner that inhibits the movement of one or more of the contaminating species in the direction of ambient gravitational force relative to the movement of the target subcellular component in the same direction.

A third embodiment (3) of Method 2, is an embodiment according to any of the previous embodiments of Method 2, wherein the target subcellular component is cell nuclei. A fourth embodiment (4) of Method 2 is an embodiment according to any of the previous embodiments of Method 2, wherein the contaminating species comprises one or more of intact cells, dead cells, nuclear debris and cell fragments. A fifth embodiment (5) of Method 2 is an embodiment according to any of the previous embodiments of Method 2, wherein the isolation particles or beads are from about 10 nanometers to about 10 microns in size; e.g. about 1 to about 8 microns in size; e.g. about 1 to about 5 microns in size; e.g. about 1, about 2, about 3, about 4 or about 5 microns in size; from about 10 nanometers to about 1 micron in size; e.g. about 10 to about 800 nanometers in size; e.g. about 10 to about 500 nanometers in size; e.g. about 100, about 200, about 250, about 300, about 350, about 400, about 450, or about 500 nanometers in size. A sixth embodiment (6) of Method 2 is an embodiment according to any of the previous embodiments of Method 2, wherein the isolation particles are selected from agarose beads, e.g. Sepharose™ beads, dextran beads e.g. Sephadex™, polyacrylamide beads, dextrose beads, polystyrene beads, beads made from polymeric resins. e.g. polyvinylethylcarbitol, polyvinylpyrrolidone, cellulose, silica-based materials, and/or from mixtures such as dextran-polyacrylamide, e.g. Sephacryl™ beads; each of which can optionally be coated, for example with a protein.

A seventh embodiment (7) of Method 2 is an embodiment according to any of the previous embodiments of Method 2, wherein the isolation particles are optionally coated polystyrene particles; e.g., polystyrene particles coated with a protein. An eighth embodiment (8) of Method 2 is an embodiment according to any of the previous embodiments of Method 2, wherein the isolation particles are polystyrene particles coated with streptavidin. A ninth embodiment (9) of Method 2 is an embodiment according to any of the previous embodiments of Method 2, wherein the isolation particles have a size of about 3 microns; or from about 10 nanometers to about 1 micron in size; e.g. about 10 to about 800 nanometers in size; e.g. about 10 to about 500 nanometers in size; e.g. about 100, about 200, about 250, about 300, about 350, about 400, about 450, or about 500 nanometers in size. A tenth embodiment (10) of Method 2 is an embodiment according to any of the previous embodiments of Method 2, wherein the sample comprises from about 50 to about 10,000,000 cell nuclei.

An eleventh embodiment (11) of Method 2 is an embodiment according to any of the previous embodiments of Method 2, wherein the concentration of cell nuclei in a fraction is increased by at least 1.1:1 from the original sample. A twelfth embodiment (12) of Method 2 is an embodiment according to any of the previous embodiments of Method 2, wherein the concentration of non-nuclei particles in the original sample is decreased by at least about 1% in the fraction. A thirteenth embodiment (13) of Method 2 is an embodiment according to any of the previous embodiments of Method 2, wherein the integrity of isolated cell nuclei in a fraction from a sample is at least about 30% greater than the integrity of cell nuclei isolated in a fraction from a sample by a method comprising centrifugation.

A fourteenth embodiment (14) of Method 2 is an embodiment according to any of the previous embodiments of Method 2, wherein the cell nuclei are isolated from human cells, non-human animal cells, or plant cells. A fifteenth embodiment (15) of Method 2 is an embodiment according to any of the previous embodiments of Method 2, wherein the nuclei are isolated from healthy cells, diseased cells, infected cells, transfected cells, or genetically modified cells.

### Embodiments of Method 3

Provided herein is a method (“Method 3”) for separation of live cells and/or cell nuclei from a mixture comprising said live cells and/or cell nuclei, dead cells and cellular debris, comprising:
A) providing a fluidic sample processing device comprising,
   (i) a processing channel,
   (ii) an inlet channel,
   (iii) an inlet connection region connecting the inlet channel to the processing channel,
   (iv) a plurality of magnetic components aligned along the X-axis of the processing channel on the upper side and lower side of the processing channel,
   (v) a plurality of outlet channels,
   (vi) an outlet connection region connecting the processing channel to the outlet channels,
   (vii) a first outlet channel in fluidic communication with an upper region of the processing channel at an outlet connection region,
   (viii) a second outlet channel in fluidic communication with a lower region of the processing channel at an outlet connection region, and
   (ix) a first flow modulator associated with the first outlet channel and a second flow modulator associated with the second outlet channel; and
B) flowing the mixture through the fluidic sample processing device to provide a first recovered sample enriched in said cell nuclei and/or live cells; and a second recovered sample depleted in said cell nuclei and/or live cells;
wherein the mixture comprises:
   i) a paramagnetic compound or ferrofluid; and
   ii) isolation particles (or beads);
wherein the isolation particles (or beads) are from about 10 nanometers to about 15 microns in size; and wherein:
a) the isolation particles form a complex with the cellular debris, or form a structure in the sample medium that interacts with the cellular debris, in a manner that inhibits the movement of at least a portion of the cellular debris in a chosen direction relative to the movement of the cell nuclei: or
b) the isolation particles form a complex with the cellular debris, or form a structure in the sample medium that interacts with the cellular debris, in a manner to increase the movement of at least a portion of the cellular debris in a chosen direction relative to the movement of the cell nuclei.

A second embodiment (2) of Method 3 is an embodiment wherein the isolation particles form a complex with the cellular debris, or form a structure in the sample medium that interacts with the cellular debris, in a manner that inhibits the movement of at least a portion of the cellular debris in the direction of ambient gravitational force relative to the movement of the cell nuclei. A third embodiment (3) of Method 3 is an embodiment according to any of the previous embodiments of Method 3, wherein the first recovered sample is enriched in cell nuclei. A fourth embodiment (4) of Method 3 is an embodiment according to any of the previous embodiments of Method 3, wherein the first recovered sample is enriched in live cells. A fifth embodiment (5) of Method 3 is an embodiment according to any of the previous embodiments of Method 3, wherein the isolation particles are from about 1 to about 10 microns in size; e.g. about 1 to about 8 microns in size; e.g. about 1 to about 5 microns in size; e.g. about 1, about 2, about 3, about 4 or about 5 microns in size; or from about 10 nanometers to about 1 micron in size; e.g. about 10 to about 800 nanometers in size; e.g. about 10 to about 500 nanometers in size; e.g. about 100, about 200, about 250, about 300, about 350, about 400, about 450, or about 500 nanometers in size.

A sixth embodiment (6) of Method 3 is an embodiment according to any of the previous embodiments of Method 3, wherein the isolation particles are selected from agarose beads, e.g. Sepharose™ beads, dextran beads e.g. Sephadex™, polyacrylamide beads, dextrose beads, polystyrene beads, beads made from polymeric resins. e.g. polyvinylethylcarbitol, polyvinylpyrrolidone, cellulose, silica-based materials, and/or from mixtures such as dextran-polyacrylamide, e.g. Sephacryl™ beads; each of which can optionally be coated, for example with a protein. A seventh embodiment (7) of Method 3 is an embodiment according to any of the previous embodiments of Method 3, wherein the isolation particles are optionally coated polystyrene particles; e.g., polystyrene particles coated with a protein. An eighth embodiment of Method 3 is an embodiment according to any of the previous embodiments of Method 3, wherein the isolation particles are polystyrene particles coated with streptavidin.

A ninth embodiment (9) of Method 3 is an embodiment according to any of the previous embodiments of Method 3, wherein the isolation particles have a size of about 3 microns; or from about 10 nanometers to about 1 micron in size; e.g. about 10 to about 800 nanometers in size; e.g. about 10 to about 500 nanometers in size; e.g. about 100, about 200, about 250, about 300, about 350, about 400, about 450, or about 500 nanometers in size. A tenth embodiment (10) of Method 3 is an embodiment according to any of the previous embodiments of Method 3, wherein the sample comprises from about 50 to about 10,000,000 cell nuclei. An eleventh embodiment (11) of Method 3, is an embodiment according to any of the previous embodiments of Method 3, wherein the concentration of cell nuclei in a fraction is increased by at least 1.1:1 from the original sample. A twelfth embodiment (12) of Method 3 is an embodiment according to any of the previous embodiments of Method 3, wherein the concentration of non-nuclei particles in the original sample is decreased by at least about 1% in the fraction. A thirteenth embodiment (13) of Method 30 is an embodiment according to any of the previous embodiments of Method 3, wherein the integrity of isolated cell nuclei in a fraction from a sample is at least about 30% greater than the integrity of cell nuclei isolated in a fraction from a sample by a method comprising centrifugation.

A fourteenth embodiment (14) of Method 3 is an embodiment according to any of the previous embodiments of Method 3, wherein the cell nuclei are isolated from human cells, non-human animal cells, or plant cells. A fifteenth embodiment (15) of Method 3 is an embodiment according to any of the previous embodiments of Method 3, wherein the nuclei are isolated from healthy cells, diseased cells, infected cells, transfected cells, or genetically modified cells. A sixteenth embodiment (16) of Method 3 is an embodiment according to any of the previous embodiments of Method 3, wherein the yield of live cells in the first recovered sample is at least about 50%, at least about 60%, at least about 70%, or at least about 75% of the total live cell composition of the mixture; and/or the yield of nuclei in the first recovered sample is at least about 50%, at least about 60%, at least about 70%, or at least about 75% of the total nuclei from the live cell composition of the mixture.

A seventeenth embodiment (17) of Method 3 is an embodiment according to any of the previous embodiments of Method 3, wherein the outlet connection region further comprises a flow stream splitter portion. Embodiment eighteen (18) is an embodiment according to embodiment seventeen (17) of Method 3 wherein the flow stream splitter portion protrudes into the processing channel and is constructed and arranged to separate a fluidic stream into separate streams in the outlet channels.

A nineteenth embodiment (19) of Method 3 is an embodiment according to any of the previous embodiments of Method 3, wherein the fluidic sample processing device further comprises a first flowrate sensor associated with the first outlet channel and a second flowrate sensor associated with the second outlet channel. Embodiment twenty (20) of Method 3 is an embodiment according to embodiment 19, wherein a flowrate sensor is operatively linked to a flow modulator.

Embodiment twenty-one (21) is an embodiment according to any of the previous embodiments of Method 3, wherein the fluidic sample processing device further comprises an optical sensor and an illumination source configured opposite or angularly adjacent to the optical sensor; optionally wherein the illumination source emits ultraviolet light.

Embodiment twenty-two (22) is an embodiment according to any of the previous embodiments of Method 3, wherein the fluidic sample processing device further comprises a sensor wherein the sensor is a photodetector, a multipixel imaging detector, a magnetic field detector, an electrochemical detector, an optical phase detector, a scatter detector, a Hall sensor, a magnetoresistive sensor, a bolometric sensor, a surface acoustic wave sensor, a biosensor, a capacitive sensor, a conductive sensor, a thermal sensor, a flowrate sensor, an ultrasonic sensor, a gravimetric sensor, a magnetic field sensor or combinations thereof; and a controller operatively linked to plurality of flow modulators.

Embodiment twenty-three (23) is an embodiment according to any of the previous embodiments of Method 3, wherein the fluidic sample processing device comprises a flowcell cartridge comprising a planar substrate, said planar substrate comprising:
(i) an upper surface and a lower surface;
(ii) a first longitudinal side forming an imaging surface;
(iii) a second longitudinal side forming an illumination surface;

and
(iv) a first and second transverse side;
(v) an inlet well on an upper surface;
(vi) an inlet channel;
(vii) a sample processing channel in fluidic communication with the inlet channel and positioned substantially parallel to a longitudinal side;
(viii) a sample splitter within the processing channel;
(ix) a plurality of outlet channels in fluidic communication with the processing channel; and
(x) a plurality of collection wells in fluidic communication with each of the plurality of outlet channels;

wherein the substrate optionally comprises an optically transparent material and wherein the processing channel is offset within the plane of the of the substrate to be spatially biased to the imaging surface,

optionally wherein the substrate is comprised of nonferrous metal, ceramic, glass, polymer, or plastic; and

optionally wherein if the substrate comprises one or more layers, the substrate and planar layer may be comprised of the same or different material.

Embodiment twenty-four (24) is an embodiment according to any of the previous embodiments of Method 3, wherein:
the fluidic sample processing device comprises a flowcell cartridge comprising a planar substrate, said planar substrate comprising:
   (i) an inlet well on an upper surface;
   (ii) an inlet channel;
   (iii) a sample processing channel;
   (iv) a sample splitter within the processing channel;
   (v) a plurality of outlet channels in fluidic communication with the processing channel; and
   (vi) a plurality of collection wells in fluidic communication with each of the plurality of outlet channels;
   wherein the substrate comprises an optically transparent material and wherein the combined volume each of the plurality of outlet channels is greater than the volume of the processing channel;
   optionally wherein the substrate is comprised of nonferrous metal, ceramic, glass, polymer, or plastic; and
   optionally wherein if the substrate comprises one or more layers, the substrate and planar layer may be comprised of the same or different material.

Embodiment twenty-five (25) is an embodiment according to any of the previous embodiments of Method 3, wherein the outlet channels of the flow cell cartridge follow compacted paths, for example wherein the outlet channels are serpentine channels. Embodiment twenty-six (26) is an embodiment according to embodiments 23-25 of Method 3, wherein the outlet channels of the flowcell cartridge are formed as recesses within the planar substrate and a first outlet channel comprises a recess on a surface of the planar substrate and a second outlet channel comprises a recess on an opposite side of the planar substrate; optionally wherein the channels are formed by etching, machining, 3D printing, or molding the planar substrate; and/or the flowcell further comprised one or more additional planar layers positioned over the recesses in the planar substrate to form enclosed channels; optionally wherein the one or more planar layers are attached to the planar substrate by compression, adhesive bonding, preferably a biocompatible adhesive, more preferably a silicone or silicone-based adhesive, solvent bonding, ultrasonic welding, thermal bonding, welding, or 3D printing.

Embodiment twenty-seven (27) is an embodiment according to embodiments 23-26 of Method 3, wherein the planar substrate comprises a polymer material, for example cyclic olefin polymer or cyclic olefin copolymer; and further comprising: a collection well formed on the planar substrate and in fluidic communication with a terminal portion of an outlet channel; and/or an internal channel inlet at a first well height and an internal outlet at a second well height wherein the inlet is in fluidic communication with an outlet channel of the flowcell cartridge and wherein the second well height is higher than the first well height.

### Embodiments of Method 4

Provided herein is a method (“Method 4”) for separation of live cells and/or cell nuclei from a mixture comprising said live cells and/or cell nuclei, dead cells and nuclear debris, comprising:
providing a flowcell cartridge comprising a processing channel, and a plurality of outlet channels wherein the outlet channels of the flowcell cartridge have a volume greater than the processing channel;
flowing a sample solution comprising live cells and/or cell nuclei, dead cells and nuclear debris, and a paramagnetic compound into the processing channel;
placing the flowcell cartridge in a magnetic field substantially aligned parallel to the processing channel;
maintaining the processing channel and the sample contained therein entirely within the magnetic field in a stopped flow condition for a period of time sufficient to separate live cells and dead cells by a vertical distance within the processing channel; and
simultaneously withdrawing a sample fraction enriched with live cells and/or cell nuclei and a sample fraction enriched with dead cells and/or intact cells and nuclear debris into the outlet channels;
wherein the sample solution and/or flow stream further comprises isolation particles that are from about 10 nanometers to about 15 microns in size; and wherein:
a) the isolation particles form a complex with the cellular debris, or form a structure in the sample medium that interacts with the cellular debris, in a manner that inhibits the movement of at least a portion of the cellular debris in a chosen direction relative to the movement of the cell nuclei; or
b) the isolation particles form a complex with the cellular debris, or form a structure in the sample medium that interacts with the cellular debris, in a manner to increase the movement of at least a portion of the cellular debris in a chosen direction relative to the movement of the cell nuclei.

A second embodiment (2) of Method 4 is an embodiment wherein the isolation particles form a complex with the cellular debris, or form a structure in the sample medium that interacts with the cellular debris, in a manner that inhibits the movement of at least a portion of the cellular debris in the direction of ambient gravitational force relative to the movement of the cell nuclei. A third embodiment (3) of Method 4 is an embodiment according to any preceding embodiment of Method 4, further comprising providing a flowcell cartridge that is substantially free of any liquid or paramagnetic compound prior to introduction of the sample solution. A fourth embodiment (4) of Method 4 is an embodiment according to any preceding embodiment of Method 4, wherein the isolation particles are from about 10 nanometers to about 10 microns in size; e.g. about 1 to about 8 microns in size; e.g. about 1 to about 5 microns in size; e.g. about 1, about 2, about 3, about 4 or about 5 microns in size; from about 10 nanometers to about 1 micron in size; e.g. about 10 to about 800 nanometers in size; e.g. about 10 to about 500 nanometers in size; e.g. about 100, about 200, about 250, about 300, about 350, about 400, about 450, or about 500 nanometers in size.

A fifth embodiment (5) of Method 4 is an embodiment according to any preceding embodiment of Method 4, wherein the isolation particles are selected from agarose beads, e.g. Sepharose™ beads, dextran beads e.g. Sephadex™, polyacrylamide beads, dextrose beads, polystyrene beads, beads made from polymeric resins. e.g. polyvinylethylcarbitol, polyvinylpyrrolidone, cellulose, silica-based materials, and/or from mixtures such as dextran-polyacrylamide, e.g. Sephacryl™ beads; each of which can optionally be coated, for example with a protein.

A sixth embodiment (6) of Method 4 is an embodiment according to any preceding embodiment of Method 4, wherein the isolation particles are optionally coated polystyrene particles; e.g., polystyrene particles coated with a protein. A seventh embodiment (7) of Method 4 is an embodiment according to any preceding embodiment of Method 4, wherein the isolation particles are polystyrene particles coated with streptavidin. An eighth embodiment (8) of Method 4 is an embodiment according to any preceding embodiment of Method 4, wherein the isolation particles have a size of about 3 microns; or from about 10 nanometers to about 1 micron in size; e.g. about 10 to about 800 nanometers in size; e.g. about 10 to about 500 nanometers in size; e.g. about 100, about 200, about 250, about 300, about 350, about 400, about 450, or about 500 nanometers in size. A ninth embodiment (9) of Method 4 is an embodiment according to any preceding embodiment of Method 4, wherein the sample comprises from about 50 to about 10,000,000 cell nuclei. A tenth embodiment (10) of Method 4 is an embodiment according to any preceding embodiment of Method 4, wherein the concentration of cell nuclei in a fraction is increased by at least 1.1:1 from the original sample.

An eleventh embodiment (11) of Method 4 is an embodiment according to any preceding embodiment of Method 4, wherein the concentration of non-nuclei particles in the original sample is decreased by at least about 1% in the fraction. A twelfth embodiment (12) of Method 4 is an embodiment according to any preceding embodiment of Method 4, wherein the integrity of isolated cell nuclei in a fraction from a sample is at least about 30% greater than the integrity of cell nuclei isolated in a fraction from a sample by a method comprising centrifugation. A thirteenth embodiment (13) of Method 4 is an embodiment according to any preceding embodiment of Method 4, wherein the cell nuclei are isolated from human cells, non-human animal cells, or plant cells. A fourteenth embodiment (14) of Method 4 is an embodiment according to any preceding embodiment of Method 4, wherein the nuclei are isolated from healthy cells, diseased cells, infected cells, transfected cells, or genetically modified cells.

A fifteenth embodiment (15) of Method 4 is an embodiment according to any preceding embodiment of Method 4, further comprising providing a flowcell cartridge wherein the outlet channels have a cross sectional area less than the cross sectional area of the processing channel and are arranged to follow compacted paths, one exemplary configuration being a serpentine channel.

A sixteenth embodiment (16) of Method 4 is an embodiment according to any preceding embodiment of Method 4, further comprising providing a magnetic field in close proximity to the top vertical surface of the processing channel and in close proximity to the bottom vertical surface of the processing channel, each magnetic field have similar strength and surface field strength of between about 0.8 Tesla and about 2.0 Tesla and optionally between about 0.9 Tesla and about 1.4 Tesla.

A seventeenth embodiment (17) of Method 4 is an embodiment according to any preceding embodiment of Method 4, further comprising providing a paramagnetic compound in the sample solution at a concentration of from about 50 mM to about 200 mM, optionally from about 65 mM to about 175 mM, and further optionally from about 70 mM to about 150 mM.

A eighteenth embodiment (19) of Method 4 is an embodiment according to any preceding embodiment of Method 4, further comprising the step of withdrawing the sample fractions into the outlet channels at a flow rate of from about 75 uL per minute to about 150 uL per minute, and optionally at about 75 uL per minute, about 90 uL per minute, about 100 μL per minute, about 110 uL per minute, about 120 uL per minute, or about 150 uL per minute.

A nineteenth embodiment (19) of Method 4 is an embodiment according to any preceding embodiment of Method 4, wherein the enriched recovered sample fraction comprises at least about 60%, at least about 70%, at least about 80% or at least about 90% live cells.

A twentieth embodiment (20) of Method 4 is an embodiment according to any preceding embodiment of Method 4, wherein the yield of live cells in the enriched recovered sample fraction is at least about 50%, at least about 60%, at least about 70%, or at least about 75% of the total live cell composition of the sample.

A twenty-first embodiment (21) of Method 4 is an embodiment according to any preceding embodiment of Method 4, wherein the yield of nuclei in the enriched recovered sample fraction is at least about 50%, at least about 60%, at least about 70%, or at least about 75% of the total nuclei from the live cell composition of the sample.

### FURTHER METHOD EMBODIMENTS

Provided herein is a method (Method 5) for concentrating a sample of cells and/or cell nuclei, comprising, (i) providing a low volume fluidic device with a processing channel, an inlet channel, and a plurality of outlet channels, (ii) flowing a cells and/or cell nuclei containing sample through the inlet channel into the processing channel under conditions to produce a sample flow stream with at least a cells and/or cell nuclei enriched layer and a cells and/or cell nuclei depleted layer, (iii) flowing the cells and/or cell nuclei enriched layer through a first outlet channel to produce a cells and/or cell nuclei enriched flow stream, (iv) flowing the cells and/or cell nuclei depleted layer through a second outlet channel to produce a cells and/or cell nuclei depleted flow stream, and (v) collecting one or more of the flow streams from one or more of the outlet channels; wherein the sample and/or flow stream comprises isolation particles that are from about 10 nanometers to about 15 microns in size; and wherein:
a) the isolation particles form a complex with one or more non-nuclei components in the sample, or form a structure in the flow stream that interacts with one or more non-nuclei components in the sample, in a manner that inhibits the movement of at one or more non-nuclei components in the sample in a chosen direction relative to the movement of the cell nuclei; or
b) the isolation particles form a complex with one or more non-nuclei components in the sample, or form a structure in the sample medium that interacts with one or more non-nuclei components in the sample, in a manner to increase the movement of one or more non-nuclei components in the sample in a chosen direction relative to the movement of the cell nuclei.

A second embodiment (2) of Method 5 is the first embodiment further comprising subjecting the cells and/or cell nuclei containing sample to flow conditions sufficient to induce sedimentation of the sample cells and/or cell nuclei into the cells and/or cell nuclei enriched flow stream. A third embodiment (3) of Method 5 is the first embodiment (1) further comprising providing a magnetic field from the top of the processing channel and aligned with the X-axis of the processing channel and repelling cells and/or cell nuclei in the sample into cells and/or cell nuclei enriched flow stream. A fourth embodiment (4) of Method 5 is embodiment three or four further comprising (i) inducing a magnetic field within the inlet channel to impede the movement of cells and/or cell nuclei from the inlet channel into the processing channel to form a cells and/or cell nuclei concentrated portion of the sample flow stream in the inlet channel, (ii) moving the cells and/or cell nuclei concentrated portion of the sample flow stream into the processing channel, (iii) producing a cells and/or cell nuclei enriched flow stream, (iv) flowing the cells and/or cell nuclei enriched flow stream through an outlet channel, and (v) capturing the cells and/or cell nuclei enriched flow stream.

A fifth embodiment (5) of Method 5 is method of the embodiments of two or three (2-3) further comprising (i) inducing a magnetic field within the inlet channel to impede the movement of cells and/or cell nuclei from the inlet channel into the processing channel to form a cells and/or cell nuclei concentrated segment of the sample flow stream in the inlet channel, (ii) moving the unimpeded portion of the sample flow stream into the processing channel, (iii) producing a cells and/or cell nuclei depleted flow stream, (iv) flowing the cells and/or cell nuclei depleted flow stream through an outlet channel, and (v) capturing the cells and/or cell nuclei depleted flow stream.

A sixth embodiment (6) of Method 5 is an embodiment of one through four (1-4) further comprising measuring the cells and/or cell nuclei in the cells and/or cell nuclei enriched layer in the processing channel to determine a relative cells and/or cell nuclei concentration or position, and collecting a fraction of the cells and/or cell nuclei enriched layer based on a high relative cells and/or cell nuclei concentration or position of cells and/or cell nuclei in the processing channel. A seventh embodiment (7) of Method 5 is the embodiments of one through three (1-3) or embodiment five (5) further comprising measuring the cells and/or cell nuclei in the cells and/or cell nuclei depleted layer in the processing channel to determine a relative cells and/or cell nuclei concentration, and collecting a fraction of the cells and/or cell nuclei depleted layer based on a low relative cells and/or cell nuclei concentration.

An eight embodiment (8) of Method 5 is embodiment five (5) further comprising providing the magnetic field within the inlet channel that is continuous with a magnetic field produced from the top of the processing channel and aligned along the X-axis of the processing channel. A ninth embodiment (9) of Method 5 is embodiment five (5) further comprising producing a toroidal magnetic field surrounding the inlet channel. A tenth embodiment (10) of Method 5 is the method of embodiment nine (9) further comprising modulating the magnetic field within the inlet channel to facilitate movement of the cells and/or cell nuclei enriched segment within inlet channel into the processing channel.

An eleventh embodiment (11) of Method 5 is a method of embodiments one through ten (1-10) further comprising detecting a cell and/or cell nuclei property within the processing channel and modulating the sample flowrate to manipulate concentration of cells and/or cell nuclei within the cells and/or cell nuclei enriched flow stream. Embodiment twelve (12) of Method 5 is embodiment eleven (11) further comprising detecting a cells and/or cell nuclei property within the cells and/or cell nuclei enriched flow stream and modulating the sample flowrate to manipulate concentration of cells and/or cell nuclei within the cells and/or cell nuclei enriched flow stream. Embodiment thirteen (13) of Method 5 is a method of embodiments ten or twelve (10 or 12) further comprising detecting a cell and/or cell nuclei property within the processing channel and modulating the magnetic field within the inlet channel to manipulate concentration of cells and/or cell nuclei within the cells and/or cell nuclei enriched flow stream.

A fourteenth embodiment (14) of Method 5 is the method of embodiment thirteen (13) further comprising detecting a cell and/or cell nuclei property within the cells and/or cell nuclei enriched flow stream and modulating the magnetic field within the inlet channel to manipulate concentration of cells and/or cell nuclei within the cells and/or cell nuclei enriched flow stream.

A fifteenth embodiment (15) of Method 5 is a method of embodiments one through ten (1-10) further comprising detecting a cell and/or cell nuclei property within the inlet channel and modulating the sample flowrate to manipulate concentration of cells and/or cell nuclei within the cells and/or cell nuclei enriched flow stream. A sixteenth embodiment (16) of Method 5 is the method of embodiment ten (10) further comprising detecting a cell and/or cell nuclei property within the inlet channel and modulating the magnetic field within the inlet channel to manipulate concentration of cells and/or cell nuclei within the cells and/or cell nuclei enriched flow stream. A seventeenth embodiment (17) of Method 5 is a method according to embodiments eleven through sixteen (11-16) further comprising detecting relative cells and/or cell nuclei concentration or cells and/or cell nuclei density. Embodiment eighteen (18) of Method 5 is a method according to embodiments ten through seventeen (10-17) further comprising detecting a chemical property within the processing channel and modulating the sample flowrate to manipulate concentration of cells and/or cell nuclei within the cells and/or cell nuclei enriched flow stream. A nineteenth embodiment (19) of Method 5 is a method according to embodiments ten through seventeen (10-17) further comprising detecting a chemical property within the cells and/or cell nuclei enriched flow stream and modulating the sample flowrate to manipulate concentration of cells and/or cell nuclei within the cells and/or cell nuclei enriched flow stream.

A twentieth embodiment (20) of Method 5 is a method according to embodiments ten through seventeen (10-17) further comprising detecting a chemical property within the processing channel and modulating the magnetic field within the inlet channel to manipulate concentration of cells and/or cell nuclei within the cells and/or cell nuclei enriched flow stream. A twenty-first embodiment (21) of Method 5 is the method of embodiment twenty (20) further comprising detecting a chemical property within the cells and/or cell nuclei enriched flow stream and modulating the magnetic field within the inlet channel to manipulate concentration of cells and/or cell nuclei within the cells and/or cell nuclei enriched flow stream. A twenty-second embodiment (22) of Method 5 is a method of embodiments one through twenty-one (1-21) further comprising detecting a chemical property within the inlet channel and modulating the sample flowrate to manipulate concentration of cells and/or cell nuclei within the cells and/or cell nuclei enriched flow stream.

Embodiment twenty-three (23) of Method 5 is a method according to embodiments ten through twenty-two (10-22) further comprising detecting a chemical property within the inlet channel and modulating the magnetic field within the inlet channel to manipulate concentration of cells and/or cell nuclei within the cells and/or cell nuclei enriched flow stream.

Embodiment twenty-four (24) of Method 5 is a method of embodiments eighteen to twenty-three (18-23) wherein the property is an electrochemical, photonic, spectroscopic, or binding property. Embodiment twenty-five (25) of Method 5 is a method according to embodiments one through twenty-four (1-24) further comprising diverting the cells and/or cell nuclei depleted flow stream into a collection channel and capturing a fraction of the cells and/or cell nuclei depleted flow stream.

Embodiment twenty-six (26) of Method 5 is a method according to embodiments one through twenty-five (1-25) further comprising diverting the cells and/or cell nuclei enriched flow stream into a collection channel and capturing a fraction of the cells and/or cell nuclei enriched flow stream.

Embodiment twenty-seven (27) of Method 5 is a method according to embodiments one through twenty-six (1-26) further comprising diverting the cells and/or cell nuclei depleted flow stream and diverting the cells and/or cell nuclei enriched flow stream into a respective collection channels and capturing a fraction of each flow stream. Embodiment twenty-eight (28) of Method 5 is a method according to embodiment twenty-seven (27) further comprising capturing multiple discrete fractions of the flow stream. Embodiment twenty-nine (29) of Method 5 is a method according to embodiments twenty-seven or twenty-eight (27 or 28) further comprising capturing nonsimultaneous fractions from the cells and/or cell nuclei depleted flow stream and the cells and/or cell nuclei enriched flow stream.

Embodiment thirty (30) of Method 5 is a method according to embodiments one through twenty-nine (1-29) further comprising adding a paramagnetic compound to the sample prior to introduction to the inlet channel. Embodiment thirty-one (31) of Method 5 is a method according to embodiments one through thirty (1-30) further comprising performing a subsequent reaction on an isolated fraction. Embodiment thirty-two (32) of Method 5 is the method of embodiment thirty-one (31) wherein the subsequent reaction is a binding, a PCR, a sequencing sample preparation, enzymatic degradation, or enzymatic synthesis reaction. Embodiment thirty-three (33) of Method 5 is the method of embodiment thirty-one (31) wherein the collected sample is subjected to cell culture, florescence-activated cell sorting, or magnetic levitation cell sorting.

Embodiment thirty-four (34) of Method 5 is a method according to any of embodiments one through thirty-three (1-33) wherein the sample fluid is first flowed at an angle that is substantially not linearly aligned to the processing channel and then flowed at an angle that is substantially linear with the processing channel.

Embodiment thirty-five (35) of Method 5 is a method of fractionating a blood sample comprising (i) providing a whole blood sample or diluted blood sample, and (ii) subjecting the sample to a sample concentration method of embodiments one through thirty-four (1-34) and isolating plasma and/or blood cells from a whole or diluted blood sample. A thirty-sixth embodiment (36) of Method 5 is the method of embodiment thirty-five (35) wherein the blood sample is of a volume of from about 50 uL to about 10 mL. A thirty-seventh embodiment (37) of Method 5 is a method of embodiment thirty-six (36) wherein the plasma fraction contains less than about 1% of the blood cells in the blood sample. A thirty-eighth embodiment (38) of Method 5 is the method of embodiment thirty-seven (37) wherein the plasma fraction contains less than about 0.01% of the blood cells in the blood sample. A thirty-ninth embodiment (39) of Method 5 is the method of embodiment thirty-eight (38) wherein the plasma fraction is substantially free of the blood cells in the blood sample. A fortieth embodiment (40) of Method 5 is a method according to embodiments thirty-five through thirty-nine (35-39) wherein the blood sample is a peripheral blood sample, umbilical cord blood sample, fetal blood sample, or arterial blood sample. A forty-first embodiment (41) of Method 5 is a method according to embodiments thirty-five through forty (35-40) further comprising performing a diagnostic assay on an isolated fraction. A forty-second embodiment (42) of Method 5 is a method according to embodiment forty-one (41) wherein the assay is an enzyme immunoassay, chemiluminescent immunoassay, hemagglutination/particle agglutination assay, nucleic acid amplification technology assay, a drug assay, a forensic assay, or a genetic trait assay. Embodiment forty-three (43) of Method 5 is an embodiment according to any method of embodiments one through forty-one (1-41) where a reaction performed on the cells and/or cell nuclei or components of the cells and/or cell nuclei depleted layer within the inlet channel and/or the processing channel and, optionally concurrent with cells and/or cell nuclei isolation/concentration. Embodiment forty-four (44) of Method 5 is an embodiment according to method embodiment forty-three (43) wherein the reaction is a binding or staining reaction.

Embodiment forty-five (45) of Method 5 is an embodiment according to any one of embodiments one (1) to forty-four (44), wherein the particles are cell nuclei.

### Embodiments of Method 6

Provided herein is a method (“Method 6”) of isolation of a target subcellular component, e.g. cellular nuclei, from a sample comprising the target subcellular component and one or more contaminating species, comprising:
loading a sample comprising the target subcellular component, the contaminating species, and a sample medium comprising:
   i) a paramagnetic compound or ferrofluid; and
   ii) isolation particles or beads;
      into a separation well or channel along which the sample is optionally caused to flow;
subjecting the sample to a magnetic force with at least one magnet to affect a separation of the target subcellular component from other components of the sample;
collecting at least one fraction of the separated sample comprising the target subcellular component without further centrifugation and;
optionally imaging the target subcellular component in the sample prior to, during, and/or after the separation;
wherein the particles are from about 10 nanometers to about 1 micron in size; and
   wherein:
a) the isolation particles form a complex with one or more of the contaminating species, or form a structure in the sample medium that interacts with one or more of the contaminating species, in a manner that inhibits the movement of the one or more contaminating species in a chosen direction relative to the movement of the target subcellular component in the same direction; or
b) the isolation particles form a complex with the one or more of the contaminating species, or form a structure in the sample medium that interacts with one or more of the contaminating species, in a manner to increase the movement of the one or more contaminating species in a chosen direction relative to the movement of the target subcellular component in the same direction; or
c) the isolation particles form a complex with the one or more of the contaminating species, or form a structure in the sample medium that interacts with one or more of the contaminating species, in a manner to substantially deplete the contaminating species from the sample.

In a second embodiment (2) of Method 6, the isolation particles form a complex with one or more of the contaminating species, or form a structure in the sample medium that interacts with one or more of the contaminating species, in a manner that inhibits the movement of one or more of the contaminating species in the direction of ambient gravitational force relative to the movement of the target subcellular component in the same direction.

A third embodiment (3) of Method 6, is an embodiment according to any of the previous embodiments of Method 6, wherein the target subcellular component is cell nuclei.

A fourth embodiment (4) of Method 6 is an embodiment according to any of the previous embodiments of Method 6, wherein the contaminating species comprises one or more of intact cells, dead cells, nuclear debris and cell fragments.

A fifth embodiment (5) of Method 6 is an embodiment according to any of the previous embodiments of Method 6, wherein the isolation particles are from about 10 nanometers to about 1 micron in size; e.g. about 10 to about 800 nanometers in size; e.g. about 10 to about 500 nanometers in size: e.g. about 100, about 200, about 250, about 300, about 350, about 400, about 450, or about 500 nanometers in size.

A sixth embodiment (6) of Method 6 is an embodiment according to any of the previous embodiments of Method 6, wherein the isolation particles are selected from agarose beads, e.g. Sepharose™ beads, dextran beads e.g. Sephadex™, polyacrylamide beads, dextrose beads, polystyrene beads, beads made from polymeric resins. e.g. polyvinylethylcarbitol, polyvinylpyrrolidone, cellulose, silica-based materials, and/or from mixtures such as dextran-polyacrylamide, e.g. Sephacryl™ beads; each of which can optionally comprise a ferromagnetic material to become susceptible to magnetism e.g. ferromagnetic particles, and optionally be coated, for example with a protein.

A seventh embodiment (7) of Method 6 is an embodiment according to any of the previous embodiments of Method 6, wherein the isolation particles are optionally coated polystyrene particles; e.g., polystyrene particles coated with a protein.

An eighth embodiment (8) of Method 6 is an embodiment according to any of the previous embodiments of Method 6, wherein the isolation particles are polystyrene particles coated with streptavidin.

A ninth embodiment (9) of Method 6 is an embodiment according to any of the previous embodiments of Method 6, wherein the isolation particles have a size of from about 200 nanometers to about 400 nanometers.

A tenth embodiment (10) of Method 6 is an embodiment according to any of the previous embodiments of Method 6, wherein the sample comprises from about 50 to about 10,000,000 cell nuclei.

An eleventh embodiment (11) of Method 6 is an embodiment according to any of the previous embodiments of Method 6, wherein the concentration of cell nuclei in a fraction is increased by at least 1.1:1 from the original sample.

A twelfth embodiment (12) of Method 6 is an embodiment according to any of the previous embodiments of Method 6, wherein the concentration of non-nuclei particles in the original sample is decreased by at least about 19% in the fraction. A thirteenth embodiment (13) of Method 6 is an embodiment according to any of the previous embodiments of Method 6, wherein the integrity of isolated cell nuclei in a fraction from a sample is at least about 30% greater than the integrity of cell nuclei isolated in a fraction from a sample by a method comprising centrifugation.

A fourteenth embodiment (14) of Method 6 is an embodiment according to any of the previous embodiments of Method 6, wherein the cell nuclei are isolated from human cells, non-human animal cells, or plant cells.

A fifteenth embodiment (15) of Method 6 is an embodiment according to any of the previous embodiments of Method 6, wherein the nuclei are isolated from healthy cells, diseased cells, infected cells, transfected cells, or genetically modified cells.

A sixteenth embodiment (16) of Method 6 is an embodiment according to any of the previous embodiments of Method 6, wherein the sample volume in the separation well or channel is from about 50 uL to about 1 mL, about 75 uL to about 750 uL, about 100 uL to about 650 uL, about 150 uL to about 500 uL, about 200 uL to about 400 uL, or about 75 uL, about 100 uL, about 125 uL, about 150 uL, about 200 uL, about 250 uL, about 400 uL, about 500 uL, about 750 uL, or about 1 mL.

A seventeenth embodiment (17) of Method 6 is an embodiment according to any of the previous embodiments of Method 6, wherein the sample comprises about 10,000 to about 50,000 nuclei, from about 50,000 to about 100,000 nuclei, from about 100,000 to about 500,000 nuclei, from about 500,000 to about 800,000 nuclei, or greater than about 800,000 nuclei.

An eighteenth embodiment (18) of Method 6 is an embodiment according to any of the previous embodiments of Method 6, wherein the sample comprises about 1 μg to about 50 μg of isolation particles, from about 2 μg to about 25 μg of isolation particles, from about 2 μg to about 20 μg of isolation particles, or about 2 μg to about 15 μg of isolation particles.

A nineteenth embodiment of Method 6 is an embodiment according to any of the previous embodiments of Method 6, wherein the sample comprises WGA in a concentration of about 10 μg/mL to about 1 mg/mL, about 20 μg/mL to about 500 μg/mL, about 25 μg/mL to about 250 μg/mL, about 40 μg/mL to about 140 μg/mL. or about 25 μg/mL, about 50 μg/mL, about 60 μg/mL, about 75 μg/mL, or about 100 μg/mL.

A twentieth embodiment (20) of Method 6 is an embodiment according to any of the previous embodiments of Method 6, wherein the sample comprises a paramagnetic compound in solution at a concentration of from about 20 mM to about 400 mM, about 50 mM to about 300 mM, about 75 mM to about 200 mM, or about 75 mM, about 100 mM, about 125 mM, about 150 mM, or about 175 mM.

A twenty first embodiment (21) of Method 6 is an embodiment according to any of the previous embodiments of Method 6, wherein the isolating particles are ferromagnetic particles and form a complex with the contaminating particles, such that when the sample in the well or channel is placed within a magnetic force of the at least one magnet, the complex is attracted to one or more sides of the well or channel.

In some embodiments of the foregoing Methods, e.g., Method 6, the Method is performed in a fluidic concentrator device or system for conducting magnetic levitation separation of samples in a multi-well plate, as disclosed in International Application Ser. No. PCT/US2024/019795, filed Mar. 13, 2024, incorporated by reference herein in its entirety.

In some embodiments of each of the Methods described herein, the Method further comprises lysing cells in a nuclei isolating buffer; wherein the nuclei isolating buffer comprises wheat germ agglutinin (WGA). In some such embodiments, the WGA is present in an amount of from about 0.01 mg/mL to about 2 mg/mL; e.g., from about 0.01 mg/mL to about 1 mg/mL; e.g., from about 0.01 mg/mL to about 0.5 mg/mL; e.g., 0.05 mg/mL to about 0.15 mg/mL; e.g., about 0.1 mg/mL. In some further such embodiments, the nuclei isolating buffer comprises said WGA and one or more of: buffers, solvents, proteins, particles, enzymes, stabilizers and solvents; and water.

### Kit Embodiments

Provided herein is a kit (“Kit 1”) for isolating cell nuclei comprising a nuclei isolation buffer and one or more additional buffers, solvents, proteins, particles, enzymes, and/or stabilizers. A second embodiment (2) of Kit 1 is an embodiment wherein the isolation particles comprise beads, for example polystyrene beads, optionally coated with a protein, e.g. streptavidin, having a size of from about 1 micron to about 10 microns; e.g., about 1 to about 8 microns in size; e.g., about 1 to about 5 microns in size; e.g., about 1, about 2, about 3, about 4 or about 5 microns in size.

A third embodiment (3) of Kit 1 is an embodiment according to any preceding embodiment of Kit 1, wherein the isolation particles comprise beads, about 1 to about 8 microns in size; e.g., about 1 to about 5 microns in size; e.g., about 1, about 2, about 3, about 4 or about 5 microns in size; or from about 10 nanometers to about 1 micron in size: e.g. about 10 to about 800 nanometers in size; e.g. about 10 to about 500 nanometers in size; e.g. about 100, about 200, about 250, about 300, about 350, about 400, about 450, or about 500 nanometers in size. A fourth embodiment (4) of Kit 1 is an embodiment according to any preceding embodiment of Kit 1, wherein the isolation particles having a size of about 3 microns.

A fifth embodiment (5) of Kit 1 is an embodiment according to any preceding embodiment of Kit 1, wherein one or more of the components of the kit are provided as solids for reconstitution. A sixth embodiment (6) of Kit 1 is an embodiment according to any preceding embodiment of Kit 1, further comprising a levitation agent comprising gadolinium.

### Device Embodiments for Practicing the Methods of the Invention

Further embodiments of a device suitable for the practice of the methods disclosed herein are further described in the numbered embodiments below. The numbered embodiments are non-limiting of the invention and may incorporate further elements and alternatives described herein.

A first embodiment (1) is a magnetic fluidic sample processing device comprising, (i) a processing channel, (ii) an inlet channel, (iii) an inlet connection region connecting the inlet channel to the processing channel, (iv) a plurality of outlet channels, (v) an outlet connection region connecting the processing channel to the outlet channels, (vi) a first outlet channel in fluidic communication with an upper region of the processing channel at an outlet connection region, (vii) a second outlet channel in fluidic communication with a lower region of the processing channel at an outlet connection region, and (viii) a magnet aligned along the X-axis of the processing channel on either the upper side or the lower side of the processing channel.

A second embodiment (2) is a magnetic fluidic sample processing device comprising, (i) a processing channel, (ii) an inlet channel, (iii) an inlet connection region connecting the inlet channel to the processing channel. (iv) a plurality of outlet channels, (v) an outlet connection region connecting the processing channel to the outlet channels, (vi) a first outlet channel in fluidic communication with an upper region of the processing channel at an outlet connection region, (vii) a second outlet channel in fluidic communication with a lower region of the processing channel at an outlet connection region, and (viii) a plurality of magnetic components aligned along the X-axis of the processing channel on the upper side and lower side of the processing channel, wherein the processing device is constructed and arranged to provide preconcentration of particles prior to introduction into the processing channel.

A third embodiment (3) is a fluidic sample processing device comprising (i) a processing channel, (ii) an inlet channel, (iii) an inlet connection region connecting the inlet channel to the processing channel, (iv) a plurality of outlet channels, (v) an outlet connection region connecting the processing channel to the outlet channels, (vi) a first outlet channel in fluidic communication with an upper region of the processing channel at an outlet connection region, (vii) a second outlet channel in fluidic communication with a lower region of the processing channel at an outlet connection region, and (viii) an inlet channel flow controller.

A fourth embodiment (4) is the device of embodiment two wherein the magnetic components on the upper side of the processing channel and the lower side of the processing channel are constructed and arranged to provide a magnetic field of dissimilar strength within the processing channel.

A fifth embodiment (5) is a device according to embodiments one through four (1-4) wherein the inlet channel comprises a first cross-sectional area and the processing channel comprises a second cross-sectional area, the first cross-sectional area being less than the second cross-sectional area. A sixth embodiment (6) is a device according to embodiment five (5) wherein the channels are microfluidic or capillary.

A seventh embodiment (7) is a device according to embodiments one through six (1-6) wherein the inlet connection region is tapered at an angle of less than 90 degrees. In an eighth embodiment (8) a device is provided of embodiment seven (7) wherein the angle is equal to or less than 60 degrees. In embodiment nine (9), a device according to embodiment eight (8) has a connection angle equal to or less than 45 degrees.

A tenth embodiment (10) provides for a device from embodiments one through nine (1-9) wherein the outlet connection region further comprises a flow stream splitter portion. Embodiment eleven (11) is a device of embodiment ten (10) wherein the flow stream splitter portion protrudes into the processing channel and is constructed and arranged to separate the respective flow streams into their outlet channels.

Embodiment twelve (12) is a device according to embodiments one through eleven (1-11) wherein the first outlet channel comprises a first outlet collection channel and first outlet diversion channel. Embodiment thirteen (13) provides for a device of embodiment twelve (12) wherein the first outlet channel further comprises a valve constructed and arranged such that the flow stream in the first outlet channel is in selectable fluidic communication with the first outlet collection channel or the first outlet diversion channel.

Embodiment fourteen (14) is a device of embodiments one through thirteen (1-13) wherein the second outlet channel comprises a second outlet collection channel and second outlet diversion channel.

Embodiment fifteen (15) is a device of embodiment fourteen (14) wherein the second outlet channel further comprises a valve constructed and arranged such that the flow stream in the second outlet channel is in selectable fluidic communication with the second outlet collection channel or the second outlet diversion channel. Embodiment sixteen (16) provides for a device according to embodiments one through fifteen (1-15) wherein the fluidic device further comprises a magnet producing a gating magnetic field in the inlet channel or the inlet region. Embodiment seventeen (17) is a device of embodiment sixteen (16) wherein the magnet producing a gating magnetic field is an annular or toroidal magnet surrounding the inlet channel or inlet region. In embodiment eighteen (18), a device is provided according to embodiment sixteen (16) wherein the magnet producing a gating magnetic field is aligned and adjacent to the processing channel and extends to or beyond the channel inlet region of the inlet channel.

A nineteenth embodiment (19), a device of embodiments one through eighteen (1-18) comprises a processing channel that is optically transparent. In embodiment twenty, a device of embodiment one through eighteen (1-18) comprises an inlet channel that is optically transparent. Embodiment twenty-one provides for a device of embodiment one through twenty (1-20) further comprising an inlet channel flow controller a first outlet channel controller, a second outlet channel controller, or a combination thereof. Embodiment twenty-two (22) is a device according to embodiments B15-B18 further comprising a magnetic field controller (operatively linked to the annular or toroidal magnet).

Embodiment twenty-three (23) provides for a device of embodiments one through twenty-two (1-22) wherein the fluidic device comprises one or more sensors. Embodiment twenty-four (24) is a device of embodiment twenty-three (23) wherein the sensor is selected from an optical sensor, capacitive sensor, conductive sensor, thermal sensor, flowrate sensor, ultrasonic sensor, gravimetric sensor, magnetic field sensor, or combinations thereof. Embodiment twenty-five (25) is a device according to embodiment twenty-four (24) wherein the sensor is a photodetector, a multipixel imaging detector, a magnetic field detector, an electrochemical detector, an optical phase detector, a scatter detector, a Hall sensor, a magnetoresistive sensor, a bolometric sensor, surface acoustic wave sensor, a biosensor, or combinations thereof.

A twenty-sixth (26) embodiment provides a device of embodiments twenty-three to twenty-five (23-25) wherein a sensor is integrated into or adjacent to the processing channel. A twenty-seventh embodiment (27) provides a device of embodiments twenty-three to twenty-five (23-25) wherein a sensor is integrated into or adjacent to the inlet channel. A twenty-eighth embodiment (28) provides a device of embodiments twenty-three to twenty-five (23-25) wherein a sensor is integrated into or adjacent to one or more outlet channels. Embodiment twenty-nine (29) is a device of embodiments twenty-three to twenty-five (23-25) wherein the fluidic device comprises one or more sensors in or adjacent to the processing channel, one or more sensors in or adjacent to the inlet channel, one or more sensors in or adjacent to at least one outlet channel, or combinations thereof.

Embodiment thirty (30) is a device according to embodiments twenty-five to twenty-seven (25-27) further comprising an inlet channel flow controller wherein at least one sensor is operatively linked to an inlet flow controller. Embodiment thirty-one (31) is a device according to embodiments twenty-five to twenty-eight (25-28) further comprising an outlet channel flow controller wherein at least one sensor is operatively linked to an outlet flow controller. Embodiment thirty-two is a device according to embodiments twenty-three to twenty-nine (23-29) further comprising an annular or toroidal magnet surrounding the inlet channel or inlet region and a magnetic field controller wherein a sensor is operatively linked to the magnetic field controller to control the magnet field of the annular or toroidal magnet.

Embodiment thirty-two (32) is a device according to device embodiments one through thirty-one (1-31) wherein the inlet channel is further comprised of portion substantially linear to the processing channel and a portion that is substantially not linearly aligned to the processing channel connecting at a portion that is angled with an angle, theta (Θ), wherein Θ≠180° and Θ≥90°, is ≥100°, is ≥135°, is ≥140°, is ≥165°>180°, is ≥205°, is ≥225°, is ≥250°, or is ≤270° relative to the Y or Z-axis or relative independently to the Y- and Z-axis.

### Flowcell Cartridge Embodiments

An first embodiment (1) of a flowcell cartridge of the present invention comprising a planar substrate comprising an upper surface and a lower surface, a first longitudinal side forming an imaging surface, a second longitudinal side forming an illumination surface, and a first and second transverse side, an inlet well on an upper surface, an inlet channel, a sample processing channel in fluidic communication with the inlet channel and positioned substantially parallel to a longitudinal side, a sample splitter within the processing channel, a plurality of outlet channels in fluidic communication with the processing channel, and a plurality of collection wells in fluidic communication with each of the plurality of outlet channels wherein the substrate optionally comprises an optically transparent material and wherein the processing channel is offset within the plane of the of the substrate to be spatially biased to the imaging surface.

A second embodiment (2) of a flowcell cartridge of the present invention comprises a planar substrate comprising an inlet well on an upper surface, an inlet channel, a sample processing channel, a sample splitter withing the processing channel, a plurality of outlet channels in fluidic communication with the processing channel, and a plurality of collection wells in fluidic communication with each of the plurality of outlet channels wherein the substrate comprises an optically transparent material and wherein the combined volume each of the plurality of outlet channels is greater than the volume of the processing channel.

A third embodiment (3) is a flowcell cartridge according to embodiments 1 and 2 wherein the outlet channels follow compacted paths, one exemplary configuration being a serpentine channel.

A fourth embodiment (4) is a flowcell cartridge according to embodiments 1-3 wherein the outlet channels of the flowcell cartridge are formed as recesses within the planar substrate and a first outlet channel comprises a recess on a surface of the planar substrate and a second outlet channel comprises a recess on an opposite side of the planar substrate. In embodiments 1-4, the channels are formed by etching, machining, 3D printing, or molding the planar substrate.

A fifth embodiment (5) of the flowcell cartridge of embodiment 4 comprising one or more additional planar layers positioned over the recesses in the planar substrate to form enclosed channels.

A sixth embodiment (6) comprises the flowcell cartridge of embodiments 1-5, wherein the substrate is comprised of nonferrous metal, ceramic, glass, polymer, or plastic and, in the case of an embodiment with a substrate and one or more layers, the substrate and planar layer may be comprised of the same or different material.

A seventh embodiment (7) of the flowcell cartridge comprises embodiments 5-6 wherein the one or more planar layers are attached to the planar substrate by compression, adhesive bonding, preferably a biocompatible adhesive, more preferably a silicone or silicone-based adhesive, solvent bonding, ultrasonic welding, thermal bonding, welding, or 3D printing.

An eighth embodiment (8) of the flowcell cartridge comprises embodiments 5-7 wherein the planar substrate and the one or more planar layers are comprised of the same material.

A nineth embodiment (9) of the flowcell cartridge comprises the flowcell cartridge of embodiments 1-8 wherein the planar substrate comprises a polymer material.

A tenth embodiment (10) of the flowcell cartridge comprises the flowcell cartridge of embodiment 9 wherein the polymer material of embodiment 8 comprises cyclic olefin polymer or cyclic olefin copolymer.

An eleventh flowcell cartridge embodiment (11) comprises embodiments 1-10 and further comprises a collection well formed on the planar substrate and in fluidic communication with a terminal portion of an outlet channel.

A twelfth embodiment (12) of the flowcell cartridge of embodiments 1-11 wherein the collection well further comprises an internal channel inlet at a first well height and an internal outlet at a second well height wherein the inlet is in fluidic communication with an outlet channel of the flowcell cartridge and wherein the second well height is higher than the first well height.

A thirteenth embodiment (13) of the flowcell cartridge comprises the flowcell cartridges of embodiments 11-12 wherein the collection well further comprises a step providing an angled transition from a terminal aperture of the inlet to the collection well to the floor of the collection well.

A fourteenth embodiment (14) of the flowcell cartridges comprises the flowcell cartridge embodiments 11-13 further comprising a sealing film covering the top of one or more collection wells.

A fifteenth embodiment (15) of the flowcell cartridge comprises the flowcell cartridge embodiments 11-14 wherein the flowcell cartridge further comprises a collection well outlet channel in the planar substrate in fluidic communication with a collection well.

### Well-Based Embodiments

A first embodiment (1) of a well-based device or system for use with the Methods of the present invention, e.g., Method 6, comprises a fluidic concentrator device or system (System 1) for conducting magnetic levitation separation of samples in a multi-well plate, the system comprising:
a levitation sample fixture comprising:
a multi-well plate comprising a top surface and a plurality of wells, wherein said wells are optionally transparent;
a magnet array comprising a plurality of magnets disposed in between and below the wells, configured to provide a magnetic field in each of the wells;
a magnet holder configured to receive and hold the magnets of the magnet array;
optionally, a mirror assembly comprising:
   mirrors positioned to project, substantially parallel to the top surface of the plate, images of the wells along their vertical axes; and
   optionally a mirror holder;
optionally, a plurality of metal pins configured to attenuate and/or adjust the magnetic fields in the wells; and
optionally, a imaging array disposed beneath the multi-well plate and the magnet array.

### Embodiments of System 1

The following embodiments of System 1 are also useful in the practice of the Methods (e.g., Method 6) of the present disclosure:

System 1, further comprising a fluid transfer system.

System 1.1, wherein the fluid transfer system comprises means to dispense or remove all or part of a sample or other fluid into or out of the wells; e.g., a pipette or pipetting robot.

System 1, or 1.1-1.2, further comprising means to move the multi-well plate in a vertical direction relative to the magnet array.

Any System 1 or 1.1 et seq., wherein the magnet array comprises permanent magnets.

System 1.4, wherein for each well:
a first magnet and a second magnet are disposed adjacent to the well to impart a magnetic field that is inside the well; wherein each magnet comprises a first pole and a second pole, and the first pole of the first magnet faces the first pole of the second magnet; and
a third magnet is stacked under the first magnet, and a fourth magnet is stacked under the second magnet, wherein the third and fourth magnets each comprise a first pole and a second pole, and wherein the second pole of the third magnet faces the second pole of the fourth magnet.

System 1.5, wherein the magnets are rectangular magnets; optionally configured substantially in accordance with FIG. 35.

System 1.4, wherein the magnets are rectangular magnets arranged in a linear Halbach array.

System 1.4, wherein the magnets are ring magnets that surround the wells.

System 1.8, wherein the magnets are radially magnetized.

System 1.9, comprising for each well two ring magnets stacked one upon the other, wherein the poles of the stacked magnets are radially opposed.

System of claim 1.10, wherein the stacked ring magnets are configured to create a strong gradient at the interface with a low field zone in the height of the upper magnet.

System 1.8, wherein the magnets are configured to be axially magnetized.

Any System 1 or 1.1 et seq., wherein the magnets provide a magnetic field in each of the wells of between about 0.1 Tesla and about 2.0 Tesla and optionally between about 0.3 Tesla and about 1.0 Tesla at the surfaces of the magnets, varying to zero Tesla in certain locations due to superposition of fields from the plurality of magnets.

Any System 1 or 1.1 et seq., wherein the imaging array is present and comprises:
a) a microscope, e.g., a USB microscope and means for moving the microscope underneath the multi-well plate from well to well;
or
b) a camera array comprising a plurality of cameras; for example a motorized camera array.

System 1.14, wherein the plurality of cameras comprise one or more of built-in lenses, motorized focus, and zoom-in capability; and wherein each camera is configured to capture images from one to twenty-four of the wells; for example from four wells or from six wells.

System 1.14 or 1.15, further comprising a graphics processing unit (GPU) comprising graphics software that integrates two or more of the images from the cameras.

Any System 1 or 1.1 et seq., wherein the mirror assembly is present and comprises a mirror holder and mirrors.

System 1.17, wherein where the mirrors are either:
a) a separate layer with reflective surfaces; or
b) a mirror-coating disposed on the mirror holder.

Any System 1 or 1.1 et seq., wherein the mirror assembly is present and the mirrors are positioned at about 40° to about 50° with respect to the vertical axis substantially perpendicular to the main surface of the multi-well plate.

Any System 1 or 1.1 et seq., wherein the mirror assembly is present and the mirrors are configured substantially in accordance with FIG. 36.

Any System 1 or 1.1 et seq., wherein the wells are conical.

Any System 1 or 1.1 et seq., wherein the wells have four vertical sides and a square bottom.

Any System 1 or 1.1 et seq., wherein the levitation sample fixture comprises a plurality of metal pins configured to attenuate and/or adjust the shape of the magnetic fields in the wells.

System 1.23, wherein the metal pins are steel rods disposed in between the wells and are configured to increase the magnetic field gradients within the wells.

Any System 1 or 1.1 et seq., wherein the multi-well plate is configured to fit in the footprint of a standard 96-well PCR plate; or is in accordance with ANSI SLAS microplate standards; e.g., with a footprint of 127.76 mm×85.48 mm+0.5 mm.

Any System 1 or 1.1 et seq., wherein compatibility with standards or common equipment is achieved by setting the spacing between wells to 9 mm.

System 1.4, wherein for each well:
a first magnet and a second magnet are disposed adjacent to, and on opposite sides of, the well to impart a magnetic field that is inside the well; wherein each magnet comprises a first pole and a second pole, and the first pole of the first magnet faces the first pole of the second magnet;
a third magnet is stacked under the first magnet, and a fourth magnet is stacked under the second magnet, wherein the third and fourth magnets each comprise a first pole and a second pole, and wherein the second pole of the third magnet faces the second pole of the fourth magnet;
a fifth magnet and a sixth magnet are disposed adjacent to, and on opposite sides of, the well, to impart a magnetic field that is inside the well; wherein each magnet comprises a first pole and a second pole, and the first pole of the fifth magnet faces the first pole of the sixth magnet, and wherein the fifth and sixth magnets are oriented at approximately 90 degrees in the horizontal plane relative to the first and second magnets; and
a seventh magnet is stacked under the fifth magnet, and an eighth magnet is stacked under the sixth magnet, wherein the seventh and eighth magnets each comprise a first pole and a second pole, and wherein the second pole of the seventh magnet faces the second pole of the eighth magnet.

System 1.27, wherein the magnets are rectangular magnets; optionally configured substantially in accordance with FIG. 40B.

Any System 1.27 et seq., wherein the magnets provide a magnetic field in each of the wells of between about 0.1 Tesla and about 2.0 Tesla and optionally between about 0.3 Tesla and about 1.0 Tesla at the surfaces of the magnets, varying to zero Tesla in certain locations due to superposition of fields from the plurality of magnets.

Any System 1.27 et seq., wherein the imaging array is present and comprises:
a) a microscope, e.g., a USB microscope and means for moving the microscope underneath the multi-well plate from well to well;
or
b) a camera array comprising a plurality of cameras; for example a motorized camera array.

System 1.30 wherein the plurality of cameras comprise one or more of built-in lenses, motorized focus, and zoom-in capability; and wherein each camera is configured to capture images from one to twenty-four of the wells; for example from four wells or from six wells.

System 1.30 or 1.31, further comprising a graphics processing unit (GPU) comprising graphics software that integrates two or more of the images from the cameras.

Any System 1.27 et seq., wherein the mirror assembly is present and comprises a mirror holder and mirrors.

System 1.33, wherein where the mirrors are either:
a) a separate layer with reflective surfaces; or
b) a mirror-coating disposed on the mirror holder.

Any System 1.27 et seq., wherein the mirror assembly is present and the mirrors are positioned at about 40° to about 50° with respect to the vertical axis substantially perpendicular to the main surface of the multi-well plate.

Any System 1.27 et seq., wherein the mirror assembly is present and the mirrors are configured substantially in accordance with FIG. 36.

Any System 1.27 et seq., wherein the wells are conical.

Any System 1.27 et seq., wherein the wells have four vertical sides and a square bottom.

Any System 1.27 et seq., wherein the levitation sample fixture comprises a plurality of metal pins configured to attenuate and/or adjust the shape of the magnetic fields in the wells.

System 1.39, wherein the metal pins are disposed in between the wells, for example diagonally in between the wells, and are configured to increase the magnetic field gradients at selected area(s) within the wells.

System 1.39 or 1.40, wherein the metal pins are disposed substantially as shown in FIG. 26B.

Any System 1.39-1.41, wherein the metal pins comprise or are made of a material with high magnetic susceptibility such as steel, iron, cobalt, nickel, and alloys such as manganese alloy, magnesium alloy, nickel alloy, and chromium alloy, for example steel, manganese alloy, nickel alloy or chromium alloy rods.

Any System 1.27 et seq., wherein the multi-well plate is configured to fit in the footprint of a standard 96-well PCR plate; or is in accordance with ANSI SLAS microplate standards; e.g., with a footprint of 127.76 mm×85.48 mm+0.5 mm.

Any System 1.27 et seq., wherein compatibility with standards or common equipment is achieved by setting the spacing between wells to 9 mm, or any other standard or non-standard microplate or well plate layout.

Any System 1.1 et seq., wherein the fluid transfer system comprises a pipette or pipetting robot.

System 1.45, wherein the pipette or pipetting robot is equipped with gripper or manipulator arm.

System 1.45 et seq., wherein the pipettor tips are aligned with the centers of the sample wells.

System 1.47, wherein the pipetting robot comprises an optical alignment system.

System 1.48, wherein the optical alignment system is used to align or verify alignment of the pipettor tip(s) with the center of the sample well(s).

In further embodiments, Method 6 comprises isolation of a target subcellular component, e.g. cellular nuclei, from a sample comprising the target subcellular component and one or more contaminating species, using a System 1 or 1.1-1.48 as described above. In some such embodiments, the Method (Method 6A) comprises loading a sample comprising the target subcellular component, the contaminating species, and a sample medium comprising:
i) a paramagnetic compound or ferrofluid; and
ii) isolation particles or beads;

into a well of the System to form a sample fluid or sample suspension in said well; and

subjecting the sample fluid or sample suspension to a magnetic force from at least one magnet from the magnet array, to effect separation of the target subcellular component from the contaminating species; and

optionally imaging the sample fluid or sample suspension prior to, during, and/or after the separation.

The present disclosure further provides the following embodiments of Method 6A:
Method 6A, wherein the contaminating species is selected from one or more of a dissolved or suspended compound (e.g., a dye or dyes, antibodies, etc.); cellular debris; small particles (e.g., micro- or nano-beads); and dead cells.

Any preceding Method 6A, wherein the contaminating species is a dye or dyes.

Any preceding Method 6A, wherein the contaminating species is cellular debris.

Any preceding Method 6A, wherein the contaminating species is dead cells.

Any Method 6A, wherein the paramagnetic medium comprises one or more of a paramagnetic salt, a paramagnetic metal chelate, or a paramagnetic ionic liquid; for example a water soluble paramagnetic metal chelate.

Any Preceding Method 6A, further comprising collecting the target subcellular component.

Any Preceding Method 6A, wherein the separated target subcellular component is collected from the well in a pipette tip.

Any Preceding Method 6A, wherein the target subcellular component is collected by the steps of:
a) moving the multi-well plate vertically upward relative to the magnet array while maintaining the separated target subcellular component in place with the magnetic field, thereby causing the separated target subcellular component to migrate to the bottom of the well; and
b) either:
   i) removing the liquid above the separated target subcellular component with the pipette tip, leaving the separated target subcellular component in a reduced volume of fluid at the bottom of the well; or
   ii) removing the separated target subcellular component from the bottom of the well with the pipette tip.

Any Preceding Method 6A, wherein the separated target subcellular component are collected by the steps of:
a1) moving the pipette tip a vertical direction while maintaining the separated target subcellular component in place with the magnetic field, to bring the target subcellular component to the top of the pipette tip; and
a2) dispensing the liquid below the target subcellular component while retaining the separated target subcellular component in the pipette tip;

or
b1) moving the pipette tip a vertical direction while maintaining the separated target subcellular component in place with the magnetic field, to bring the separated target subcellular component to the bottom of the pipette tip; and
b2) selectively dispensing the separated target subcellular component, leaving unwanted liquid in the pipette tip.

Any Preceding Method 6A, wherein the separated target subcellular component is collected by the steps of:
a) inserting the pipette tip into the sample past the levitation position of the separated target subcellular component;
b) drawing the liquid up into the pipette tip while maintaining the separated target subcellular component in place with the magnetic field; and
c) dispensing just the separated target subcellular component.

Any Preceding Method 6A, wherein step (a) is performed early in the levitation process.

Any Preceding Method 6A, wherein the separated live cells are collected by the steps of:
a) after achieving levitation equilibrium, aspirating the liquid into the pipette tip until the separated target subcellular component is aspirated;
b) withdrawing the pipette tip from the well and immersing it into a second well in the plate or a separate plate that contains a washing liquid;
c) aspirating the separated live cells into the washing liquid while retaining the liquid in the pipette; i.e., slowly removing the pipette tip and the liquid therein, while the field maintains the position of the levitated target subcellular component, thus separating it from the original liquid; and
d) optionally repeating steps (a)-(c).

Any Preceding Method 6A, wherein the target subcellular component is levitated by radial magnets, and collected by the steps of:
a) aspirating the entire sample into the pipette tip;
b) moving the pipette tip and sample vertically upwards until they are clear of, and above, the magnets;
c) slowly lowering the tip into the magnetic field, and allowing a spheroid of particles to begin to coalesce in the region of minimal field;
d) after the tip completes passage through the region of minimal field, ejecting most of the tip's contents while the tip is moved slowly upwards, leaving a concentrated sample at the bottom of the tip; and
e) optionally transferring the concentrated sample.

Any Preceding Method 6A, wherein the target subcellular component is levitated by radial magnets, and collected by the steps of:
a) slowly aspirating the entire sample into the pipette tip creating a spheroid of particles at the region of minimal field;
b) moving the pipette tip vertically upwards through the magnetic field while the particle spheroid remains stationary relative to the field;
c) rapidly moving the sample to a destination, e.g., an output well; and
d) depositing a small volume containing the spheroid in the destination.

Any Preceding Method 6A, wherein the magnetic field strength at a surface of at least one of the magnets adjacent to the wells is between about 0.1 Tesla and about 2.0 Tesla and optionally between about 0.3 Tesla and about 1.0 Tesla

Any Preceding Method 6A, wherein the paramagnetic compound is present in the sample solution at a concentration of from about 20 mM to about 500 mM, optionally from about 50 mM to about 175 mM, and further optionally from about 70 mM to about 150 mM.

Any Preceding Method 6A, wherein the enriched recovered sample comprises at least about 60%, at least about 70%, at least about 80% or at least about 90% target subcellular component.

Any Preceding Method 6A, wherein the yield of target subcellular component in the enriched recovered sample fraction is at least about 50%, at least about 60%, at least about 70%, or at least about 75% of the total target subcellular component composition of the sample.

Any Preceding Method 6A, wherein the sample is loaded into the well or wells, and/or transferred out of the well or wells, using a pipetting robot.

Any Preceding Method 6A, wherein the magnetic levitation is monitored and/or recorded in real time by the visualization system.

Any Preceding Method 6A, further comprising removing the multi-well plate and replacing it with another multi-well plate.

Any Preceding Method 6A, wherein the contaminating species is a dye or dyes.

Any Preceding Method 6A, wherein the visualization system is used to adjust the parameters for transferring the sample into or out of the well.

Any Preceding Method 6A, wherein the target subcellular component comprises cellular nuclei.

Any Preceding Method 6A, wherein the target subcellular component consists essentially of cellular nuclei.

Any Preceding Method 6A, wherein the target subcellular component consists of cellular nuclei.

### Cell or Nuclei Separation System Embodiments

A first cell separation system embodiment (1) of the present invention comprises a receiving block for retaining a flowcell cartridge, an optical system comprising an optical sensor, a lens, and an illumination source, and plurality of flow modulation components, wherein the receiving block removably places the flowcell cartridge in optical alignment with the optical system, removably engages a magnetic component adjacent to the processing channel of the flow cell, and removably places a plurality of outlet channels of the flowcell cartridge in fluidic communication with the plurality of flow modulation components.

In a second embodiment (2), embodiment 1 further comprises a source of visible optical illumination constructed and arranged to provide light transmission through the processing channel within the planar substrate.

In a third embodiment (3), the system of embodiments 1-2 further comprise one or more sources of ultraviolet illumination constructed and arranged to place the ultraviolet illumination, optionally at wavelengths of about 474 nm and/or 560 nm, in an angular orientation to a processing channel within a planar substrate retained in the receiving block.

In a fourth embodiment (4) of the cell separation system comprising embodiment 3, the optical system comprises a dual bandpass filter preferably passing emitted radiation in bands centered at wavelengths at about 524 nm and 628 nm.

### BRIEF DESCRIPTION OF THE DRAWINGS

FIG. 1A-1D are cross-sectional views of embodiments of a single-magnetic component particle concentrating and isolating device as described herein.

FIG. 2A-2F are cross-sectional views of embodiments of a single-magnetic component particle concentrating and isolating device as described herein with angled inlet channel.

FIG. 3A-3D are cross-sectional views of embodiments of a single-magnetic component particle concentrating and isolating device as described herein with angled inlet channel and inlet magnet field component.

FIG. 4A-4D are cross-sectional views of embodiments of a particle concentrating and isolating device as described herein a magnetic component surrounding a portion of an inlet channel.

FIG. 5A-5D are cross-sectional views of embodiments of a single-magnetic component particle concentrating and isolating device as described herein with angled inlet channel and inlet magnet field component.

FIG. 6 depicts the magnetic field imposed within a processing channel in a single magnetic component configuration in accordance with an embodiment as described herein.

FIG. 7A-7D are cross-sectional views of embodiments of a multiple-magnetic component particle concentrating and isolating device as described herein with angled inlet channel and inlet magnet field component.

FIG. 8A-8D are cross-sectional views of embodiments of a particle concentrating and isolating device as described herein with angled inlet channel and inlet magnet field component. FIGS. 8E-H illustrate the incorporation of inlet channel valves controlling movement of fluid to optional intersecting channel(s).

FIG. 9A-9B are cross-sectional views of an embodiment of a particle concentrating and isolating device as described with selectable valves and inlet channel pump component.

FIG. 10 is a diagram of an integrated system according to various embodiments as described herein.

FIG. 11 depicts operation of single magnetic component embodiment of a particle concentrating and isolating device as described herein.

FIG. 12 depicts operation of an angled inlet channel embodiment of a particle concentrating and isolating device as described herein.

FIG. 13 depicts flow-enabled fractionation of a particle-containing sample according to operational embodiments of a particle concentrating and isolating device as described herein.

FIG. 14 depicts inlet channel magnetic field assisted concentration of a sample according to embodiments of a particle concentrating and isolating device as described herein.

FIG. 15 depicts inlet channel magnetic field assisted concentration of a sample according to embodiments of a particle concentrating and isolating device as described herein.

FIG. 16 depicts a meniscus facilitated concentration of a particle-containing sample according to operational embodiments of a particle concentrating and isolating device as described herein.

FIG. 17 is a micrograph of isolation of blood cells from a blood sample performed by an embodiment of a particle concentrating and isolating device as described herein.

FIG. 18 is a perspective view of an embodiment of a particle concentrating and isolating device as described herein.

FIG. 19A-19B are views of an example flowcell cartridge showing the imaging surface on the longitudinal side and the illumination surface on a second longitudinal side.

FIG. 20A-20B are detailed views of an example flowcell cartridge showing the top side and bottom side of the planar substrate

FIG. 21A-21B show example configurations of a collection well.

FIG. 22A-22B show an example of a system receiving block and the orientation of an example flowcell with respect to a plurality of magnetic components.

FIG. 23 shows an example of an optical system in orientation with an example receiving block.

FIG. 24 shows an image of an example particle separation and stability of outlet channel pressure.

FIG. 25A-25B show examples of collected cell viability and live cell yield

FIG. 26A-26B show examples of collected live cell purity and yield for various cell types.

FIG. 27 shows microscope images of unsorted (A, C) and sorted (B, D) nuclei derived from lung (A, B) and brain (C, D) tissue.

FIG. 28 shows flow cytometry analysis of unsorted (A) and sorted (B) nuclei derived from Jurkat cells.

FIG. 29 shows the levels of nuclear specific target (SCARNA5) and contaminating cytoplasmic target (SNHG6) in separated samples compared to input nuclei separated by the Levicell system.

FIG. 30 shows the effect of WGA on bulk nuclei isolation.

FIG. 31 shows images of the LeviCell flow cell during separation of nuclei with and without 3 micron streptavidin coated polystyrene beads.

FIG. 32 shows images from a cell counting system of the top and bottom fractions collected from the LeviCell.

FIG. 33 shows the spectrum data for the prestained nuclei with and without beads.

FIG. 34 shows Seurat reordered plots for the sequenced samples of nuclei.

FIG. 35 is a cross-sectional view of wells of a multi-well plate of a levitation sample fixture useful in the practice of the Methods of the invention showing one configuration of the magnets as described herein.

FIG. 36 is a cross-sectional view of a well of a multi-well plate of a levitation sample fixture useful in the practice of the Methods of the invention showing one configuration of the mirror as described herein.

FIG. 37 is an exploded view of a levitation sample fixture according to the present disclosure comprising mirrors.

FIG. 38 is an exploded view of a levitation sample fixture according to the present disclosure comprising metal pins.

FIG. 39 depicts the magnetic gradient of a Linear Halbach Array according to the present disclosure.

FIG. 40 depicts a comparison of the magnetic fields imposed by a linear array and a Halbach array within a well of a multi-well plate in accordance with an embodiment as described herein.

FIG. 41 depicts one configuration of radially magnetized ring magnets and their resulting magnetic field in accordance with an embodiment as described herein.

FIG. 42 is a depiction of a System in accordance with an embodiment as described herein.

FIG. 43 shows cross-sectional views of placement of magnets and mirrors in accordance with an embodiment as described herein.

FIG. 44 shows the effect of metal pins on the levitation of fluorescent polystyrene microparticles in accordance with an embodiment as described herein.

FIG. 45 shows cross-sectional views of placement of magnets and metal pins in accordance with an embodiment as described herein.

FIG. 46 depicts an exemplary workflow using the device of the present disclosure.

FIG. 47 shows the separation of beads by a System according to the present disclosure.

FIGS. 47a and 47b show the separation of a mixture of green and red beads (fluorescent polystyrene microparticles) having different density by magnetic levitation in a 96-well plate according to the present disclosure.

FIG. 48 depicts retrieval of levitated cells in accordance with an embodiment as described herein.

FIG. 49 depicts retrieval of levitated cells in accordance with a further embodiment as described herein.

FIG. 50 depicts retrieval of levitated cells in accordance with a further embodiment as described herein.

FIG. 51 depicts retrieval of levitated cells in accordance with a further embodiment as described herein.

FIG. 52 depicts retrieval of cells levitated with radial magnets in accordance with a further embodiment as described herein.

FIG. 53 depicts retrieval of cells levitated with radial magnets in accordance with a further embodiment as described herein.

FIG. 54 is a detailed view of a well plate design in accordance with an embodiment as described herein.

FIG. 55 is a detailed view of a well plate design containing mirrors in accordance with an embodiment as described herein.

FIG. 56 shows separation of live Jurkat cells in accordance with an embodiment as described herein.

FIG. 57 shows UMAPs of clustered cell-drug populations separated in accordance with an embodiment as described herein.

FIG. 58 shows a comparison of the clustering among cell-drug populations separated in accordance with an embodiment as described herein

FIG. 59 shows a magnetic array wherein four sets of magnets are disposed around each well, and the resulting magnetic levitation forces created thereby.

FIG. 60A shows the polarity of the magnets in magnetic arrays of the invention having two sets of magnets disposed around each well, and a magnetic array of the invention (designated “LV”) having four sets of magnets disposed around each well.

FIG. 60B shows an overhead view of wells of a multi-well plate of a levitation sample fixture of the invention wherein four sets of magnets are disposed around each well (designated “LV”), and the placement of optional metal pins.

FIG. 61 shows the washing of a cell sample by an immersion method of the present disclosure in accordance with Method 1.12.

FIG. 62 shows the magnetic field lines of an exemplary Dual Stacked Magnetic Array of the present disclosure and a LV Array of the present disclosure.

FIG. 63 shows a comparison of the particle height and response time for an exemplary Dual Stacked Magnetic Array of the present disclosure and a LV Array of the present disclosure.

FIG. 64 shows the results of debris and dead cell removal evaluated with FACS.

FIGS. 65A and 65B show the results of dead cell removal evaluated with FACS after manual wash and levitation wash, respectively.

FIG. 66 shows an exploded view of a LV Array of the present disclosure.

### DETAILED DESCRIPTION OF THE INVENTION

### I. Definitions/Nomenclature

The following definitions are provided to aid in understanding the invention. Unless otherwise defined, all terms of art, notations and other scientific or engineering terms or terminology used herein are intended to have the meanings commonly understood by those of skill. In some cases, terms with commonly understood meanings are defined herein for clarity and/or for ready reference, and the inclusion of such definitions herein should not be assumed to represent a substantial difference over what is generally understood in the art but is intended to compliment such general understandings. To the extent a definition herein is inconsistent with what is generally understood in the art, unless expressly stated otherwise, both the definition provided herein and what is generally understood in the art shall be deemed to be within the scope of the present invention as alternative embodiments.

As used herein unless otherwise indicated, open terms such as “contain,” “containing,” “include.” “including.” and the like mean comprising.

Some embodiments herein contemplate numerical ranges. When a numerical range is provided, the range includes the range endpoints unless otherwise indicated. Unless otherwise indicated, numerical ranges include all values and subranges therein as if explicitly written out.

As used herein, the article “a” means one or more unless explicitly stated otherwise.

Some values herein are modified by the term “about.” In some instances, the term “about” in relation to a reference numerical value can include a range of values plus or minus 10% from that value. For example, the amount “about 10” can include amounts from 9 to 11. In other embodiments, the term “about” in relation to a reference numerical value can include a range of values plus or minus 10%, 9%, 8%, 7%, 6%, 5%, 4%, 3%, 29%, or 1% from that value. Where a series of values is prefaced with the term “about.” the term is intended to modify each value included in the series.

As used herein, the term “asymmetric” about a magnetic field means that the magnetic field in the region of an associated fluidic channel is not symmetric about one or more planes passing through the center of the fluidic channel, and in accordance with a preferred embodiment it is not symmetric about the horizontal plane.

As used herein, the terms “capillary” or “capillary tube,” refer to a tube having a channel as defined hereinbelow.

As used herein, the terms “channel”, “flow channel,” “fluid channel” and “fluidic channel” are used interchangeably and refer to a pathway on a fluidic device in which a fluid can flow. Channel includes pathways with a maximum height dimension of about 100 mm, about 50 mm, about 30 mm, about 25 mm, about 20 mm, about 15 mm, about 10 mm, about 5 mm, about 5 mm, about 3 mm, about 2 mm, about 1 mm, or about 0.5 mm. The channel between magnets has dimensions of about 30 mm×0.5 mm, about 25 mm×1 mm, about 20 mm×2 mm, about 15 mm×3 mm, about 10 mm×5 mm, about 5 mm×3 mm, about 3 mm×2 mm, about 2 mm×1 mm, or about 1 mm×0.5 mm. For example, the channel between magnets has dimension of about 2 mm×1 mm. The internal height of the channel may not be uniform across its cross-section, and geometrically the cross-section may be any shape, including round, square, oval, rectangular, or hexagonal. The term “channel” includes, but is not limited to, microchannels and nanochannels, and with respect to any reference to a channel herein, such channel may comprise a microchannel or a nanochannel.

As used herein, the term “concentration” means the amount of a first component contained within a second component, and may be based on the number of particles per unit volume, a molar amount per unit volume, weight per unit volume, or based on the volume of the first component per volume of the combined components.

As used herein, the term “fluidically coupled” or “fluidic communication” means that a fluid can flow between two components that are so coupled or in communication.

As used herein, the terms “isolate” or “isolating” or “separate” or “separating” or “segregate” or “segregating” are used interchangeably, and they are in reference to a component means separating such component from other components, and includes increasing the concentration of a component within a solution, or separating a component from other components in a solution, or a combination of both increasing the concentration of a component within a solution while separating such component from other components in the solution. A particle within a solution is deemed “isolated” if it is segregated from other particles within the solution and/or positioned within a defined portion of the solution. A particle or component within a solution is also deemed “isolated” if after processing the solution the concentration of such particle or component is increased by a ratio of at least about 100:1, 90:1, 80:1, 70:1, 60:1, 50:1, 40:1, 30:1, 20:1, 10:1, 5:1, 3:1 or 2:1. Particles of interest within a solution containing other particles are deemed “isolated” if after processing such solution the ratio of the concentration of such particles of interest to the concentration of such other particles is increased, or if the ratio of the concentration of such particles of interest to the concentration of such other particles is increased by at least about 100%, 200%, 300%, 400%, 500%, 600%, 700%, 800%, 900%, or 1000%, or if the concentration of such other components is decreased to less than about 20%, 15%, 10%, 8%, 6%, 5%, 4%, 3%, 2%, 1%, or 0.5%.

As used herein, the term “fluidic” refers to a system, device or element for handling, processing, ejecting and/or analyzing a fluid sample including at least one “channel” as defined hereinabove. The term “fluidic” includes, but is not limited to, microfluidic and nanofluidic.

As used herein, the term “fluidic function” refers to any operation, function or process performed or expressed on a fluid or sample in a fluidic system, including, but not limited to filtration, pumping, fluid flow regulation, controlling fluid flow and the like.

As used herein, the term “particle” refers to any matter, including, but not limited to atoms, chemical elements, molecules, compounds, biomolecules, cells, necrotic cells, apoptotic cells, cancer cells, cancer or tumor circulating cells, cell nuclei, blood, plasma, proteins, lipids, bodily fluid, nucleic acids, nucleotides, amino acids, peptides, antibodies, antigens, carbohydrates, microorganisms, bacteria, viruses, fungi, sperms, gametes, eggs, embryos, or any physical substance with its largest dimension in any direction being less than about 3 mm, 2 mm, 1 mm, 0.5 mm, 0.25 mm, 100 microns, 75 microns, 50 microns, 40 microns, 30 microns, 20 microns, 10 microns, 5 microns, 2 microns, 1 micron, or 0.1 micron. The particle may have the largest dimension in any direction being about 0.001 micron to about 3 mm, about 0.1 micron to about 2 mm, about 0.5 microns to about 1.5 mm, about 10 microns to about 1 mm, or about 20 microns to about 100 microns.

As used herein, the term “isolation particle” means a particle or bead that a) forms a complex with the cellular debris, or form a structure in the sample medium that interacts with the cellular debris, in a manner that inhibits the movement of at least a portion of the cellular debris in a chosen direction relative to the movement of the cell nuclei; or b) forms a complex with the cellular debris, or form a structure in the sample medium that interacts with the cellular debris, in a manner to increase the movement of at least a portion of the cellular debris in a chosen direction relative to the movement of the cell nuclei. In some embodiments, the isolation particle is a bead as described herein, e.g. any of the bead type particles used for chromatographic separation, and can optionally be coated, for example with a protein. Examples of isolation particles include agarose beads, e.g. Sepharose™ beads, dextran beads e.g. Sephadex™, polyacrylamide beads, dextrose beads, polystyrene beads, beads made from polymeric resins. e.g. polyvinylethylcarbitol, polyvinylpyrrolidone, cellulose, silica-based materials, and/or from mixtures such as dextran-polyacrylamide, e.g. Sephacryl™ beads; each of which can optionally be coated, for example with a protein. In one particular embodiment, the isolation particles are polystyrene beads or particles; e.g. streptavidin coated polystyrene beads or particles, such as SPHERO™ Streptavidin Coated Particles sold by Spherotech, Inc., Lake Forest IL. In some embodiments, each of the foregoing particles can optionally comprise a ferromagnetic material to become susceptible to magnetism e.g. ferromagnetic particles, and can optionally be coated, for example with a protein. The sizes recited for isolation particles represent the largest dimension in any direction. For spherical isolation particles, the recited size represents the diameter.

As used herein, the term “port” refers to a structure for providing fluid communication between two elements using, for example, a fluidic channel. The terms “inlet port” or “inlet opening” or “input opening” or “input channel” are used interchangeably, and they refer to the opening where sample fluid is injected into the device described herein.

As used herein, the term “concentrate” or “concentrating” refers to making a substance in a medium with increased population density or purer by removing water, aqueous or non-aqueous medium or other substances. The substance is a type of particle or a mixture of particles as described herein. Typically, concentrating as described herein involves facilitated sedimentation of the particles or a mixture of particles in a medium, thereby bringing the particles or a mixture of to a particular area. Alternatively, concentrating may involve separating a particular type of particle from a mixture of particles and collecting the particular type of particle in a collecting channel, typically with pre-determined volume of a liquid medium. The concentrating need not involve spinning or rotating the bulk sample in order to concentrate the particles. Concentration performed by the present invention allows of separation of particles without significant damage, lysis, or shearing of the particles. Additionally, under certain conditions of operation, the present invention provides for flocculation or crystallization within a sample during operation and isolation of the flocculated or crystallized particles of the sample.

Where methods and steps described herein indicate certain events occurring in certain order, those of ordinary skill in the art will recognize that the ordering of certain steps may be modified and that such modifications are in accordance with the variations of the invention. Additionally, certain steps may be performed concurrently in a parallel process when possible, as well as performed sequentially.

### II. Magnetic Field

The present disclosure provides for methods and devices for concentrating using a magnetic field within a processing channel or inlet channel. The interaction of the magnetic field with the paramagnetic properties of particles within a sample fluid can either provide a repulsive or attractive effect on the particles to facilitate separation or concentration.

In accordance with an embodiment, magnets are permanent magnets or electromagnets. In accordance with an embodiment, the maximum energy product of magnets range from about 1 Mega-Gauss Oersted to about 1000 Mega-Gauss Oersted, and more preferably ranges from about 10 Mega-Gauss Oersted to about 100 Mega-Gauss Oersted. In accordance with an embodiment, the surface field strength of magnets range from about 0.1 Tesla to about 100 Tesla, and more preferably ranges from about 1 Tesla to about 10 Tesla. In accordance with an embodiment, the remanence of magnets range from about 0.5 Tesla to about 5 Tesla, and more preferably ranges from about 1 Tesla to about 3 Tesla.

In accordance with a preferred embodiment, magnets are made from a material comprising neodymium alloys with iron and boron, neodymium, alloys of aluminum with nickel, neodymium alloys with iron, aluminum and cobalt alloyed with iron, samarium-cobalt, other alloys of rare earth elements with iron, alloys of rare earth alloys with nickel, ferrite, or combinations thereof. In accordance with an embodiment comprising a plurality of magnets, magnets are made from the same material or are made from different materials.

In accordance with an embodiment, an asymmetric magnetic field is achieved by using a stronger magnetic material on one side of a fluidic channel and a weaker magnetic material on the opposite side of the fluidic channel. In accordance with a preferred embodiment, an asymmetric magnetic field is achieved by using a magnetic material on one side of a fluidic channel and a substantially similar magnetic material on the opposite side of the fluidic channel. In accordance with such embodiment, upper magnet and lower magnet may be substantially the same size. In accordance with such embodiment, upper magnet may comprise neodymium, lower magnet may comprise samarium-cobalt, and wherein both magnets are substantially the same size. Alternatively, upper magnet may comprise samarium-cobalt, lower magnet may comprise neodymium, and wherein both magnets are substantially the same size.

In accordance with an embodiment, alternative magnet configurations may be used. Referring to, the device in accordance with the present invention may include multiple upper magnets and multiple lower magnet positioned around a fluidic channel. Upper magnets may include an anterior upper magnet, a central upper magnet, and a posterior upper magnet. Lower magnets may include an anterior lower magnet, a central lower magnet, and a posterior lower magnet.

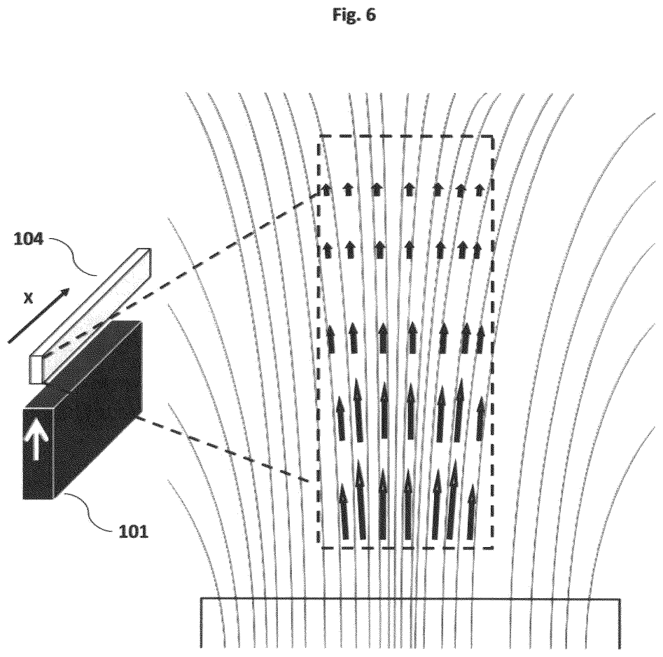

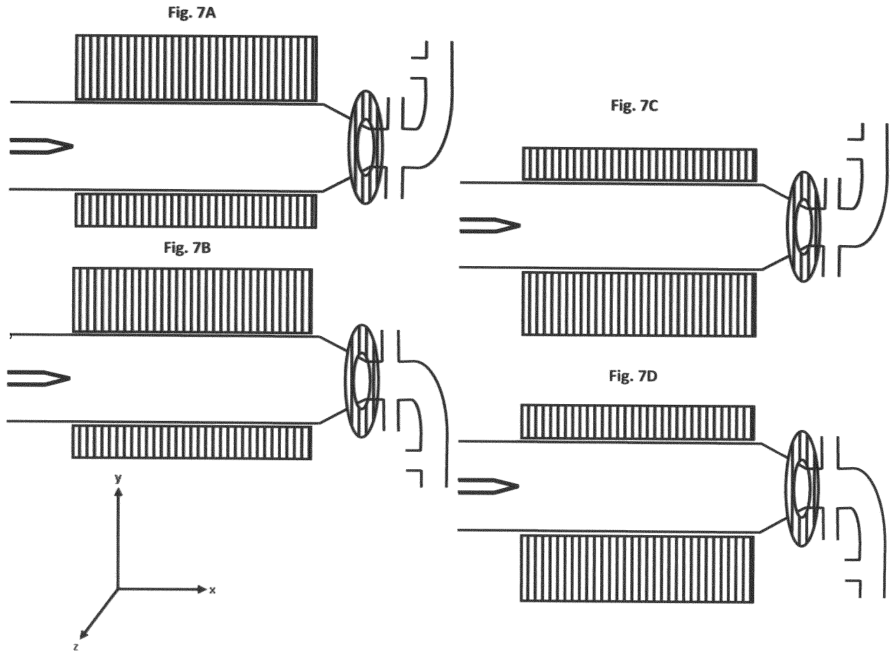

In accordance with another magnet configuration, the device may include an anterior upper magnet, a posterior upper magnet, an anterior lower magnet, and a posterior lower magnet, wherein the magnets are positioned around fluidic channel. The anterior upper magnet and the posterior lower magnet are positioned in a magnetic repelling orientation. Exemplary NdFeB magnetic component dimensions include, for a bottom magnet component about 50×15×2 mm (magnetized through the 15 mm axis), 50×5×2 mm (magnetized through the 5 mm axis) for a top magnet component. Other magnet component embodiments include 60×15×2 mm, 60×5×2 mm, 75×20×3 mm, and 25×15×2 mm. FIG. 6 illustrates an embodiment with a rectangular magnet substantially aligned along the X-axis at the bottom of processing channel 104 showing magnetic field lines and magnetic force within the processing channel. An additional preferred magnet component embodiment include an upper and lower magnet with dimensions of about 75×20×3.2 mm, and a spacing between upper and lower magnets of about 2.5 mm, about 3.0 mm, about 3.5 mm, about 2.9 mm, about, 3.0 mm, about 3.1 mm, about 3.2 mm, about 3.3 mm, or about 2.72 mm, about 2.88 mm, about 2.98 mm, about 3.18 mm, about 3.20 mm, or about 3.37 mm.

In a preferred embodiment the device has an upper magnet and a lower magnet wherein the flower magnet extends into the inlet channel. The bottom magnet dimensions can be about 50 mm to about 100 mm×about 10 mm to about 30 mm×about 2 mm to about 4 mm. An preferred embodiments include about 75 mm, about 80 mm, about 85 mm, about 90 mm, about 93 mm, or about 95 mm×about 15 mm, about 18 mm, about 20 mm, about 23 mm, and about 25 mm×about 2 mm, about 2.3 mm, about 2.5 mm, about 2.7 mm, about 3 mm, about 3.18 mm and about 3.5 mm. Magnet spacing between upper and lower magnets preferably is between 2 to 4.3 mm, about 2.5 mm, about 4.0 mm, about 3.5 mm, about 2.9 mm, about, 3.0 mm, about 3.1 mm, about 3.2 mm, about 3.3 mm, or about 2.72 mm, about 2.88 mm, about 2.98 mm, about 3.18 mm, about 3.20 mm, about 3.37 mm, about 3.5 mm, about 3.7 mm, or about 4 mm.

Embodiments of the fluidic concentrating device incorporating parallel magnet components on top and bottom and substantially aligned along the X-axis of the processing channel are illustrated in FIG. 7A-D.

### III. Paramagnetic Medium

Samples processed by magnetic facilitated concentration by the present invention will typically have an added paramagnetic component or an added diamagnetic component. In accordance with the method of the present invention, a substance containing particles of interest is combined with a paramagnetic medium to create a processing solution. The paramagnetic medium comprises a paramagnetic material and a solvent. In accordance with a preferred embodiment, the paramagnetic medium is biocompatible, i.e. capable of being mixed with live cells and not impact the viability of the cells or impacting cellular behavior, e.g. impacting gene expression. The paramagnetic material may be selected from the group comprising gadolinium, titanium, vanadium, dysprosium, chromium, manganese, iron, nickel, gallium, including ions thereof and combinations thereof. In accordance with an embodiment the paramagnetic material is selected from the group comprising titanium (III) ion, gadolinium (III) ion, vanadium (I) ion, nickel (II) ion, chromium (III) ion, vanadium (III) ion, dysprosium (III) ion, cobalt (II) ion, and gallium (III) ion. In accordance with a preferred embodiment, the paramagnetic material comprises a chelated compound. In accordance with a preferred embodiment, the paramagnetic material comprises a gadolinium chelate, a dysprosium chelate, or a manganese chelate. In accordance with an embodiment, the paramagnetic medium comprises a paramagnetic material, salts, and other additives that function to maintain cellular integrity. In an embodiment of the invention the paramagnetic material may be [Aliq]₂ [MnCl₄], [Aliq]₃ [GdCl₆], [Aliq]₃ [HoCl₆], [Aliq]₃ [HoBro], [BMIM]3 [HoCl₆], [BMIM] [FeCl₄], [BMIM]₂ [MnCl₄], [BMIM]₃ [DyCl₆], BDMIM]₃ [DyCl₆], [AlaC1] [FeCl₄], [AlaC1]₂ [MnCl₄], [AlaC1]₃ [GdCl₆], [AlaC1]₃ [HoCl₆], [AlaC1]₃ [DyCl₆], [GlyC2] [FeCl₄] as described in U.S. patent application Ser. No. 14/407,736 which is incorporated herein by reference.

In accordance with an embodiment, the paramagnetic material may be present in the paramagnetic medium at a concentration of at least about 10 mM, 20 mM, 30 mM, 40 mM, 50 mM, 60 mM, 70 mM, 80 mM, 90 mM, 100 mM, 120 mM, 150 mM, 200 mM, 250 mM, 300 mM, 500 mM, or 1 M. In accordance with an embodiment, the paramagnetic material may be present in the paramagnetic medium at a concentration of about 10 mM to about 50 mM, about 25 mM to about 75 mM, about 50 mM to about 100 mM, about 100 mM to about 150 mM, about 150 mM to about 200 mM, about 200 mM to about 250 mM, about 250 mM to about 300 mM, about 300 mM to about 500 mM, or about 500 mM to about 1 M.

In accordance with an embodiment, the paramagnetic material comprises gadolinium and is present in the paramagnetic medium at a concentration of at least about 10 mM, 20 mM, 30 mM, 40 mM, 50 mM, 60 mM, 70 mM, 80 mM, 90 mM, or 100 mM. In accordance with an embodiment, the paramagnetic material comprises gadolinium and is present in the paramagnetic medium at a concentration of about 10 mM to about 50 mM, about 25 mM to about 75 mM, or about 50 mM to about 100 mM.

### IV. Device Configuration

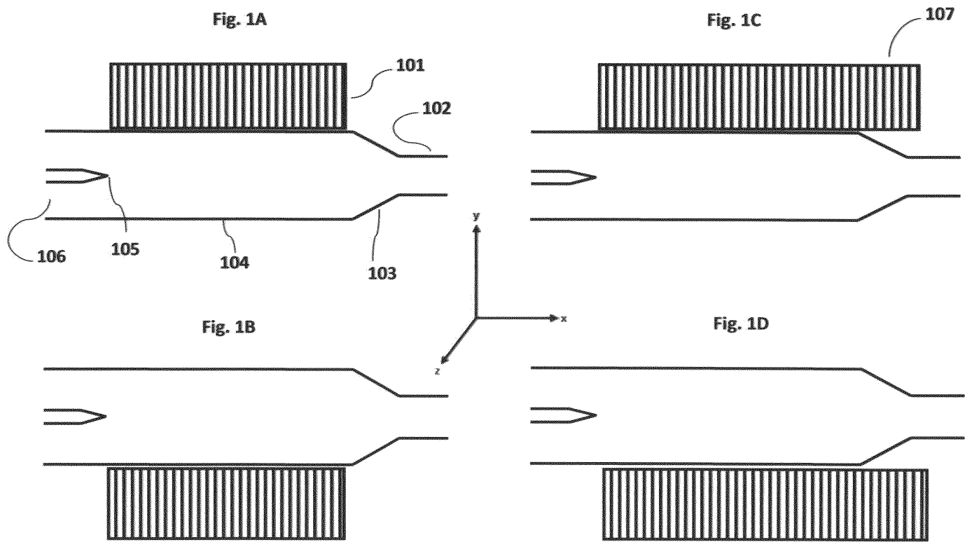

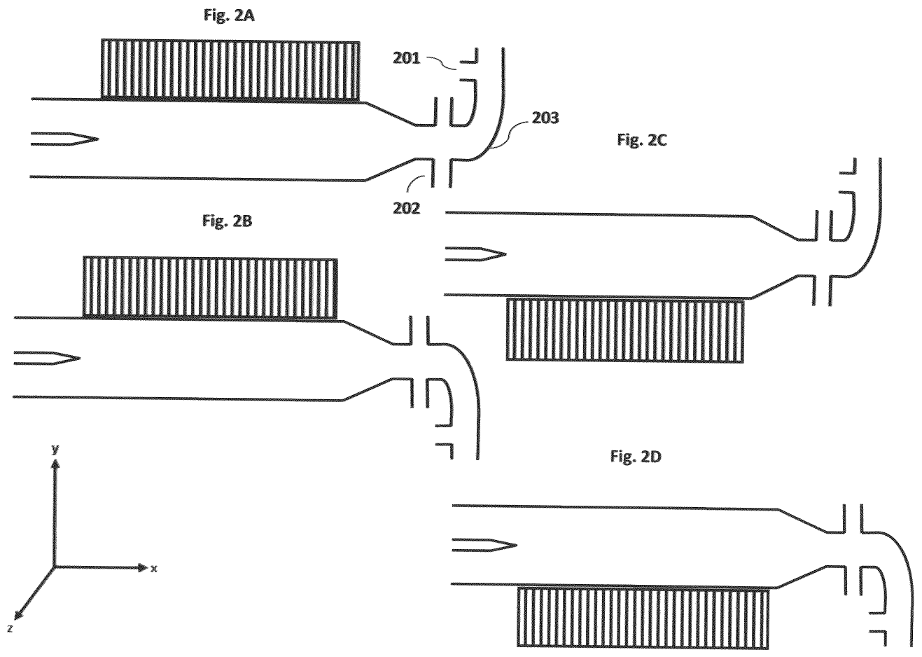

Referring to FIGS. 1 to 5, and 7-10, various embodiments of a particle concentration device of the present invention for particle concentrating and isolating are shown wherein the device's inlet channel, processing channel, and outlet channels portion comprise separate individual components that are interconnected. The processing channel is preferably an elongated fluidic channel that has sufficient length along the x-axis to allow sufficient time for processing a fluid containing the particles of interest based on the residence time required for the particles to concentrate within a layer of the flow stream (where “layer” in this sense indicates a small range of positions along the y-axis) within the processing channel, and based on the desired throughput from the system. In some embodiments, the processing channel is a fluidic channel that has a height of about 200 microns to about 30 mm, about 200 microns to about 20 mm, about 200 microns to about 15 mm, about 200 microns to about 10 mm, about 200 microns to about 5 mm, about 200 microns to about 2 mm, about 200 microns to about 1 mm, about 0.5 mm to about 1 mm, about 0.5 mm to about 2 mm, about 0.5 mm to about 3 mm, about 1 mm to about 2 mm, about 1 mm to about 3 mm, or about 1.5 mm to about 2 mm. In accordance with an embodiment, the processing channel has a length of about 20 mm to about 200 mm, about 20 mm to about 150 mm, about 20 mm to about 100 mm, about 20 mm to about 50 mm, about 40 mm to about 100 mm, about 40 mm to about 90 mm, or about 40 mm to about 80 mm. For some embodiments, the channel depth (in the Z direction) is about 100 microns to about 5 mm, about 500 microns to about 3 mm, about 1 mm to about 2.5 mm, or about 1.5 to about 2 mm. Channel length embodiments may be 40, 45, 50, 60, 70 mm in length. Dimensions of exemplary processing channels include 1 mm and 1.9 mm tall (Y-axis, vertical in images)×0.8 or 1.0 or 1.5 or 2.0 mm deep (Z-axis), with length (X-axis) 40-70 mm. A preferred embodiment of the processing channel has dimensions of exemplary processing channels include about 2 mm tall (Y-axis, vertical in images)×about 2.0 mm deep (Z-axis), with length (X-axis) about 50-70 mm. Further preferred processing channel embodiments have a length of about 55 mm, about 56, mm, about 57 mm, about 58 mm, about 59 mm, about 60 mm, about 61 mm, about 62 mm, about 63 mm, about 64 mm, about 65 mm, or about 66 mm.

The processing channel may have any cross-sectional geometric configuration and may comprise a cross sectional geometric configuration that is square, rectangular, round or oval. The geometric characteristics of processing channel described herein are equally applicable to the inlet, outlet, and any other fluidic channel described above in reference to the component configuration of the present invention.

The cross-sectional area of the inlet channel (which would be πr² where r is the radius of the inside diameter of the channel when the channel is circular) is substantially smaller than the cross-sectional area of the processing channel. In this context, “diameter” is used to describe a characteristic cross-sectional dimension, and the channel may not be circular in cross-section. In various embodiments, the cross-sectional area of the inlet channel is at least 100 times, 80 times, 50 times, 40 times, 20 times, 10 times, 8 times, 6 times, 4 times, or 2 times smaller than the cross-sectional area of the processing channel. In some embodiments, the cross-sectional area of the inlet channel is at least 10 times smaller than the cross-sectional area of the processing channel. In some embodiments, the cross-sectional area of the inlet channel is at least 5 times smaller than the cross-sectional area of the processing channel. In some embodiments, the cross-sectional area of the inlet channel is at most 0.2 mm², at most 0.8 mm², at most 3.1 mm², at most 7.1 mm², at most 12.6 mm², at most 19.6 mm², at most 28.3 mm², at most 38.5 mm², at most 50.3 mm², at most 78.5 mm², at most 176.7 mm², or at most 314.2 mm². Outlet channels typically have similar dimensional characteristics as the inlet channel but may have varying cross-sectional areas as described below.

Embodiments of the particle concentrating device include a tapered inlet portion to the processing channel (103) the particle isolation device of the present invention also includes a tapered entry port to reduce turbulent flow caused by vortices and thus reduce associated shear forces associated with the connection of fluidic channels of disparate cross-sectional areas. These vortexes may reduce the efficiency or rate of processing a sample, by providing locations where cells or other particles can be trapped in a circulating path, rather than flowing through the device. Vortex flow may also induce shear stresses on particles such as cells. The angle of taper may be between about 10° to about 70°, preferably between about 20° to about 60°, between about 30° to about 45°, or, in some embodiments, about 30°.

The outlet portion of the device may comprise a splitter that aids in diversion of portions of the sample stream in the processing channel into discrete streams for isolation or further processing. The splitter is preferably positioned within the processing channel, but near the trailing end of the processing channel, so that any particle isolation achieved by passing the fluid through the processing channel is maintained as the fluid exits the device. The splitter may comprise one or more horizontal partitions that extend from the outlet channels into the trailing end of the processing channel. The splitter may extend into the processing channel with a length between 0.5-3.5×, 1-3×, 1.5-2.5×, 1-2×, or 2× the distance between magnets on embodiments with magnets aligned substantially along the X-axis and on opposite sides of the processing channel. For singe magnet embodiments of the particle concentration device, the splitter may extend into the processing channel 1-5×, 1.5-5×, 1.5-4×, 2-4×, 3-4×, or 4× the thickness of the single magnet component in the Z-axis direction. For embodiments with no magnet component aligned along the X-axis of the processing channel, the splitter may extend between 5-40%. 5-30%, 5-25%, 10-30%, 10-20%, or 10-15% of the processing channel length, preferably greater than 5%, preferably less than 35%. The splitter tapers to point at the terminal end within the processing channel. The angle of taper maybe between about 5° to about 45°, preferably between about 10° to about 30°, between about 15° to about 25°, or, in some embodiments, about 20°. The processing channel may be horizontally divided using the splitter. In addition, the splitter may include one or more vertical partitions, thereby creating a horizontal and vertical grid of effluent fluidic outlet openings in fluidic communication leading to a plurality of outlet channels. In this embodiment, the plurality of outlet channels near the trailing end of the processing channel lead through outlet ports a plurality of collecting chambers, such as collecting tubes or Eppendorf tubes. The splitter(s) defines a plurality of outlet channels. In accordance with an embodiment, the particle concentration device of the present invention includes a splitter that defines 2, 3, 4, 5, 6, 7, 8, 9 or 10 outlet channels. In accordance with an embodiment, the particle concentration device of the present invention includes a splitter that defines at least 2, at least 3, at least 4, at least 5, at least 6, at least 7, at least 8, at least 9, or at least 10 outlet channels. In accordance with an embodiment, the particle concentration device of the present invention includes a splitter that defines 2 to 4, 5 to 7, or 8 to 10 outlet channels. The splitter described herein, and the various resulting outlet channels, may be incorporated into the processing channel described above in reference to the component configuration of the present invention.

The plurality of outlet channels extend from the processing channel to a corresponding plurality of outlet ports. The plurality of outlet channels may include fluidic flow modulators, such as pumps or output valves, that control the amount of flow from processing channel through the respective effluent channels to the respective outlet ports. The division of sample solution into each effluent fraction may be achieved by increasing or decreasing the fluid flow toward individual outlets, such that the ratio of division can be modified. In accordance with an embodiment the ratio may be modified by up to 50%. For example, if the splitter comprises two channels with equal cross-section, the geometric ratio of division is 1:1. By withdrawing a larger (or smaller) amount of fluid into one fraction through the application of a larger (or smaller) pumping rate than is applied to the other fraction, the ratio of division can be altered, e.g. to about 3:1, 4:1, 5:1, 6:1, 7:1, 8:1, 9:1 or 10:1. In a preferred embodiment, the division for such geometric ratio would be within the range of about 2:1 to about 1:2. In a further preferred embodiment, the division for such geometric ratio would be within the range of about 10:1 to about 1:10. The division could be about 1:10, about 1:9.5, about 1:9, about 1:8.5, about 1:8, about 1:7.5, about 1:7, about 1:6.5, about 1:6, about 1:5.5, about 1:5, about 1:4.5, about 1:4, about 1:3.5, about 1:3, about 1:2.5, about 1:2, about 1:1.5, about 1:1, about 1.5:1, about 2:1, about 2.5:1, about 3:1, about 3.5:1, about 4:1, about 4.5:1, about 5:1, about 5:5, about 5.5:1, about 6:1, about 6.5:1, about 7:1, about 7.5:1, about 8:1, about 8.5:1, about 9:1, about 9.5:1, about 10:1 each ratio respective of an upper outlet channel flowrate to a lower channel flowrate.

FIG. 1A illustrates a device with a single magnet component (101) substantially aligned along the top X-axis of processing channel (104), an inlet channel (102) and an inlet connection region (103), a plurality of outlet channels (106) and a flow stream splitter portion (105). FIG. 1B illustrates a similar configuration with 101 aligned along the bottom of the processing channel. FIG. 1C illustrates the configuration in 1A with an additional magnet component or portion (107) extending beyond the processing channel and into the inlet portion. FIG. 1D illustrates this type of configuration with a magnet component substantially aligned along the X-axis of the bottom of the processing channel and extending into the inlet portion. In accordance with an embodiment, one or both of the upper magnet and the lower magnet are movably mounted within the system to allow controlled adjustment of the vertical position of the magnet relative to fluidic channel and thereby adjust magnetic field strength within the channel. The use of inlet channel geometry and inlet channel magnetic field are used in certain embodiments of the invention to modulate particle concentration within the processing channel. By accumulating particles at or near the inlet of the processing channel, certain embodiments provide the ability to create portions of the flow stream that vary in their relative enrichment of particles. For example, accumulation of particles at or near the inlet of the processing channel may result in the instant depletion of quantity of particles in the flow stream within the processing channels. By releasing or surmounting the particle restraining force, sedimentary, magnetic or a combination thereof, the particle concentration within the flow stream in the processing channel is then increased. Temporally, this may result in varying levels of particle enrichment within the particle enriched layer of the flow stream in the processing channel. This selective process is useful, in one aspect, in creating flow stream fractions of particular interest to the user of the device and methods of the invention who can identify and utilize the particularly enhanced (enriched) or particularly depleted flow stream fractions.

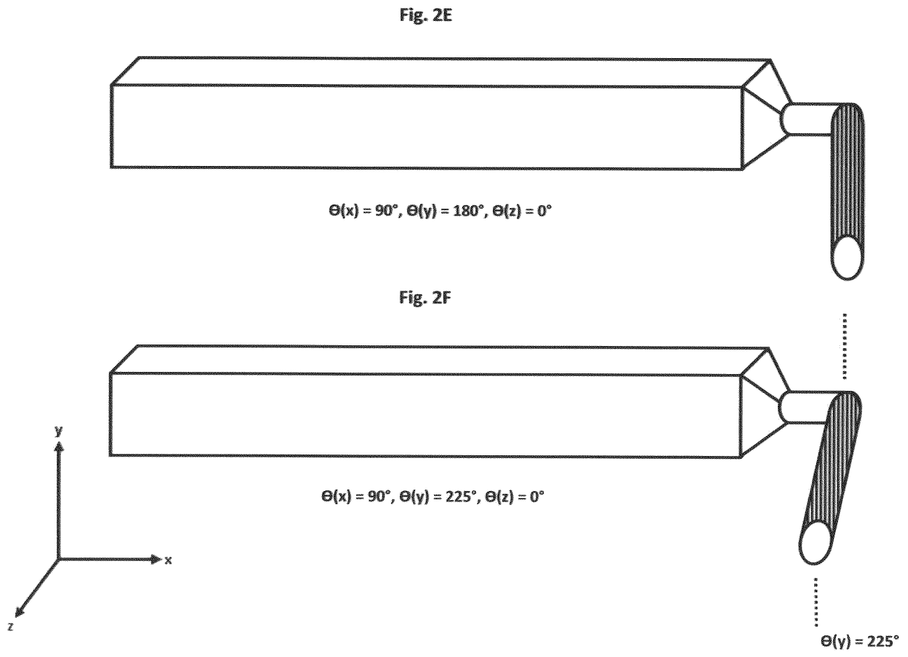

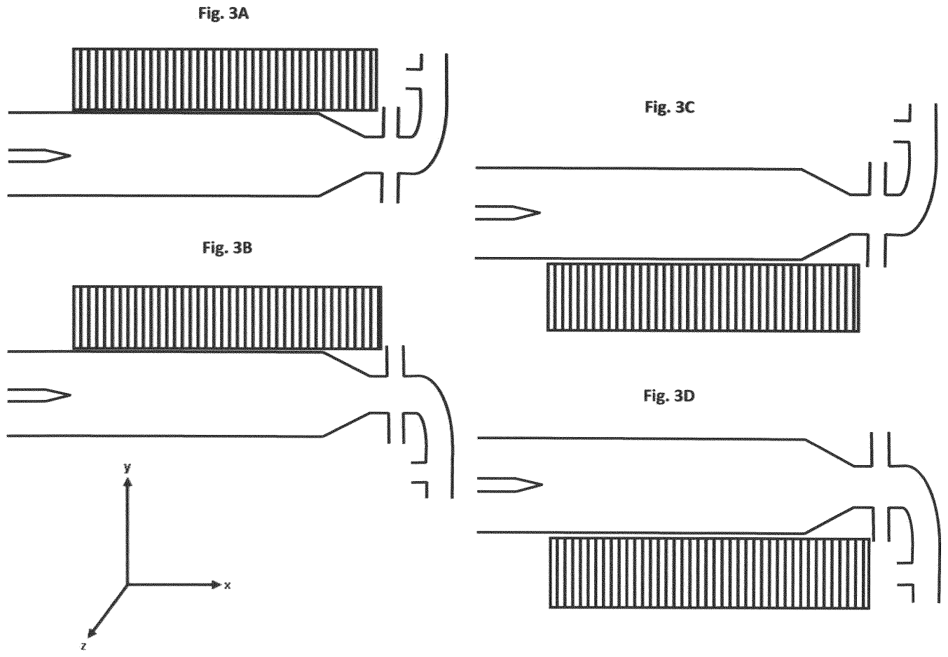

FIGS. 2A-D illustrate a single magnet component configuration further comprising an inlet channel portion (203) with a portion substantially linearly aligned to the processing channel and a portion that is not linearly aligned to the processing channel connecting at a portion that is angled with an angle, theta (Θ). The nonlinear inlet portion may contain one or more intersecting channels (201) and the linear inlet portion may comprise one or more intersecting channels (202) The intersecting channels to the inlet channel provide for the introduction or removal of liquids, suspensions, or gases. Intersecting channels can provide for reagent introduction into a sample flow stream such as paramagnetic medium, buffer, flocculating agent, sample preconditioning reagent or reactant, and the like. The intersecting channels may also be used to introduce reactant reagents into the sample medium for reaction with particles or other components of the sample medium within the inlet channel or within the processing channel. For example, a cell staining reaction or ligand binding reaction can be performed in the inlet channel prior to separation. Additionally, the reaction can, in some embodiments, be performed or continued in the processing channel if necessary or desired as might be the case with a precipitation, flocculation, or crystallization reaction. One or more of the intersecting channels can also be used as an output to collect particles repelled or otherwise prevented from entering the separation channel. FIG. 2A illustrates a fluidic concentrating device with a magnet component substantially aligned along the X-axis at the top of the processing channel. The nonlinear portion of the inlet channel is at about 90° relative to the substantially linear portion of the inlet channel. FIG. 2B illustrates a similar embodiment with the magnet component aligned substantially linear at the bottom of the processing channel and the nonlinearly aligned portion of the inlet channel is at about 270° relative to the substantially linear portion of the inlet channel. FIG. 2C illustrates an embodiment as in FIG. 2A with the magnet component aligned along the bottom of the processing channel. FIG. 2D illustrates an embodiment similar to FIG. 2B with the magnet component aligned along the bottom of the processing channel. In the embodiments in FIG. 2A-D, the nonlinearly aligned inlet channel portion is at about either 90° (FIG. 2B, 2D) or at about 270° (FIGS. 2A, 2C) relative to the substantially linear portion of the inlet channel, the angle Θ can be determined by device requirements wherein Θ≠0°, Θ≠180° and Θ≥30°, Θ≥45°, Θ≥70°, Θ≥90°, is ≥100°, is ≥135°, is ≥140°, is ≥165°>180°, is ≥205°, is ≥225°, is ≥250°, ≥280°, ≥300°, or is ≤330° relative to the X-axis and/or any angle, theta (Θ), relative independently to the Y- and Z-axis. For example, FIG. 2E illustrates a plan view where Θ(x)=90°, Θ(y)=180°, Θ(z)=0°. FIG. 2F illustrates a plan view where Θ(x)=90°, Θ(y)=225°, Θ(z)=0°.

FIG. 3A-D illustrate embodiments similar to those of FIG. 2A-D wherein a magnet component 107 extends into the inlet region.

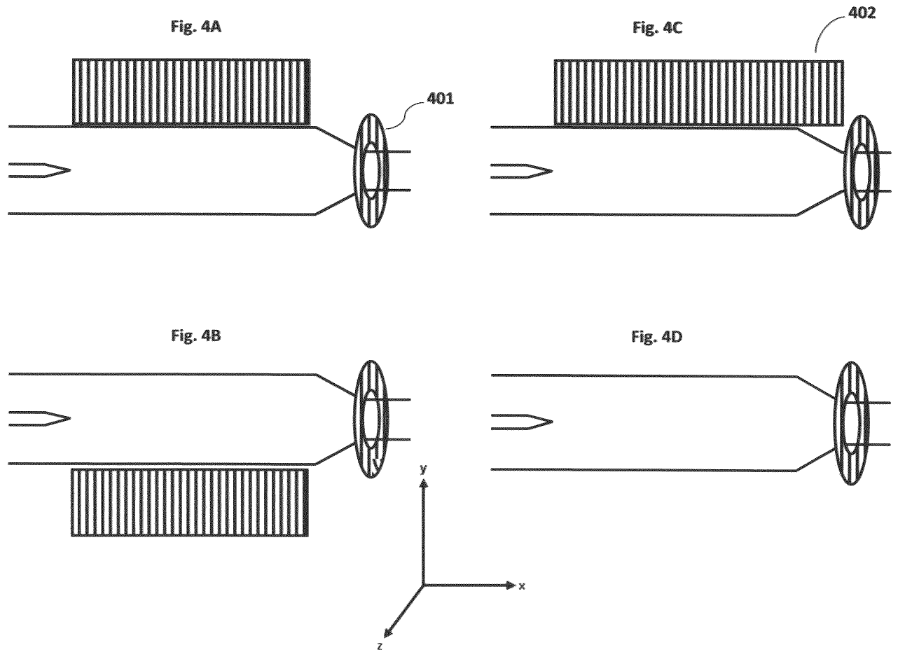

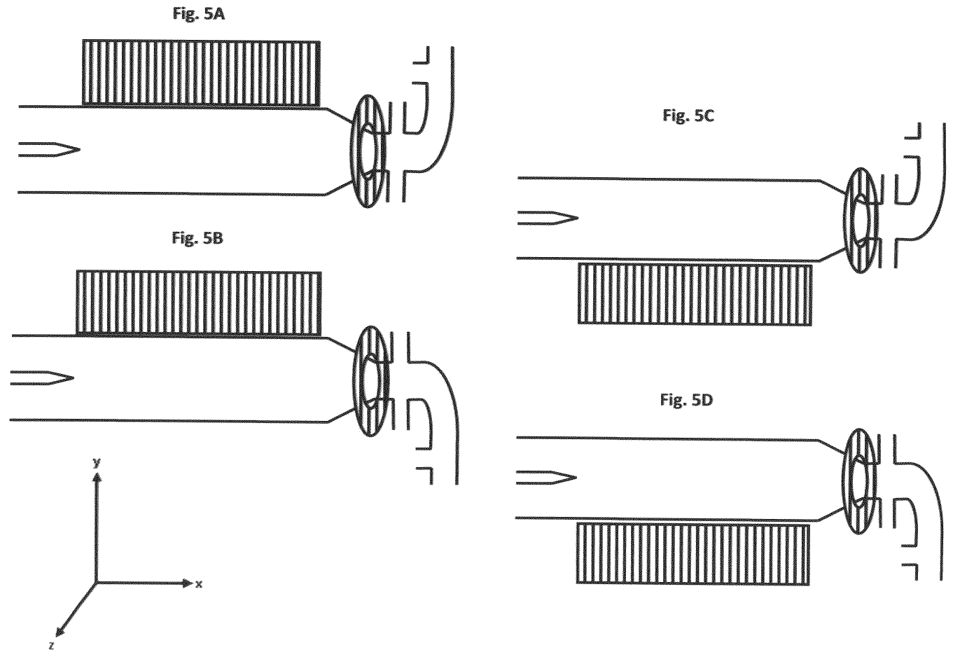

FIG. 4A-D illustrate embodiments of a fluidic concentrating device with a further component (401) that is an annular or toroidal magnet component (ring magnet) surrounding the inlet channel. The ring magnet may comprise one or more magnets positioned in a repelling orientation relative to their magnetic poles, thereby resulting in a symmetric magnetic force along the X-axis of the inlet channel. In one embodiment, the ring magnet comprises a plurality of rectangular magnets configured around the inlet such that the individual repelling fields are aligned within the inlet channel. The force exerting from the ring magnet slows down the particle flow rate by providing a repulsive force to the paramagnetic property of the particles. The ring magnet can be configured in the substantially linear portion of the inlet channel as shown in FIG. 4A with a magnet component substantially aligned along the X-axis of the top of the processing channel and as illustrated in FIG. 4B with a magnet component substantially aligned along the X-axis of the bottom of the processing channel. FIG. 4C illustrates the configuration of FIG. 4A with an additional magnet component or portion (107) extending beyond the processing channel and into the inlet portion. FIG. 4D illustrates the ring magnet configured surrounding an inlet portion of a fluidic concentration device without a magnet component aligned along an axis of the processing channel. Though as illustrated as 401 surrounding the linear portion of the inlet channel in FIG. 4A-D, these embodiments are nonlimiting and the ring magnet may be placed, in other embodiments anywhere along the inlet channel including the nonlinearly aligned portion of the inlet channel.

FIG. 5A-D illustrates embodiments with single magnet components substantially aligned along the top or bottom of the X-axis of the processing channel further comprising ring magnet 401 and nonlinearly aligned inlet channel portions and intersecting channels. One or more of the intersecting channels can also be used as an output to collect particles repelled or otherwise prevented from entering the separation channel.

Many of the embodiments of the particle concentrating device comprise a single magnet component substantially aligned along either the top or bottom of the X-axis of the processing channel. Other embodiments described herein have no magnet component aligned along a substantial portion of the processing channel. In other embodiments, the device may comprise multiple magnet components substantially aligned along top and bottom of the X-axis of the processing channel. Such embodiments provide for magnetic levitation of paramagnetic particles within the channel and thus separation of heterogeneous particles within the processing channel. In accordance with an embodiment, the levitation device includes an upper magnet or a lower magnet that comprises a plurality of magnets that are movably mounted such that the number of magnets that are engaged (i.e., actively creating a magnetic field across the processing section of the fluidic channel) may be controlled, thereby controlling the magnitude and gradient profiles of the magnetic field. Control over the magnetic field as a function of time permits more complex protocols which can be changed at any time during an experiment or assay. Among other advantages over a static system, this permits: more flexible partitioning of samples; higher resolution in the separation of particles; more flexible methods to purge, prime and treat the fluidic paths; and feedback to optimize or change the separation parameters at the time of running an experiment or assay.

In accordance with an embodiment, the upper magnet and the lower magnet comprise elongated rectangular magnets (preferably bar magnets), whose dimensions range from a height (y-axis from (vertical axis)) of about 2 mm to about 25 mm, a width (x-axis from) of about 30 mm to about 80 mm, or to about 95 mm, and a depth (z-axis from) of about 0.5 mm to about 7 mm. Preferably, upper magnet and lower magnet have dimensions ranging from a height (y-axis from) of about 4 mm to about 20 mm, a width (x-axis from) of about 40 mm to about 60 mm, and a depth (z-axis from) of about 1 mm to about 3 mm. The preferred magnet sizes described herein may be achieved by one magnet or by combining multiple magnets. In accordance with an embodiment, depth and the width of upper magnet and lower magnet are substantially the same. In accordance with an embodiment, the height of upper magnet is at least about 25%, 50%, 75%, 100%, 125%, 150%, 175%, 200%, 225%, 250%, 275%, 300%, 325%, 350%, 375%, 400%, 425%, 450%, 475%, or 500% larger than the height of lower magnet. In accordance with an embodiment, the height of upper magnet is about 25% to about 100%, about 100% to about 200%, about 200% to about 300%, about 300% to about 400%, about 400% to about 500%, or about 500% to about 600% larger than the height of lower magnet. In accordance with an embodiment, the height of lower magnet is at least about 25%, 50%, 75%, 100%, 125%, 150%, 175%, 200%, 225%, 250%, 275%, 300%, 325%, 350%, 375%, 400%, 425%, 450%, 475%, or 500% larger than the height of upper magnet. In accordance with an embodiment, the height of lower magnet is about 25% to about 100%, about 100% to about 200%, about 200% to about 300%, about 300% to about 400%, about 400% to about 500%, or about 500% to about 600% larger than the height of upper magnet.

In accordance with an embodiment, the distance between the upper and lower magnets and the fluidic channel, capillary or central processing section along the vertical axis is at least about 1 micron, 10 microns, 50 microns, or 100 microns and/or is no greater than about 500 microns, 1 mm, 2 mm, 3 mm, 4 mm, or 5 mm. In accordance with an embodiment, the distance between either of the magnets and the fluidic processing channel is between about 1 micron to about 5 mm along the vertical axis, and preferably about 10 microns to about 2 mm.

In accordance with an embodiment, the vertical distance between the upper magnet and the fluidic processing channel is at least about 25%, 50%, 75%, 100%, 125%. 150%, 175%, 200%, 225%, 250%, 275%, 300%, 325%, 350%, 375%, 400%, 425%, 450%, 475%, or 500% greater than the vertical distance between lower magnet and the fluidic processing channel. In accordance with an embodiment, the vertical distance between the upper magnet and the fluidic processing channel is at least about 25% to about 100%, about 100% to about 200%, about 200% to about 300%, about 300% to about 400%, about 400% to about 500%, or about 500% to about 600% greater than the vertical distance between the lower magnet and the fluidic processing channel.

In accordance with an embodiment, the vertical distance between the lower magnet and the fluidic processing channel is at least about 25%, 50%, 75%, 100%, 125%, 150%, 175%, 200%, 225%, 250%, 275%, 300%, 325%, 350%, 375%, 400%, 425%, 450%, 475%, or 500% greater than the vertical distance between upper magnet and the fluidic processing channel. In accordance with an embodiment, the vertical distance between the lower magnet and the fluidic processing channel is at least about 25% to about 100%, about 100% to about 200%, about 200% to about 300%, about 300% to about 400%, about 400% to about 500%, or about 500% to about 600% greater than the vertical distance between the upper magnet and the fluidic processing channel.

In accordance with an embodiment, upper magnet and lower magnet are permanent magnets or electromagnets. In accordance with an embodiment, the maximum energy product of upper magnet and lower magnet ranges from about 1 Mega-Gauss Oersted to about 1000 Mega-Gauss Oersted, and more preferably ranges from about 10 Mega-Gauss Oersted to about 100 Mega-Gauss Oersted. In accordance with an embodiment, the surface field strength of upper and lower magnets ranges from about 0.1 Tesla to about 100 Tesla, and more preferably ranges from about 1 Tesla to about 10 Tesla. In accordance with an embodiment, the remanence of upper and lower magnets ranges from about 0.5 Tesla to about 5 Tesla, and more preferably ranges from about 1 Tesla to about 3 Tesla.

In accordance with a preferred embodiment, an asymmetric magnetic field is achieved by using a stronger magnetic material on one side of a processing channel and a weaker magnetic material on the opposite side of the processing channel. In accordance with a preferred embodiment, an asymmetric magnetic field is achieved by using a magnetic material on one side of a fluidic channel and a substantially similar magnetic material on the opposite side of the fluidic channel. In accordance with such embodiment, upper magnet and lower magnet may be substantially the same size. In accordance with such embodiment, upper magnet may comprise an alloy of neodymium and iron, lower magnet may comprise samarium-cobalt, and wherein both magnets are substantially the same size. Alternatively, upper magnet may comprise samarium-cobalt, lower magnet may comprise neodymium, and wherein both magnets are substantially the same size.

In accordance with an embodiment, alternative magnet configurations may be used. Referring to, the device in accordance with the present invention may include multiple upper magnets and multiple lower magnet positioned around a fluidic channel. Upper magnets may include an anterior upper magnet, a central upper magnet, and a posterior upper magnet. Lower magnets may include an anterior lower magnet, a central lower magnet, and a posterior lower magnet.

In accordance with another magnet configuration, the device may include an anterior upper magnet, a posterior upper magnet, an anterior lower magnet, and a posterior lower magnet, wherein the magnets are positioned around fluidic channel. The anterior upper magnet and the posterior lower magnet are positioned in a magnetic repelling orientation. Descriptions of asymmetrical magnetic levitation devices are further described in International Patent Application PCT/US19/24138 which is incorporated herein by reference.

FIG. 7A-D illustrate embodiments of the dual magnet component particle concentrating device with optional ring magnet 401 and optional inlet channel portions, 201, 202, and 203 in exemplary orientations.

Certain embodiments of the particle concentrating device described herein have no magnet component aligned along a substantial portion of the processing channel.

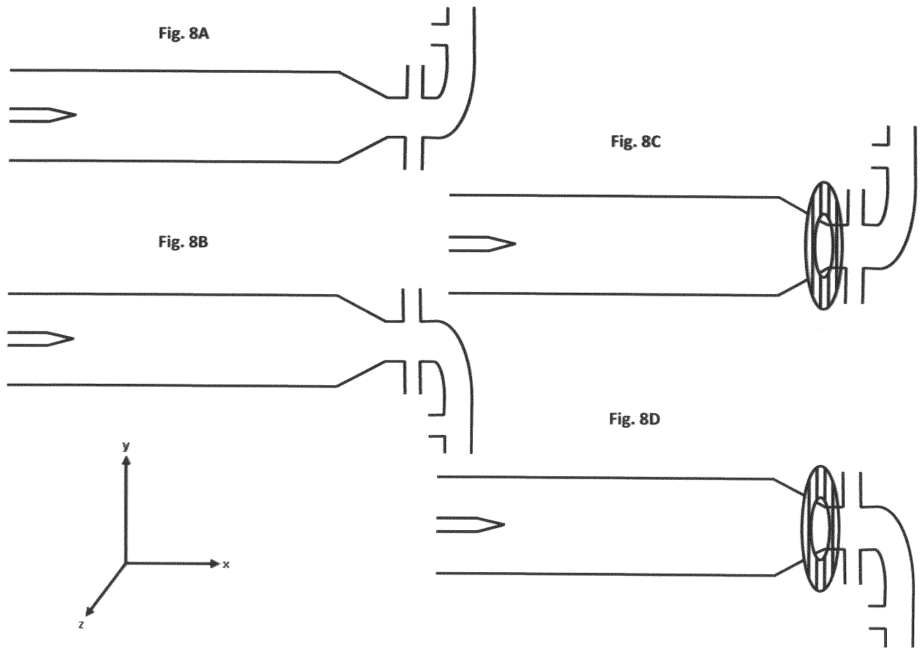

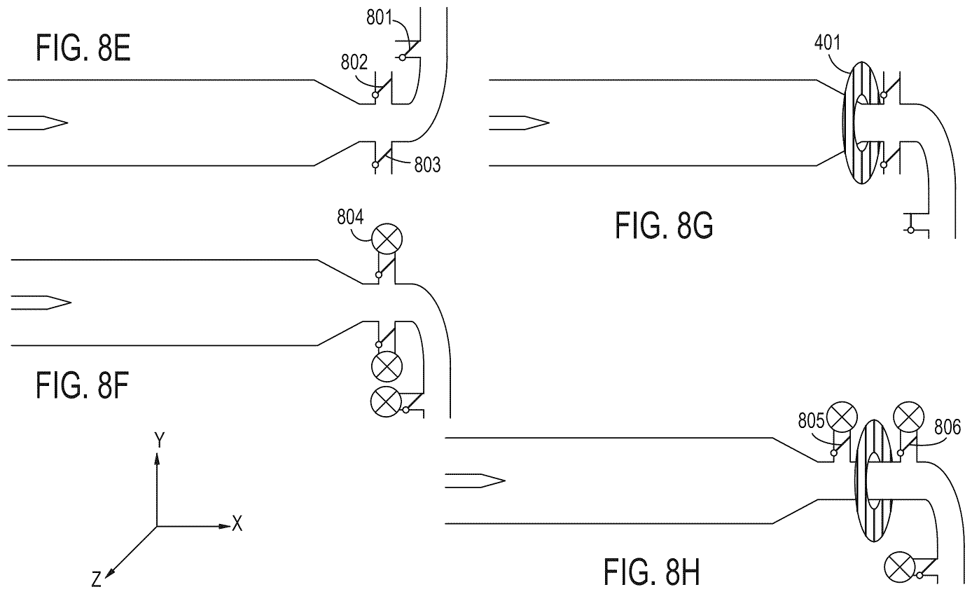

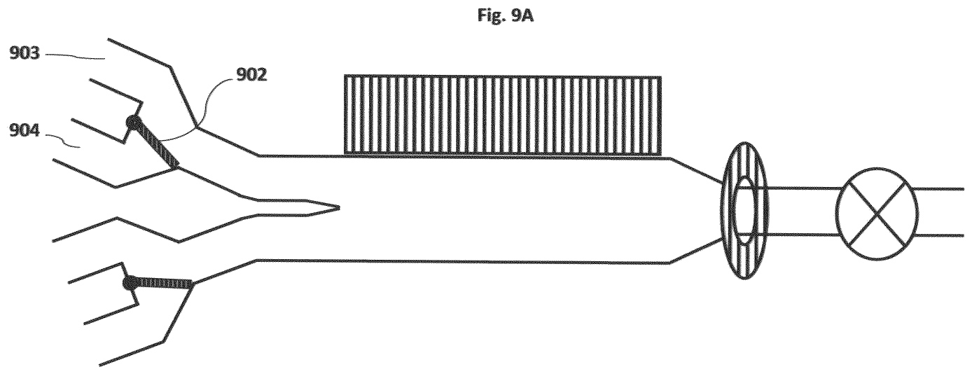

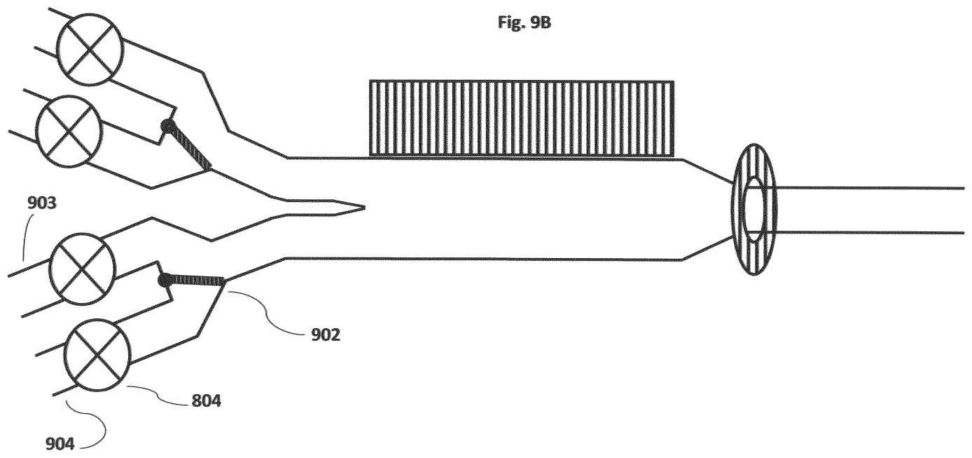

Particle concentration is achieved through a combination of sedimentation and optionally preconcentration of particles in the inlet channel through surmountable magnetic repulsion of particles within the inlet channel and through flow-mediated preconcentration that may optionally be enhanced through inlet channel geometry. FIG. 8A-D illustrates embodiments of device configurations. FIG. 8E-H illustrates the incorporation of inlet channel valves (801, 802, 803) controlling movement of fluid to optional intersecting channel(s). A pump (804) may be associated with any channel, inlet, inlet intersecting, outlet, diverting, etc. The pump, as further described herein, may be configured in various embodiments to provide positive or negative pressure as required to drive fluids in the corresponding channel and cumulative flow when operated in multiple pumps associated with channels in fluidic communication. FIG. 8H illustrates a particle concentrating device with inlet intersecting channels 805 and 806. As shown in FIG. 8H, an inlet channel intersecting channel may be upstream, downstream, or both of the ring magnet. In an embodiment, an intersecting channel (806) is provided only in the portion of the inlet channel that is substantially linear to the processing channel that is upstream of the ring magnet.

FIG. 9 illustrates an embodiment of a single magnet component particle concentration device. FIG. 9A illustrates particle concentrator device with a pump (804) driving flow from the inlet channel into the processing channel. FIG. 9B illustrates a particle concentration device with a plurality of pumps (804) individually controlling flow rate of outlet channels. The combined flow rates are additive for the flow in the processing and inlet channel. Flowrates can range from 0.1 uL to 1 mL per minute depending on sample fluid characteristics such as viscosity and particle concentration and inlet channel, processing channel, and outlet channel dimensions and configuration. In a preferred embodiment, processing channel and outlet channel flowrates may be from about 0.2 uL per minute to about 200 μL per minute, or from about 0.5 uL per minute to about 40 uL per minute. Concentration can also be performed under stop-flow or intermittent-flow operation. Valve (902) can be actuated divert the outlet channel into an outlet port (903) or (904). In some embodiments, inlet channel pump(s) and outlet channel pump(s) are employed in either a positive or negative pressure configuration, respectively in relation to the to the processing channel. Under stop-flow conditions, the separation of the sample is performed and the sample is removed into separate outlet channels by resuming flow within the processing channel.

In accordance with the present invention, the device may include one or more pumps to drive fluid through the device. “Pump” is used to refer to any device which applies a difference in pressure between different locations in the device. Pumps may be placed on either the inlet side of the system (pushing fluid toward the outlet(s)), or on the outlets (pulling liquid from the inlet(s)), or a combination of both. The difference in pressure may be positive or negative. The pressure difference may be applied in common across multiple outlets or inlets, or may by arranged such that each outlet or inlet has a directly-applied pressure difference. Pressure differentials may be applied, in some embodiments, to overcome particle preconcentration forces, magnetic, sedimentary, or a combination of forces in the inlet channel. The pumps may be variable to allow control of the applied pressure difference. Pump types include, but are not limited to: positive displacement pumps such as syringe pumps; peristaltic pumps; diaphragm pumps; regulated static pressure sources; gravitationally-controlled pressure sources such as elevated or lowered volumes of liquid; and manual sources of pressure such as plastic or foil blisters.

In some embodiments, a pump may be included on inlet line(s) to generally drive fluid through the channel structure, and also included on certain outlet channels or ports to drive fluid through directed outlet lines. For example, pumps may be included on one or more outlet lines associated with the particle enriched or particle depleted layer height or heights of one or more particles of interest. In addition, all outlet lines may include a variable pump that may be activated or deactivated based on the anticipated flow stream height or heights of one or more particles of interest. Similarly, external pumps may be controlled to provide a variable pressure differential. Embodiments may further comprise additional components including: a receptacle for holding one or more outlet collection tubes; a receptacle for holding one or more input tubes; a component comprising a receptacle for one or more tubes which is temperature-controlled, for example a cold plate which stores one or more outlet tubes at a temperature close to 4° Centigrade; or a microplate holder, which may include positioning means to couple inlets or outlets to wells in the microplate. In an embodiment, an outlet channel or port is fluidically coupled to a pipetting robot. The pipetting robot may be integrated with the particle concentration device to selectively dispense aliquots or fractions of a concentrated particle population from the processing channel and alternatively, or additionally, dispense aliquots or fractions of a particle depleted effluent from the processing channel. The device may also be integrated with a microprocessor or computer that is programed to record, analyze, and/or control the fluid and/or particle flow and separation through the device.

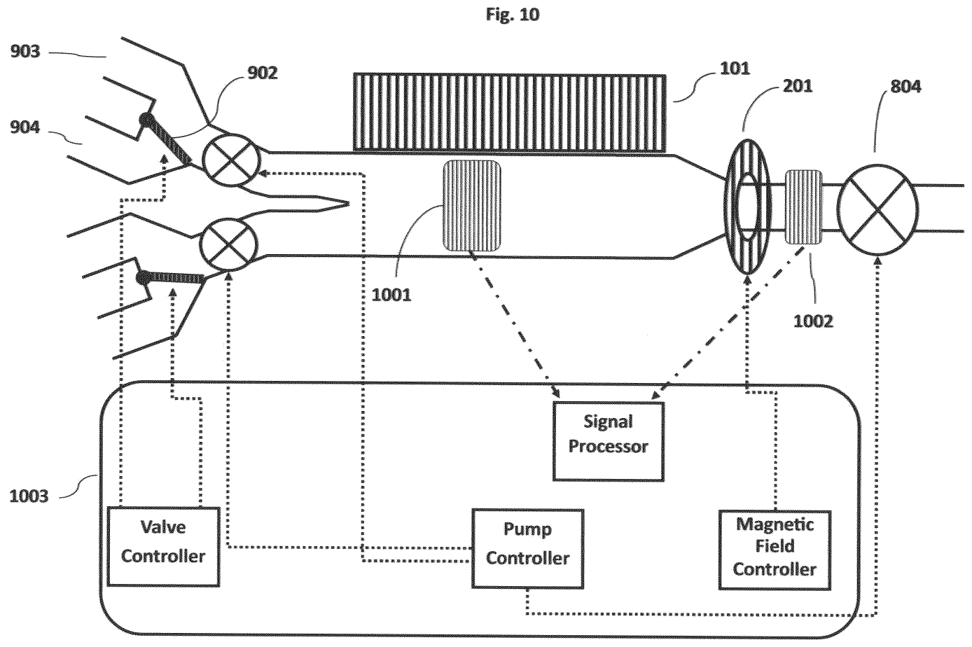

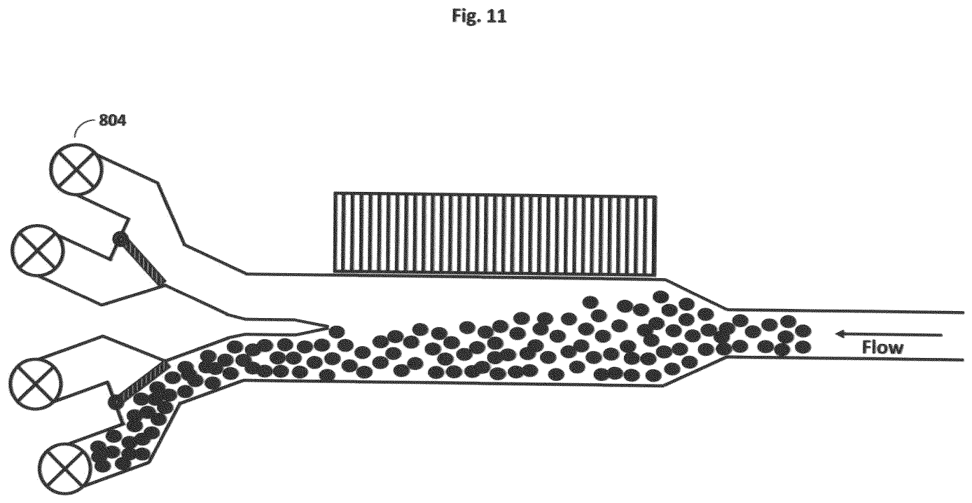

FIG. 10 illustrates a particle concentration system comprising an exemplary particle concentration device. The device may further comprise one or more sensors. For example, a processing channel sensor (1001) and inlet channel sensor (1002) can be implemented into the system. A system integrator (1003) can comprise a signal processor to receive signals from sensors (1001) and (1002) and through preprogrammed or programmable commands, control other components such as inlet pump(s) (804) or outlet channel pumps, magnetic field of ring magnet (201) or other electromagnetic components, valves or other interfacing devices or components. A sensor is selected from an optical sensor, capacitive sensor, conductive sensor, thermal sensor, flowrate sensor, ultrasonic sensor, gravimetric sensor, magnetic field sensor, or combinations thereof. In embodiments, the sensor is a photodetector, a multipixel imaging detector, a magnetic field detector, an electrochemical detector, an optical phase detector, a scatter detector, a Hall sensor, a magnetoresistive sensor, a bolometric sensor, surface acoustic wave sensor, a biosensor, or combinations thereof. Sensors may be further comprise one or more sensors inside, or adjacent to, portions of the processing channel or inlet channel to detect presence or absence or quantity of particles or other physical or chemical properties of the particles or sample flow stream.

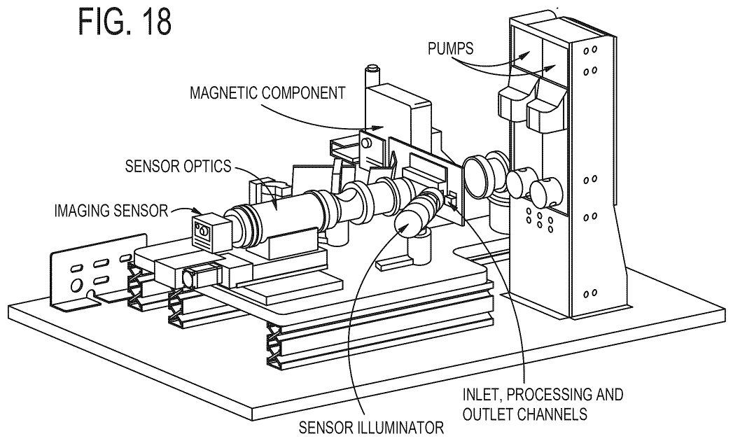

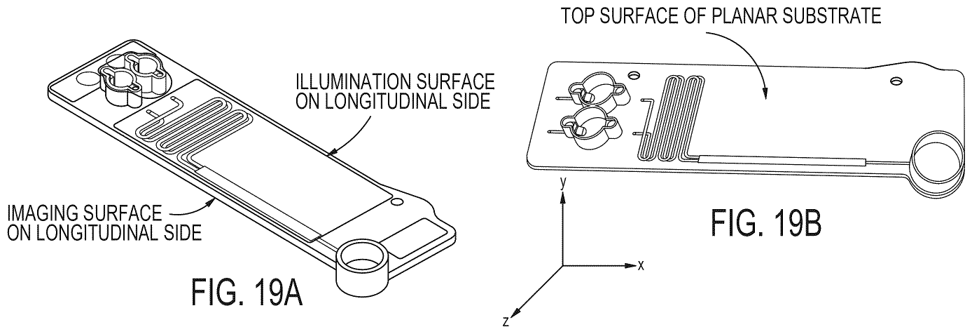

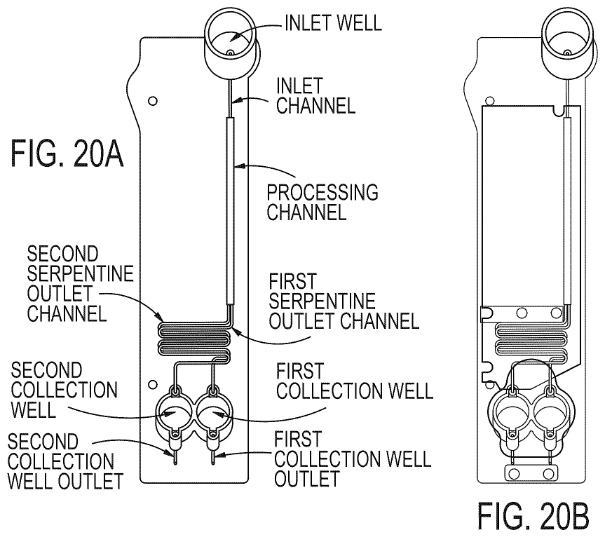

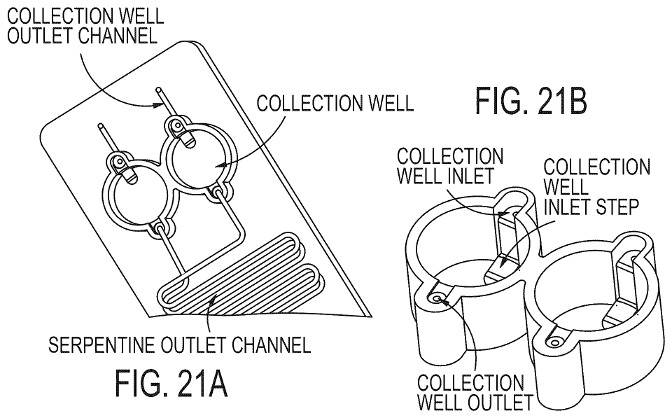

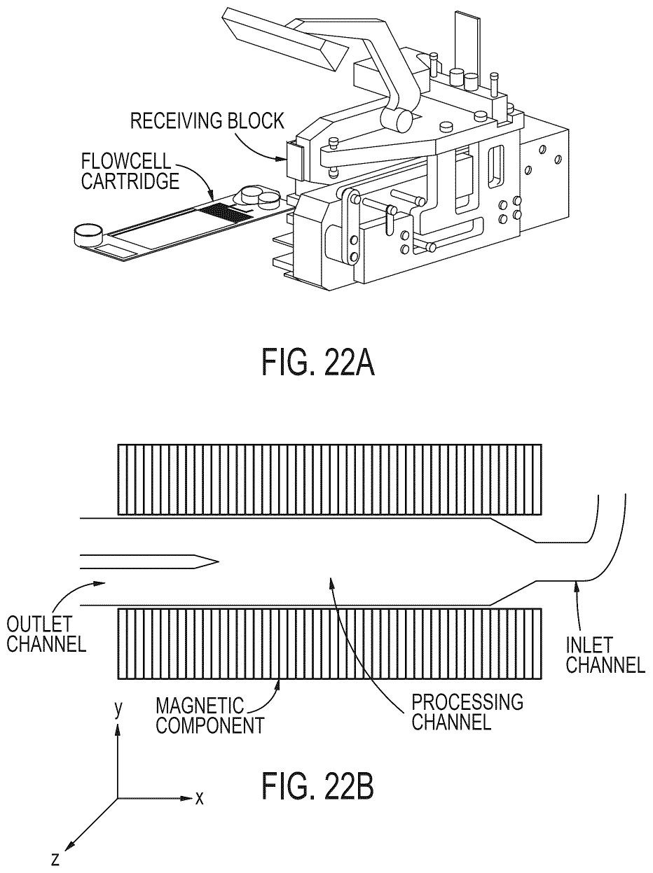

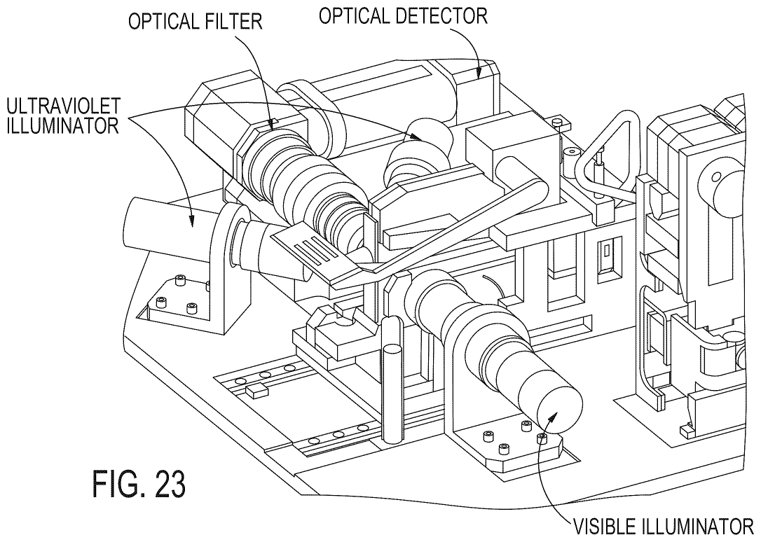

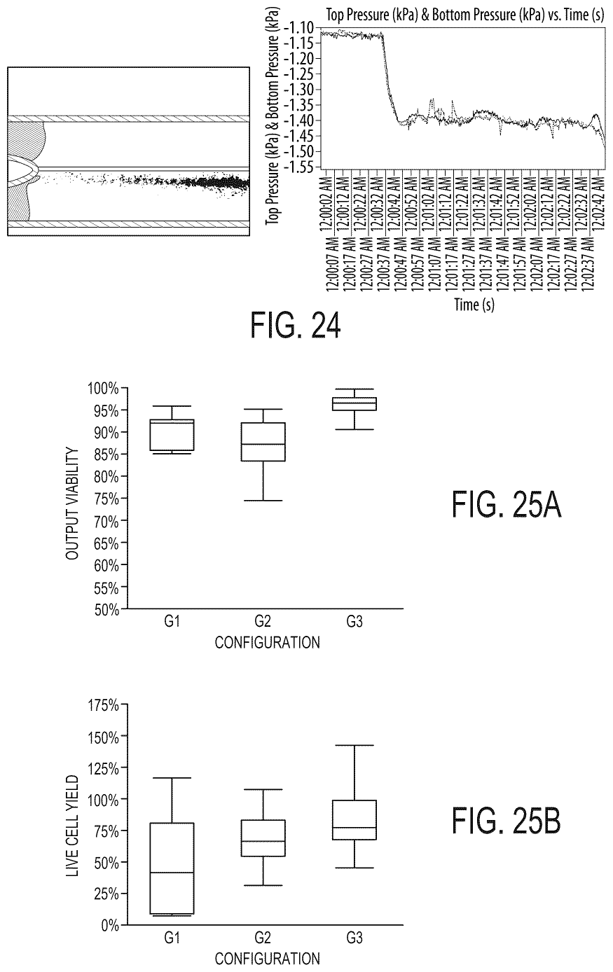

As illustrated in the plan view of particle concentrating device in FIG. 18, the device may further comprises a visualization component or imaging sensor, a sensor illuminator, and sensor optics. Visualization component may comprise any device which enables or enhances the ability to view in real time and/or to record particles as they pass through processing channel, thereby enabling observation and/or measurement of the isolation of the particles, including the extent of particle isolation and/or the rate of particle isolation. Visualization may also include analysis of the size, shape, or other characteristics of the particles and/or other components of the sample. In accordance with an embodiment, the material used to surround and thereby define the processing channel is clear or transparent along at least a segment of the processing channel to facilitate observation of particles passing therethrough. The visualization system may employ optics to allow bright-field illumination, dark-field illumination, and/or fluorescent detection of sample components.

In an embodiment, the device comprises two optically clear or transparent channel segments, with each on opposite sides of channel. In accordance with this embodiment, the visualization component is positioned on one side and focused through one of said clear or transparent segments, and an illumination component positioned on the opposite side and focused through the second of said clear or transparent segments. The illumination component is configured to provide sufficient light to facilitate the visualization of the particles within processing channel by the visualization component. In another embodiment, the device includes one clear or transparent segment, on one side of channel. In accordance with this embodiment, the visualization component is positioned on one side and focused through said clear or transparent segment, and an illumination component positioned on the same side and focused through said clear or transparent segments. The illumination component is configured to provide sufficient light to facilitate the visualization of the particles within processing channel by the visualization component.

In a preferred embodiment the illumination system is a source of visible or ultraviolet light. The illumination source can be configured to illuminate a sample in an optically transparent flow channel, including a processing channel, wherein the light is transmitted through the flow channel to an optical sensor opposite the illumination source. In an embodiment, the illumination source is angularly adjacent to the optical sensor so that light from the optical source is reflected from the sample in the flow channel into the optical sensor. In an embodiment, the optical source is a source of ultraviolet light and is constructed and arranged to illuminate the sample within a flow channel such that visible light from natural florescence of artificial florescence associated with the sample is detected by the optical sensor.

### V. Particle Isolation and Concentration Methods

Organic or inorganic particles, may be concentrated by the methods of the present invention. The particles may be biological entities such as cells, cell fragments, organelles (e.g., cell nuclei), clusters, tissue, tissue components, microorganisms including bacteria, fungi (yeasts and molds), viruses, protozoa, and algae and fragments, organelles, clusters, and other components thereof. Particles can be macromolecules, complexes, chelates, conjugates, crystals, amorphous solids, gels, coagulates, and the like. DNA, RNA, proteins, are concentratable under methods of the present invention. Beads, shells, nanoparticles, laminates, and precipitates and coprecipitates may likewise be concentrated. Numerous applications require the isolation of particles, including applications requiring the separation of like particles from other particles, identification of particles, and the treatment or otherwise manipulation of particles. Such applications include, but are not limited to, separating live and dead cells, separating cell nuclei from live and dead cells and nuclear debris, isolation and/or treatment of circulating tumor cells, emulsion PCR enrichment, production of plasma such as platelet rich plasma, isolating sperm for specific traits such as gender selection, bacterial load testing, antibiotic resistance testing, identification of sepsis or blood contamination, immune cell isolation, compound screening, exosome separation, or extracellular vesicles separation. The particle isolation methods of the present invention may be utilized in any of these applications.

Particles present in a sample medium are concentrated in a particle concentrating device under conditions that substantially enrich particle concentration and substantially deplete a layer of sample medium. Sample medium with heterogeneous particle populations may be selectively enriched based on size, density, and paramagnetic heterogeneity and selective orientation of magnetic forces and processing channel flow rates. The heterogeneous population of particles may be derived from biological samples. In some cases, the biological samples are, as illustrating examples, a bodily fluid including blood, saliva, urine, sperm, plasma, serum, and stool; swabs including skin, anal, nasal and vaginal swabs or environmental swabs from a door handle; and proximal fluids including tears, lavage fluid from lungs, or interstitial tissue fluids from a breast. In some cases, the biological samples are, as illustrating examples, live and dead cells, lysed cells, circulating tumor cells, nucleic acids, nucleotides, amino acids, peptides, proteins, antigens, antibodies, or immune cells (e.g., white blood cells, T cells, phagocytic cells). In some cases, the biological samples are, as illustrating examples, a biomolecule, cell, protein, lipid, carbohydrate, microorganism, virus, viron, or bacteria.

Level of concentration of the particle enriched fraction over particle concentration in the sample medium is at least 30%, preferably 40%, 50%, 60%, 70%, 80%, 90%, 95%, or 99%. In an embodiment, the particle depleted fraction is substantially free of particles.

Prior to introduction into the processing channel, reagent fluids or gases may be introduced into the flow stream prior to, concurrently with, or subsequent to the passage of a sample medium along a position containing an intersecting channel. In one embodiment, a paramagnetic medium, buffer, flocculating agent, sample preconditioning reagent or reactant may be introduced and mixed into the sample medium a preparation for concentration or isolation or analysis in the processing channel.

Particles are introduced into a processing channel at a flow rate and subjected to sedimentation and/or magnetic repulsion and/or attraction within the processing channel to form particle enriched and particle depleted layers within the flow stream. In various embodiments, the formation of particle enriched and particle depleted layers within the flow stream occur under continuous flow, stop flow, or intermittent flow conditions. Collection of the particle enriched and/or particle depleted layers is performed as the layers are split in the processing channel flow stream. Flowing output from the processing channel, the particle enriched streams and/or the particle depleted streams may be selectively channeled for aliquoting or fractionation. Once the particles of interest reach their equilibrium (or near equilibrium) height in a flow layer within the processing channel, they pass through a splitter that divides the processing solution into multiple fractions. Because the particles of interest are geometrically isolated within the processing solution, substantially all the particle of interest are retained within the effluent of certain geometric fractions. The geometric effluent fraction or fractions containing the particles of interest are then collected and may be recombined if the particles of interest are present more than one fraction, thereby isolating the particles of interest. In some embodiments it may be necessary to separate the cells from the paramagnetic medium. This may be done through dilution if separation of the cells from the paramagnetic medium is desired.

Alternatively, the division of sample solution into each effluent fraction may be achieved by increasing or decreasing the fluid flow toward individual outlets, such that the ratio of division can be modified. In accordance with an embodiment the ratio may be modified by up to 50%. For example, if the splitter comprises two channels with equal cross-section, the geometric ratio of division is 1:1. By withdrawing a larger (or smaller) amount of fluid into one fraction through the application of a larger (or smaller) pumping rate than is applied to the other fraction, the ratio of division can be altered, e.g. to about 3:1, 4:1, 5:1, 6:1, 7:1, 8:1, 9:1 or 10:1. In a preferred embodiment, the division for such geometric ratio would be within the range of about 2:1 to about 1:2. In a further preferred embodiment, the division for such geometric ratio would be within the range of about 10:1 to about 1:10. The division could be about 1:10, about 1:9.5, about 1:9, about 1:8.5, about 1:8, about 1:7.5, about 1:7, about 1:6.5, about 1:6, about 1:5.5, about 1:5, about 1:4.5, about 1:4, about 1:3.5, about 1:3, about 1:2.5, about 1:2, about 1:1.5, about 1:1, about 1.5:1, about 2:1, about 2.5:1, about 3:1, about 3.5:1, about 4:1, about 4.5:1, about 5:1, about 5:5, about 5.5:1, about 6:1, about 6.5:1, about 7:1, about 7.5:1, about 8:1, about 8.5:1, about 9:1, about 9.5:1, about 10:1 each ratio respective of an upper outlet channel flowrate to a lower channel flowrate.

In one embodiment, the sample fluid comprising concentrated particles passes through the fluidic channel at a relatively slow rate until it reaches the tailing end of the processing segment, where the flow rate increases at least 2 times. In a preferred embodiment, the flow rate of the sample fluid increases by at least 4 times at the tailing end of the processing segment. In some cases, the sample fluid is exposed to a magnetic field created by the magnet ring or the pair of upper and lower magnets as described herein. In some cases, the sample fluid is not exposed to a magnet field.

The aliquots or fractions may be diverted into collecting chambers or containers, such as tubes or plates. The fractions or aliquots may be subjected to further processing, analysis, or reaction. In an embodiment, the collecting chambers, plates, wells, and/or tubes comprise a pre-determined quantity of a material required or a subsequent processing step, allowing the user to not only concentrate the cells but also to transfer them from one medium to another, or to add a reagent. Exemplary reagents, include but are not limited to, reagents for RNA isolation, DNA isolation, mRNA isolation, protein isolation, growth media, culture media, and fixative. Isolated aliquots and/or fractions may be subjected to further processing comprising magnetic levitation and separation, chemical or biochemical analysis, fractionation, derivatization, sequencing sample preparation, mass-spectral analysis, NMR analysis, microscopic analysis, FACS sorting and analysis, and X-ray diffraction analysis. Biological cells may be collected and used in diagnostic or therapeutic procedures in their natural collected state or subjected to genetic or biochemical modification.

During residence in an inlet, processing, or outlet channel, particles may be interrogated for a property including speed, density, biological, chemical, genetic, taxonomy, configuration, viability, concentration, or orientation. The interrogation may be performed with one or more sensors or array of sensors within, adjacent to, or interrogatively linked to an inlet, processing, outlet channel, or combinations thereof, and collection chambers, wells, plates, or tubes. The detected characteristics can be used for independent analysis and can be utilized by a system controller component to control or automate system operation. System components and parameters under control include sample medium flow rate, magnetic field strength, valve actuation for diversion, collection, fractionation, and subsequent reaction conditions. Sensors within the inlet channel are integrated in an embodiment to be operatively linked to the system controller to provide for the automated introduction of reagent fluid or gas into the sample stream in the inlet channel.

FIG. 11 illustrates the concentration of particles utilizing a device of the present invention. Particles are introduced into the processing channel via flow of the sample medium through the inlet channel. Sedimentation and/or magnetic repulsive forces concentrate particles in the lower layer of the flow stream and the concentrated particles are collected through the lower outlet channel. The particle depleted layer is collected via the upper outlet channel. This method comprises the steps (i) providing a low volume fluidic device with a processing channel, an inlet channel, and a plurality of outlet channels, (ii) flowing a particle containing sample through the inlet channel into the processing channel under conditions to produce a sample flow stream with at least a particle enriched layer and a particle depleted layer, (iii) flowing the particle enriched layer through a first outlet channel to produce a particle enriched flow stream, (iv) flowing the particle depleted layer through a second outlet channel to produce a particle depleted flow stream, and (v) collecting one or more of the flow streams from one or more of the outlet channels.

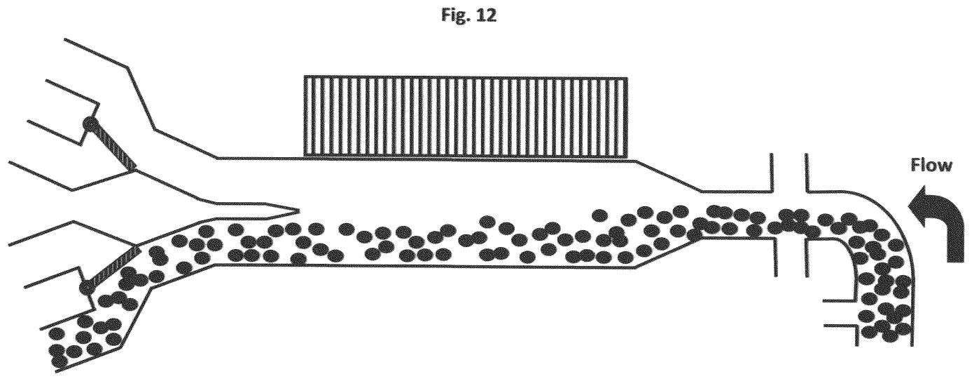

FIG. 12 illustrates a flow assisted particle concentration method wherein the inlet channel the inlet channel is further comprised of portion substantially linear to the processing channel and a portion that is nonlinearly aligned to the processing channel connecting at a portion that is angled with an angle, theta (Θ). The particle containing sample medium is flowed through the inlet channel at a flowrate sufficient to allow sedimentation within the nonlinearly aligned portion of the inlet channel to concentrate the particles within the inlet channel. Flow of the particle containing sample into the processing channel is maintained to provide for continued or further sedimentation optionally with magnetic repulsion of the particles to form a particle enriched layer and a particle depleted layer of the flow stream. The respective layers of the flow stream are collected at their respective outlet channels. Without being bound to any theory, concentration of particles is presumably due to a combination of the tendency of particles with a density greater than the liquid they are suspended in (e.g., water, solvent) to settle in the presence of gravity, as well as repulsion from the magnetic fields. The particles may be allowed to sediment for at least 1 min, at least 2 min, at least 5 min, at least 10 min, at least 20 min, at least 30 min, or at least 60 min. The particles may be allowed to sediment for at least 10 seconds, at least 20 seconds, at least 30 seconds, at least 40 seconds, at least 50 seconds, at least 60 seconds, at least 100 seconds, or at least 200 seconds. In some embodiments, the concentrated particles pass through the fluidic channel at a flow rate of at most 50 ul/min (microliters per minute), at most 40 ul/min, at most 30 ul/min, at most 20 ul/min, at most 10 ul/min, at most 5 ul/min or at most 2 ul/min. In some embodiments, the concentrated particles pass through the fluidic channel at a flow rate of about 20 ul/min, which will bring the liquid passing through the flow device but allow particles to accumulate outside the magnetic field. In some embodiments, the concentrated particles pass through the fluidic channel at a flow rate of about 10 ul/min, which will bring the liquid passing through the flow device but allow particles to accumulate outside the magnetic field. The liquid passing through the flow cells would be deposited through one of the outlet channels, which lead to the collecting chambers. Once the majority of the liquid has passed through the device and particles are seen to enter the flow cell, the liquid with suspended particles (e.g., suspended cells) would be passed at a higher flow rate into the second output channel, which leads to collecting chambers specific for collecting particles of interest.

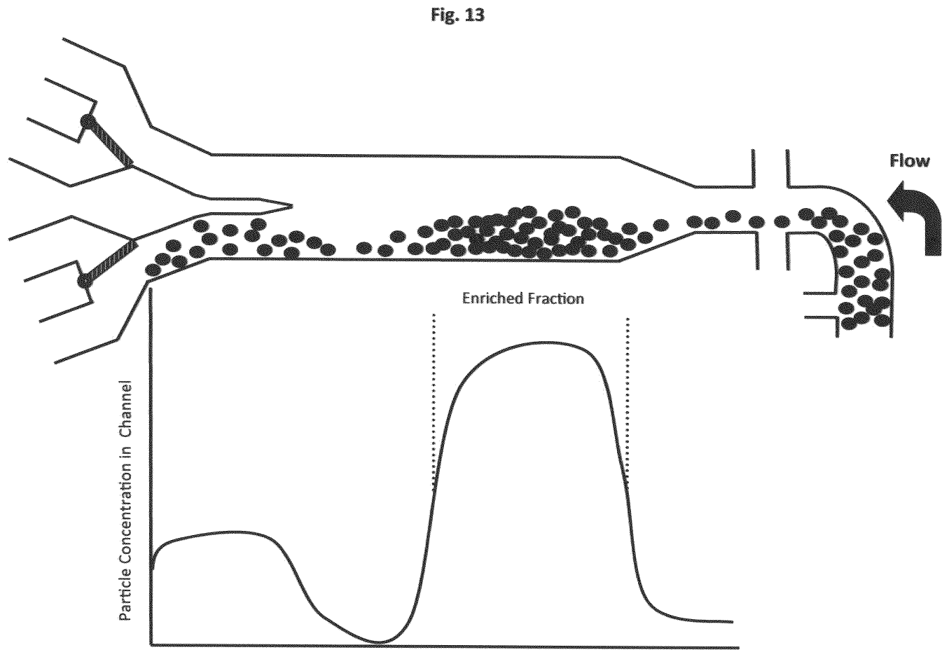

FIG. 13 illustrates the flow assisted formation of particle concentration fractions within the processing channel. Concentrated (particle enriched) fractions are collected via the lower outlet channel to provide highly enriched aliquots of the particles. The particle enriched portions of the flow stream may be identified by sensors such as imaging sensors observing particles within the processing channel, or temporally via timed collection of fractions for samples where fractionation patterns within the processing channels are characterized.

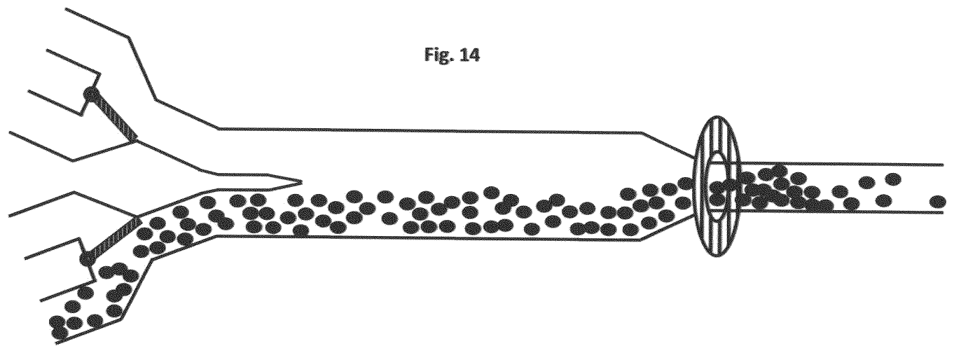

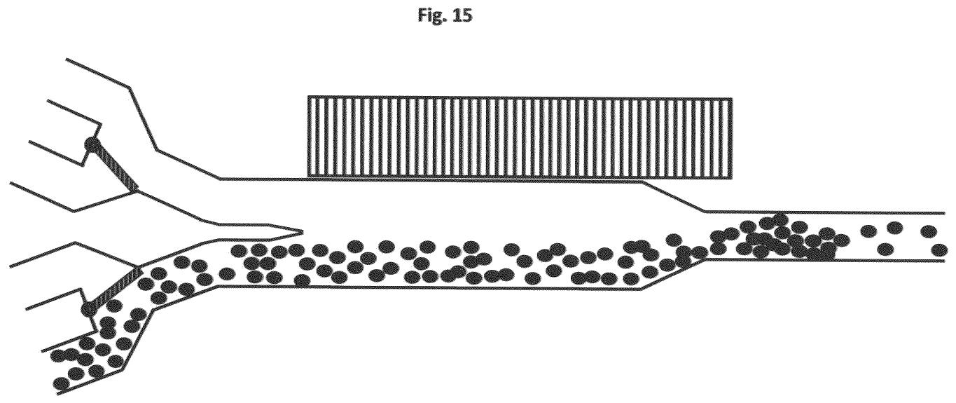

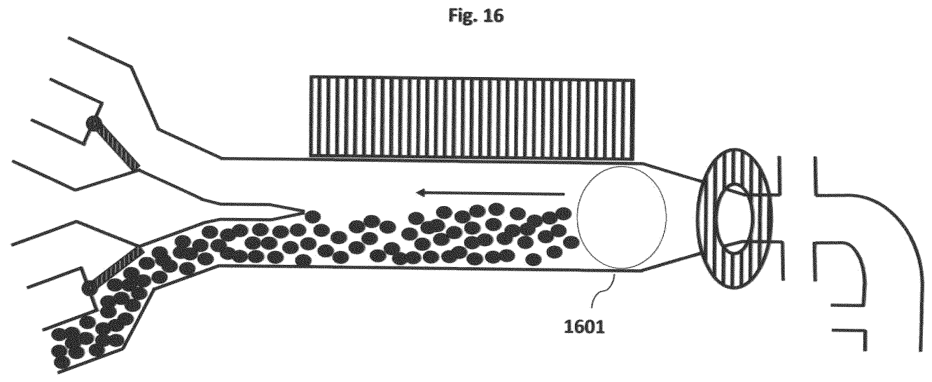

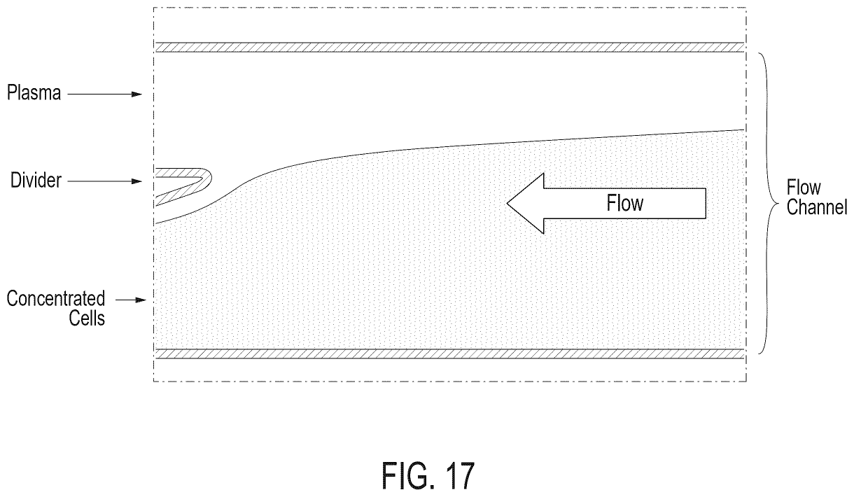

FIG. 14 illustrates a preconcentration of particles within the inlet channel by applying a magnetic repulsive force to the particles prior to entry into the processing channel. Entry of the particles into the processing channel is accomplished by discontinuing or reducing the magnetic repulsive force within the channel or by modulating the flow stream to overcome the magnetic repulsive force or a combination of magnetic fore reduction and flow stream modulation. Modulation of the flow stream can include an increase in the flowrate of the sample medium flow stream, or fluctuating the flow stream such as by performing a pulsatile inlet channel pressure to provide for introduction of the particles into the processing channel.

FIG. 15 illustrates a method of particle concentration wherein a magnetic component aligned along the X-axis of the processing channel is provided to extend a magnetic repulsive force within the inlet channel to inhibit entry of particles into the processing channel. Flowrate is maintained to provide concentration of particles in the inlet channel. Upon sufficient particle preconcentration, magnetic repulsive forces are overcome and particles are introduced into the processing channel. The degree of preconcentration and is modulated by the flow rate of sample medium. Particles entering the processing channel are sedimented/magnetically repulsed or attracted into the particle enriched layer of the processing channel flow stream and collected in an outlet channel.

In an embodiment of the methods according to FIG. 16, gas bubble or immiscible droplet (1601) is introduced following entry of the sample medium into the processing channel. The bubble or droplet is flowed posterior to the sample medium in the processing channel to substantially flush remaining particle enriched layer and particle depleted layer from the processing channel into the outlet channels. This allows for substantially complete isolation of sample fractions and provides for processing of discrete samples or fractions of samples. The frequency of introduction of air can be adjusted to break a large initial sample volume into smaller units for more efficient processing.

An embodiment of the methods of the present invention is a method of fractionating a blood sample comprising providing a whole blood sample or diluted blood sample, and subjecting the sample to a sample concentration method as described herein and isolating plasma and/or blood cells from a whole or diluted blood sample. The blood sample may be a whole or diluted sample from a peripheral blood sample, umbilical cord blood sample, fetal blood sample, or arterial blood sample with a volume of from about 50 uL to about 50 mL, 50 uL to about 20 mL, 50 uL to about 10 mL, or about 50 uL to about 5 mL. In accordance with the method, the separated plasma fraction may contain less than about 1% to less than about 0.01% of the blood cells in the blood sample or be substantially free of blood cells. The isolated blood sample fractions may be used in a diagnostic assay such as an enzyme immunoassay, chemiluminescent immunoassay, hemagglutination/particle agglutination assay, nucleic acid amplification technology assay, a drug assay, a forensic assay, or a genetic trait assay.

As an example of blood cell concentration/plasma separation, a cord blood sample was fractionated into plasma and cellular fractions in a particle concentrating device and method of the present invention. A (10 mL) blood sample was obtained and paramagnetic medium of gadobutrol to obtain a final concentration of 100 nM was added. A total sample volume of 500 microliters was introduced through the inlet channel of a processing channel configured with single magnet component substantially aligned along the X-axis of the top of the processing channel (dimensions X×Y×Z 50×1.9×1 mm), allowed approximately 5 minutes for equilibration, and then flowed at a starting flow rate of 20 microliters per minute. The combined flow rate of 20 microliters per minute comprised 10 microliters per minute flowing to the bottom collection channel and 10 microliters per minute flowing to the top collection channel. After monitoring separation using live imaging, the flow rate was increased to 50 microliters per minute and then to 100 microliters per minute. The ratio of flow rates into the top collection channel and bottom collection channel respectively was adjusted from 1:1 to 4:1 and then an asymmetry in excess of 10:1 (with 10 being to the bottom collection channel, and 1 being to the top collection channel) to draw a higher proportion of the fluid into the bottom channel, and thereby maintain high purity. Concentration of the blood cell enriched layer occurred into the bottom layer of the flow stream and collection was performed through the bottom outlet channel. FIG. 16 is a micrograph (scale: the flow channel height is 1.9 mm) of the processing channel at the anterior region and the sample splitter showing substantial concentration and isolation of the lower blood cell layer and upper plasma layer.

### VI. Flowcell Cartridge

Precision, accuracy, and reproducibility are requirements scientific apparatus. Additional considerations include ease of use and manufacturability. The flowcell of the present invention has all of the required hallmarks of usable device which enable scientific experimentation and development that has been either impossible or sometimes only achievable by lengthy and/or complex and costly procedures.

A flowcell cartridge of the present invention comprises a planar substrate comprising an upper surface and a lower surface, a first longitudinal side forming an imaging surface, a second longitudinal side forming an illumination surface, and a first and second transverse side, an inlet well on an upper surface, an inlet channel, a sample processing channel in fluidic communication with the inlet channel and positioned substantially parallel to a longitudinal side, a sample splitter within the processing channel, a plurality of outlet channels in fluidic communication with the processing channel, and a plurality of collection wells in fluidic communication with each of the plurality of outlet channels wherein the substrate optionally comprises an optically transparent material and wherein the processing channel is offset within the plane of the of the substrate to be spatially biased to the imaging surface. The planar configuration allows for all required flowcell functions to be integrated into the cartridge and increases performance and reproducibility in a laboratory or clinical setting. In operation, it is critical for enhanced performance that the flow through the processing channel and into the outlet channel be as free of turbulence as possible. Effects of the differences in compressibility between air and liquid, channel configurations that may restrict flow, interact with sample solution meniscus or otherwise induce turbulent flow will reduce performance of particle separations. Minimization of flow conditions of a sample prior to entry into a processing channel can reduce sample loss and reduce opportunity for sample adherence and/or particle clumping within the channels of the flowcell as well as effects of sample handling on the viability of sample cells or organisms. The features of the present invention minimize these and other effects to improve performance and reproducibility. Where imaging within the flowcell is desired, the planar substrate comprises an optically transparent material. Glass, plastic, or polymer materials including cyclic olefin polymer (COP) or cyclic olefin copolymer (COC) are compatible for this application requirement. COP or COP can be utilized through precision injection molding. Other materials can be utilized that can form the cartridge through injection molding, etching, laser oblation, machining, or 3D printing. Typical dimensions of the planar substrate can be at least 50 mm in length, 20 mm in width, and at least 1.5 mm in thickness. Optional ranges are at least 100 mm in length, 35 mm in width, and about 2 to about 6 mm in thickness. The longitudinal sides of the cartridge act as waveguides for illumination and imaging. For that reason, the processing channel is offset in the plane of the substrate and is parallel and adjacent to the imaging longitudinal side of the substrate. Distances from the imaging side wall can be from about 0.5 mm to about 10 mm, preferably from about 0.5 mm to about 5 mm, optionally from about 1 mm to about 3.5 mm. In an embodiment the processing channel spacing from the imaging wall is about 2 mm. Channel dimensions for the processing channel can be any of the embodiments described previously herein. The volume of the processing channel can be configured from about 10 μL to about 800 uL, preferably from about 50 uL to about 600 μL, and optionally 100 uL to about 400 uL or about 150 uL to about 300 Ul. In some embodiments the volume is at least about 150 uL, at least about 200 uL, at least about 250 uL, or at least about 300 μL. The combined volume of the outlet channels must be greater that the volume of the processing channel. The flow volume split between two outlet channels can be an even split or can range from about 4:1 to about 1:4, about 3:1 to about 1:3, or about 2:1 to about 1:2 or can vary from 1:1 by about 50% or less, or about 40% or less, or about 30% or less, or about 15% or less when in operation in the system embodiments.

The flowcell of the present invention optionally includes collection wells on the planar substrate. The collection wells feature an inlet that is in fluidic communication with the outlet channel wherein the inlet is at a first well height and is configured with a step transitioning from the inlet port aperture to the floor of the well. The provides a transition surface for the flow of sample fraction into the well and can inhibit back siphoning of the sample fraction into the outlet channel and bubble formation within the collection well. An outlet channel within the collection well is provided with an opening that is at a height off the floor of the collection well that is higher than the opening of the inlet channel. The internal outlet can is placed in communication with a flow modulator, in some instances the flow modulator is an individual pump to provide flow through the flowcell. In operation, the collection well is sealed with a layer of material or film to provide an enclosed system to allow flow or pumping of sample and sample fractions through the flowcell. In assembling the flowcell layers and when an adhesive is used, it is important to provide a biocompatible adhesive. Correct adhesive selection is necessary to minimize or prevent leaching of adhesive components into the solution, adhering to cells or binding molecules from solution, being autofluorescent, having texture which increases the surface area and hence the impact on cells, and overly hydrophilic or hydrophobic. A preferred adhesive is a silicone or silicone-based adhesive.

### VII. Cell Separation System

A cell separation system of the present invention comprises a receiving block for retaining a flowcell cartridge, an optical system comprising an optical sensor, a lens, and an illumination source, and plurality of flow modulation components, wherein the receiving block removably places the flowcell cartridge in optical alignment with the optical system, removably engages a magnetic component adjacent to the processing channel of the flow cell, and removably places a plurality of outlet channels of the flowcell cartridge in fluidic communication with the plurality of flow modulation components. The optical is constructed to provide microscopic imaging of the processing channel of the above described flowcell cartridge. Optionally, the optical system is constructed and arranged to provide imaging for florescence emission with optional ultraviolet light exciter modules. The optical system may comprise a source of visible optical illumination constructed and arranged to provide light transmission through the processing channel within the planar substrate. The receiving block is constructed and arranged to hold the planar flowcell cartridge in an orientation to the optical system such that the imaging optics are aligned with the imaging side of the planar cartridge and the visible light emitter is in an orientation to illuminate the illumination side of the planar flowcell cartridge. Optionally, the optical system can further comprise one or more sources of ultraviolet illumination constructed and arranged to place the ultraviolet illumination, optionally at wavelengths of about 474 nm and/or 560 nm, in an angular orientation the imaging side of the planar cartridge to excite fluorophores within the processing channel for the cartridge.

For imaging of fluorescent entities internal to the processing channel optical system optionally comprises a dual bandpass filter preferably passing emitted radiation in bands centered at wavelengths at about 524 nm and 628 nm.

An optional feature of the receiving block is a series of flow modulator adapters that interface with outlets on the top or bottom of the flowcell cartridge. The adapters facilitate fluidic communication with flow modulators, such as a pump in the system, with outlet channels of the flow cells such as the collection well outlet channels. Once the flowcell cartridge is inserted into the receiving block, the receiving block is mechanically actuated to support the cartridge, aligning the illumination and imaging sides of the planar cartridge with the optical imaging system, align the magnetic components to the position them above and below the flowcell processing channel, and, where desired, place the flow modulator adapters in fluidic communication with corresponding outlet channels of the flowcell cartridge.

The flow modulators of the system provide flow to the sample and sample fractions within the flowcell cartridge. The flow rate provided by the flow modulators can range from as low as 1 uL per minute to as high as 1 mL per minute during separations. The flow rate can at or at least about 25 uL per minute, at or at least about 50 uL per minute, at or at least about 100 μL per minute, at or at least about 200 μL per minute, at or at least about 250 uL per minute, at or at least about 300 μL per minute, or from about 300 uL per minute to about 1 mL per minute. The total sample volume flowrate can be about 50 uL/min, about 75 uL/min, about 100 uL/min, about 150 uL/min, about 200 uL/min or about 300 ul/min. The flow volume split between two outlet channels can be an even split or can range from about 4:1 to about 1:4, about 3:1 to about 1:3, or about 2:1 to about 1:2 or can vary from 1:1 by about 50% or less, or about 40% or less, or about 30% or less, or about 15% or less when in operation in the system embodiments.

The magnetic components of the system may comprise materials, sizes, and strengths as described above and may be placed in configurations as described above and below.

### VIII. Cell and Nuclei Separation and Isolation

In a first aspect, the present disclosure provides a method (“Method 1”) for extracting cellular nuclei from intact cells comprising: providing intact cells; and lysing the cells in a nuclei isolating buffer; wherein the nuclei isolating buffer comprises wheat germ agglutinin (WGA). Wheat germ agglutinin (WGA) is a lectin that protects wheat from insects, yeast and bacteria. It is capable of blinding to glycosylated moieties such as N-acetyl-D-glucosamine and sialic acid, and is commonly used as in a fluorescently tagged state to label nuclei, and is routinely used for the staining of skeletal and cardiac sarcolemma to determine cross sectional area or myocyte density.

It has been discovered in accordance with the present disclosure that the inclusion of WGA in preparations for isolating nuclei from cells, and/or in preparations for isolating RNA from isolated nuclei, provides greater yields of RNA from the isolated nuclei.

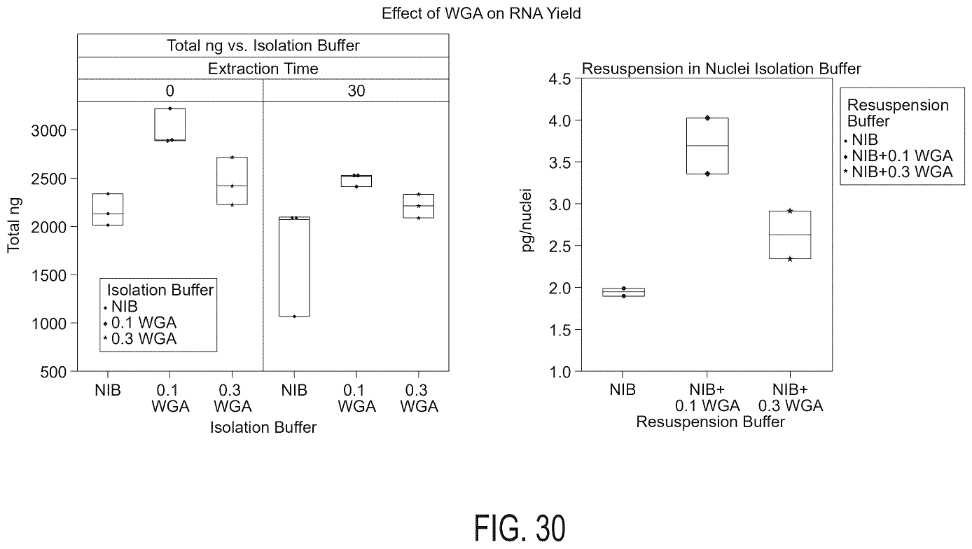

FIG. 30 shows results of performing bulk RNA extractions with and without WGA. Data are presented for total yield of RNA in nuclei isolation buffer, with and without WGA, and ng RNA/nuclei for the nuclei resuspended in isolation buffer, with and without WGA. It can be seen that the addition of WGA, particularly at 0.1 mg/mL, yields more RNA than the isolation buffer alone.

The nuclei isolating buffer can be any of the many known detergent containing buffer systems for isolating nuclei from cells. Typically, the WGA is present in an amount of from about 0.01 mg/mL to about 2 mg/mL; e.g., from about 0.01 mg/mL to about 1 mg/mL; e.g., from about 0.01 mg/mL to about 0.5 mg/mL; e.g., 0.05 mg/mL to about 0.15 mg/mL; e.g., about 0.1 mg/mL.

In a further embodiment, the method further comprises collecting the nuclei, e.g. by centrifugation. The nuclei can then be stored, for example by suspending the nuclei in a storage buffer; for example wherein the storage buffer comprises sucrose. In a further embodiment, the integrity of isolated cell nuclei is at least about 15% greater than the integrity of cell nuclei isolated by a similar method lacking WGA. In a further embodiment, the method further comprises suspending the collected nuclei in a levitation buffer containing a levitation agent. In a further embodiment, the levitation agent comprises 100 mM Gadolinium, and the levitation buffer comprises 1×PBS, 1% BSA, and a RNAse inhibitor, e.g. RNAseOUT™. In a further embodiment, the method further comprises performing any of the Methods 2 et seq. described herein utilizing the isolated nuclei.

In a second aspect, it has been discovered in accordance with the present disclosure that the presence of particles in the samples and/or flow stream of the apparatuses described herein can affect the movement of species in the sedimentation direction, in a size-dependent manner. The particles, referred to herein as “isolation particles” can be any of the bead type particles used for chromatographic separation, and can optionally be coated, for example with a protein. Examples of isolation particles include agarose beads, e.g. Sepharose™ beads, dextran beads e.g. Sephadex™, polyacrylamide beads, dextrose beads, polystyrene beads, beads made from polymeric resins. e.g. polyvinylethylcarbitol, polyvinylpyrrolidone, cellulose, silica-based materials, and/or from mixtures such as dextran-polyacrylamide, e.g. Sephacryl™ beads; each of which can optionally be coated, for example with a protein. In one particular embodiment, the isolation particles are polystyrene beads or particles; e.g. streptavidin coated polystyrene beads or particles, such as SPHERO™ Streptavidin Coated Particles sold by Spherotech, Inc., Lake Forest IL.

The present Methods 2 et seq. provide either positive or negative selection strategies for purification of cells and/or particles such as cell nuclei described herein. While not wishing to be bound by a particular theory, it is believed that the isolation particles may either a) form complexes or bind to non-nuclei components (i.e., contaminating species such as dead cells, cell fragments, aggregates of cell nuclei and/or other components, and the like) of the sample mixture; and/or b) form a matrix or barrier within the flow stream that traps or impedes free movement of contaminating species; either of which can affect the movement of species in the sedimentation direction, in a size-dependent manner. For example, it has been observed that the presence of 3 micron streptavidin coated polystyrene beads in the sample and/or flow stream, which levitate to the higher band positions in the flow stream, results in a negative-selection regime wherein non-nuclei components of the sample mixture are non-selectively retained among the streptavidin coated polystyrene beads in the higher bands of the flow stream, and the single nuclei filter through (i.e., sediment through) the streptavidin coated polystyrene beads and can be collected at a lower level in the stream. See Example 5, infra.

It will be appreciated that the present methods can also facilitate the isolation of a target subcellular component that is more buoyant (and/or more dense) than the isolation particles. For example, it is believed that selection of a less buoyant isolation particle would result in the isolation particles settling in a lower band of the flow channel, and target subcellular components that are more buoyant then the isolation particles residing in a higher band in the flow stream, which higher band could then be collected. Thus, the isolation of a desired target subcellular component could be facilitated by the selection of isolation particle of appropriate buoyancy and/or density in relation to the buoyancy and/or density of desired target subcellular component.

The method for separation of a mixture of live cells and dead cells comprises providing flowcell cartridge such as the flowcell cartridges of the embodiments above, comprising a processing channel, and a plurality of outlet channels wherein the combined volume of the outlet channels of the flowcell cartridge is a volume greater than the processing channel, flowing a sample solution comprising live cells and dead cells and a paramagnetic compound into the processing channel, placing the flowcell cartridge in a magnetic field substantially aligned parallel to the processing channel, maintaining the processing channel and the sample contained therein entirely within the magnetic field in a stopped flow condition for a period of time sufficient to separate live cells and dead cells by a vertical distance within the processing channel, simultaneously withdraw a sample fraction enriched with live cells and a sample fraction enriched with dead cells into the outlet channels. Optionally the method further comprises providing a flowcell cartridge that is substantially free of any liquid or paramagnetic compound prior to introduction of the sample solution.

The flowcell cartridge used in the methods of this invention may comprise outlet channels have a cross sectional area less than the cross sectional area of the processing channel and are arranged to follow compacted paths, one exemplary configuration being a serpentine channel. The magnetic field is placed in close proximity to the top vertical surface of the processing channel and in close proximity to the bottom vertical surface of the processing channel, each magnetic field have similar strength and surface field strength of between about 0.5 Tesla and about 2.0 Tesla and optionally between about 0.9 Tesla and about 1 Tesla. The surface field strength may be about 0.5 Tesla, about 0.6 Tesla, about 0.7 Tesla, about 0.8 Tesla, about 0.9 Tesla, or about 1.0 Tesla.

The method may further comprise providing a paramagnetic compound in the sample solution at a concentration of from about 50 mM to about 200 mM, optionally from about 65 mM to about 175 mM, and further optionally from about 70 mM to about 150 mM. The concentration may be about 70 mM, about 75 mM, about 80 mM, about, 90 mM, about 100 mM, about 110 mM, about 120 mM, about 130 mM about 140 mM, about 150 mM, about 160 mM, about 170 mM, about 180 mM, about 190 mM, or about 200 mM.

The method may further comprise the step of withdrawing the sample fractions into the outlet channels at a flow rate of from about 75 uL per minute to about 150 uL per minute, and optionally at about 75 uL per minute, about 90 uL per minute, about 100 μL per minute, about 110 uL per minute, about 120 uL per minute, or about 150 uL per minute.

The method produces exceptional recovery and purity of live cell fractions. The enriched recovered sample fraction comprises at least about 60%, at least about 70%, at least about 80% or at least about 90% live cells and the yield of live cells in the enriched recovered sample fraction is at least about 50%, at least about 60%, at least about 70%, or at least about 75% of the total live cell composition of the sample.

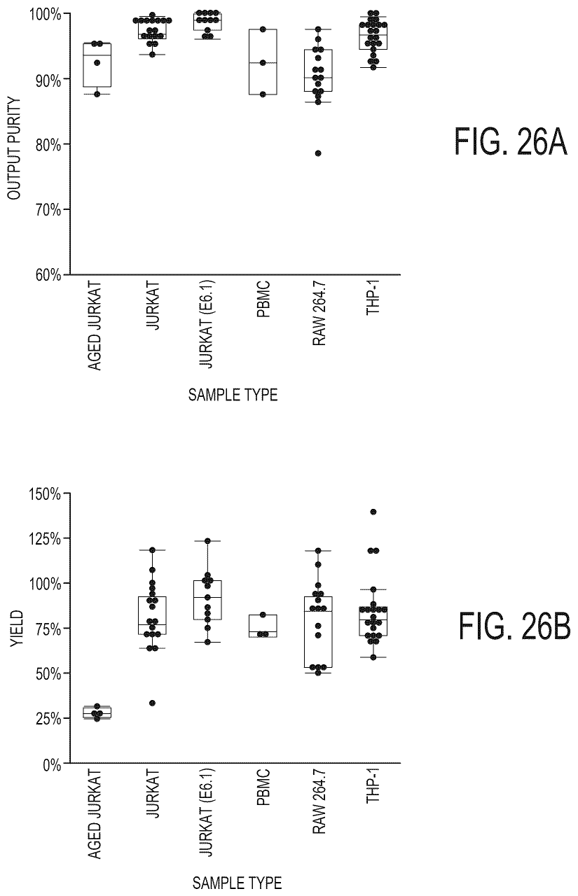

Experiments were performed to separate live and dead Jurkat cells on different instrument with different flowcell configurations. A population of dead Jurkat cells was created by treating fresh Jurkat cells with 70% ethanol. After removing the ethanol and washing the dead cells in media, a mixed population was created by mixing the dead cells back into the original live cell population. The concentration of dead cells was approximately 20% in this final mixture. Aliquots of the cell mixture were separated using the flowcells and systems as described herein using conditions optimized for each instrument configuration. FIGS. 25A and 25 B show live cell fraction viability and live cell yield obtained by the method described herein. The ability to provide efficient live cell separations on a variety of cell types by the methods described herein is demonstrated in FIGS. 26A and 26B. Live cell separation and enrichment has been performed on primary cell, primary cells isolated from human and animal tissue, dissociated cells from tumors, cultured cells, and other cells as described herein.

Isolation of nuclei or particles is rapidly accomplished without subjecting the nuclei to the stresses associated with other washing and/or separation techniques such as FACS or wash/centrifugation. Rapid isolation of nuclei is performed by a method comprising; loading a sample comprising cell nuclei and a sample medium comprising a paramagnetic compound or ferrofluid into a separation channel, subjecting the sample to a magnetic force with at least one magnet to affect a separation, collecting at least one fraction of the separated sample comprising cell nuclei without further centrifugation, and optionally imaging the particles in the sample prior to, during, and/or after the separation. In this method, the sample can comprise from about 50 to about 10,000,000 nuclei. The total time for separation can range from about 1 minute to about 20 minutes. In the rapid separation method, the concentration of cell nuclei of interest is increased in the solution or within a portion of the solution by a ratio of at least about 100:1, 90:1, 80:1, 70:1, 60:1, 50:1, 40:1, 30:1, 20:1, 10:1, 5:1, 3:1, 2:1, 1.5:1, or 1.1:1. Cell nuclei within a solution containing multiple types of particles may be deemed “separated” if, after processing the solution, the ratio of the cell nuclei of interest to the concentration of other types of particles is increased, or if the ratio of the concentration of the cell nuclei to the concentration of other types particles is increased by at least about 10%, 50%, 100%, 200%, 300%, 400%, 500%, 600%, 700%, 800%, 900%, or 1000%, or if the concentration of non-nuclei particles in the solution is decreased by at least about 20%, 15%, 10%, 8%, 6%, 5%, 4%, 3%, 2%, 1%, or 0.5%. In a preferred embodiment of the cell nuclei enrichment method, the integrity of isolated cell nuclei in the enriched collected portion of the sample is greater than 30% of the integrity of similar cell nuclei isolated by a method comprising centrifugation. Preferably the integrity of isolated cell nuclei in the enriched collected portion of the sample is at least 35%, 40%, 45%, 50%, 55%, 60%, 65%, 70%, 75%, 80%, 85%, 90%, 95%, 97%, or 99% of the integrity of similar cell nuclei isolated by a method comprising centrifugation. Integrity is defined integrity of the nuclear membrane resulting in containment of a substantial proportion of DNA and RNA within the isolated nuclei. The starting sample may comprise from about 50 to about 10,000,000 cell nuclei, from about 500 to about 10,000,000, from about 2,000, to 10,000,000, from about 10,000 to about 10,000,000, from about 25,000 to about 10,000,000, from about 50,000 to about 10,000,000, from about 75,000 to about 10,000,000, from about 100,000 to about 10,000,000, from about 150,000 to about 10,000,000, from about 200,000 to about 100,000,000 or from about 500,000 to about 10,000,000 particles. The time to separate and collect fractions of the separated particles can be 20 minutes or less, 18 minutes or less, 15 minutes or less, 12 minutes or less, 10 minutes or less, 8 minutes or less, 5 minutes or less, 3 minutes or less, or less than 1 minute. The cell nuclei types separated by the above method can include human cells, non-human animal cells, plant cells, eukaryotic cells (for example, but not limited to, immune cells, endothelial cells, yeasts and T-cells). Isolated nuclei can be from uninucleate, bi-nucleate, multinucleate of enucleate cells. Multiple cell types may include dead cells, alive cells, healthy cells, pathological cells, infected cells, transfected cells, or genetically modified cells. Cell nuclei isolated according to the methods of this disclosure can be directly obtained from an organism, or from propagated or cultured cells. In a particular embodiment of the rapid, high capacity separation method, live cells are separated from dead cells. In another embodiment of the isolation method, nuclei are isolated from viable transfected cells and/or crispr modified or otherwise gene-edited cells. In another embodiment of the method, cell nuclei are separated from cells, cell fragments, and/or sample debris. In various embodiments, these methods are performed utilizing the cartridges and systems described herein.

### Well-Based Embodiments

In one embodiment, the System described herein includes a levitation sample fixture that includes a) a removable multi-well plate comprising a top surface and a plurality of optically transparent wells; b) a magnet array comprising a plurality of magnets disposed in between and below the wells, configured to provide a magnetic field in each of the wells; and c) a magnet holder configured to receive and hold the magnets of the magnet array. In further embodiments, the levitation sample fixture can include a mirror assembly for projecting cross-section images of the wells vertically downwards to an image-capturing device. In further embodiments, the levitation sample fixture can include a plurality of metal pins configured to attenuate and/or adjust the magnetic fields in the wells. In further embodiments, the System can include an imaging array for capturing images from the mirrors. In further embodiments, the System can include a fluid transfer system, i.e., means to dispense or remove all or part of a sample or other fluid into or out of the wells; e.g., a pipetting robot.

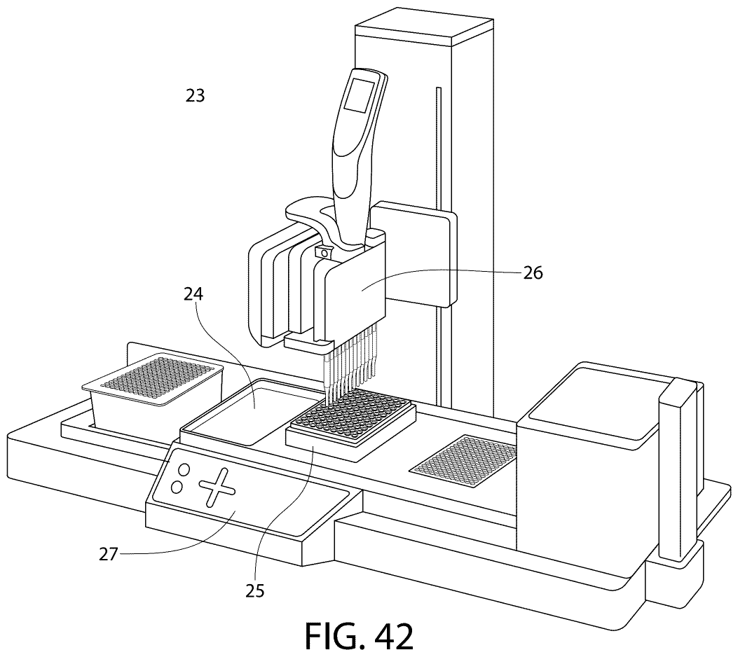

In further embodiments, the System can further include a deck or stage holding the levitation sample fixture, the imaging array and the fluid transfer system. FIG. 42 depicts one exemplary System (23) comprising a deck or stage (24) adapted to engage and hold levitation sample fixture (25). The System further includes fluid transport system (26) and control panel (27). In further embodiments, the System can include means to move the multi-well plate in a vertical direction relative to the fluid transfer system; and/or means to move the levitation sample fixture and/or the multi-well plate in a vertical direction relative to the magnet array. In some embodiments, the movement of the levitation sample fixture and/or multi-well plate in a vertical direction is accomplished by mounting the levitation plate (levitation sample fixture and/or the multi-well plate) on a single- or multi-axis mechanism, e.g. a motion stage. The mechanism can be driven, for example, mechanically, e.g. via a motor and lead screw, or a belt drive, or a via way to store energy such as a loaded spring: manually; by piezoelectric drive by direct or indirect means; pneumatically; hydraulically; or using magnetic forces.

### A. Multi-Well Plate

The multi-well plate is a removable plate having a top surface and is configured to contain a plurality of optically transparent wells. Such plates are well known and are used in numerous applications such as PCR. In some embodiments, the dimensions and well configuration of the multi-well plate are in accordance with ANSI SLAS microplate standards; e.g., with a footprint of 127.76 mm×85.48 mm+0.5 mm. The multi-well plate can have any standard number of sample wells—e.g., 6, 12, 24, 48, 96, 384 or 1536 sample wells, preferably arranged in a 2:3 rectangular matrix. In one embodiment, the multi-well plate has 96 sample wells arranged in a 2:3 rectangular matrix.

In some embodiments, the well positions of the multi-well plate is in accordance with ANSI SLAS microplate standards; e.g., wherein 96-well plates have a 9 mm well-to-well spacing, 384-wells a 4.5 mm spacing, and 1536-wells a 2.25 mm spacing. In some embodiments, the multi-well plate has the footprint and/or wall thickness of a standard 96-well PCR plate. The wells of the multi-well plate can be round or square, and have various standard geometries at the bottom of the well, including F-Bottom, V-Bottom, U-Bottom and C-Bottom geometries.

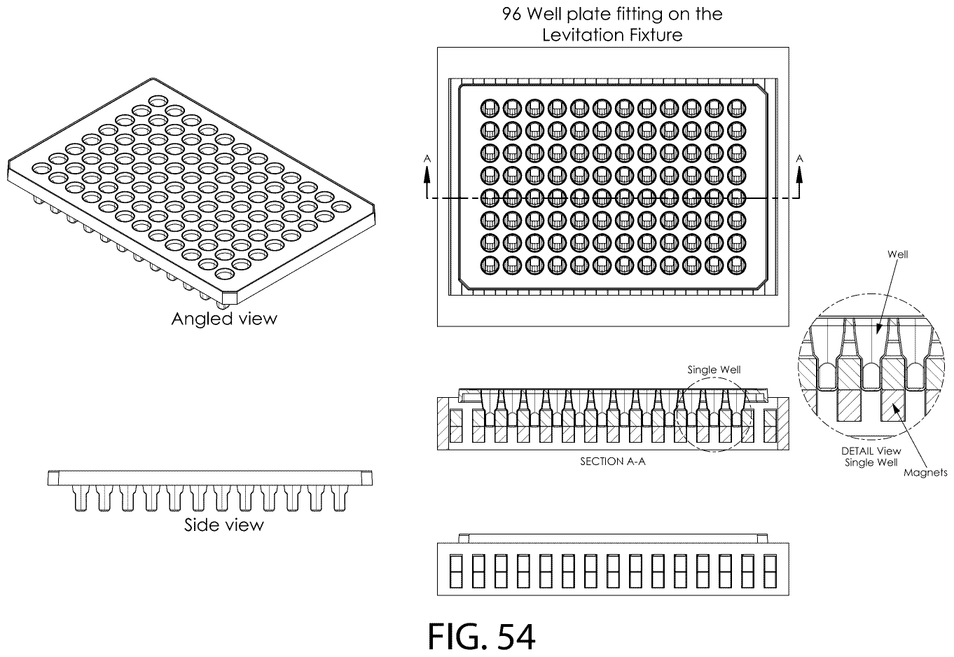

FIG. 54 shows an example of a multi-well plate having wells with increased volumes. In this example, the plate would comprise 96 wells, with volume between 200 and 10,000 uL, or more preferably between 500 and 2,000 uL

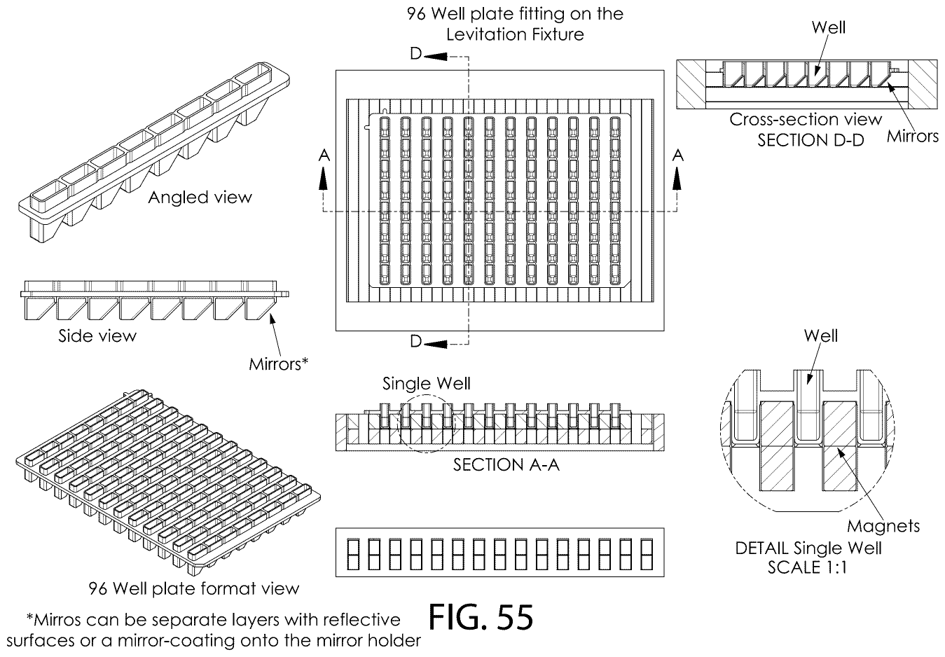

In some embodiments, the mirrors can be incorporated into the multi-well plate, obviating the need for inclusion of a mirror holder. FIG. 55 shows an example of such a design.

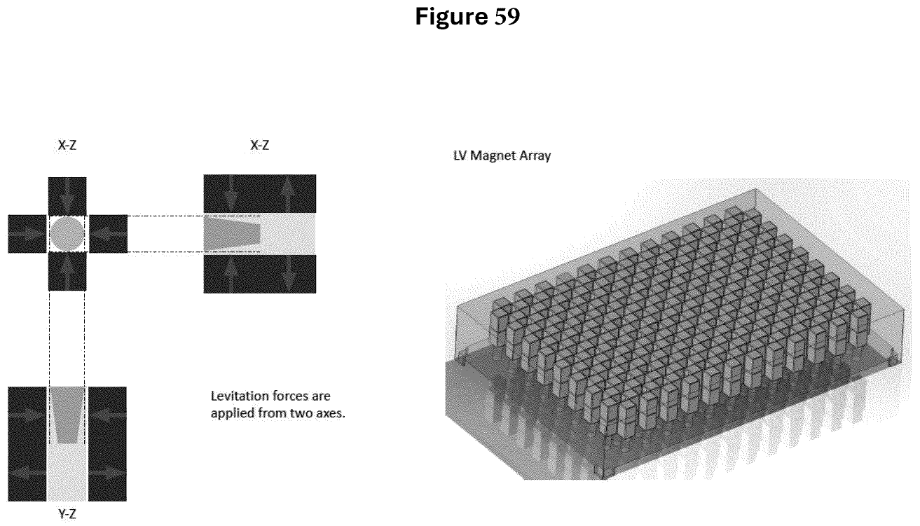

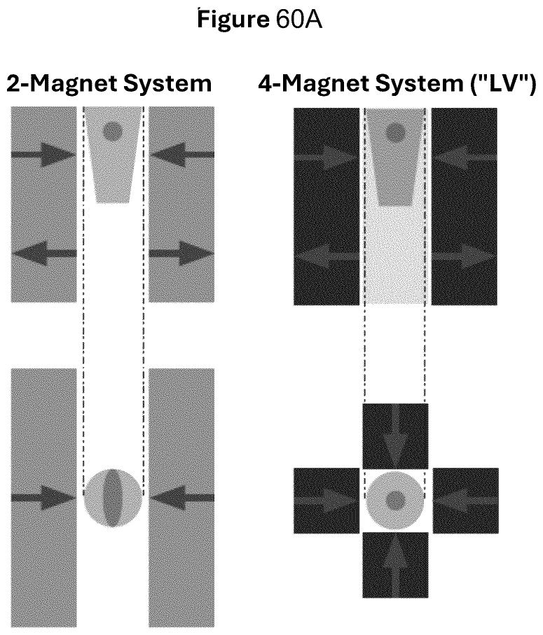

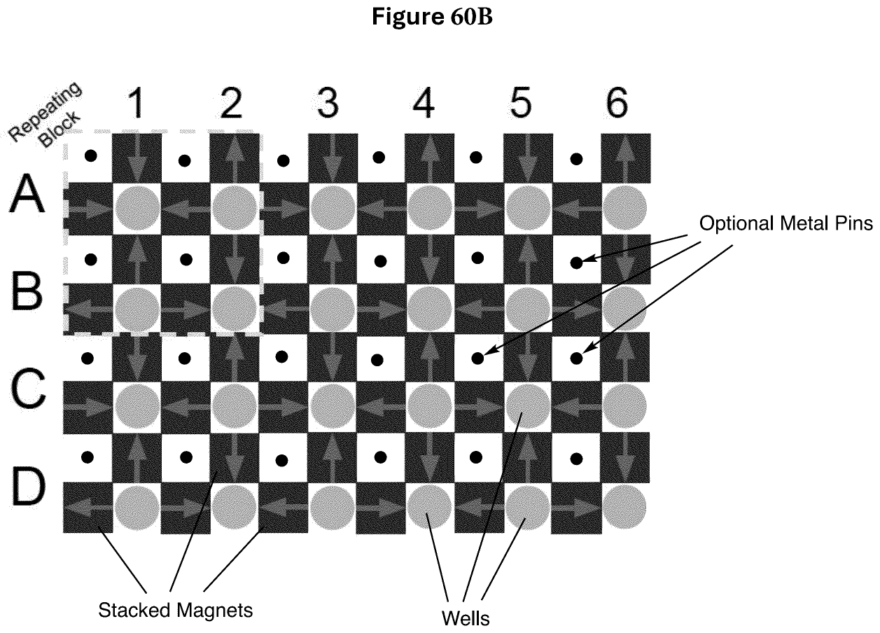

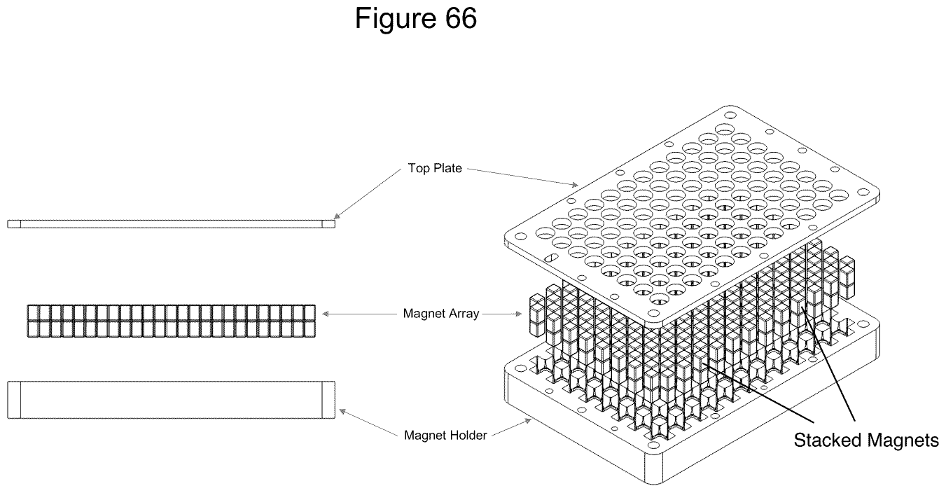

FIG. 66 shows an exploded view of one embodiment of a LV Array according to the present disclosure. The stacked Magnet Array, e.g. as shown in FIGS. 59 and 60B, fits into mating spaces in the Magnet Holder. The Magnet Array is covered by a Top Plate, which contains holes for accommodating the wells, for example the wells of a 96-well plate as described above.

### B. Magnet Array/Magnetic Field

The present disclosure provides for methods and devices for concentrating using a magnetic field within a well of a multi-well plate. The interaction of the magnetic field with the paramagnetic medium provides separation of components in the sample according to density of the species.

In accordance with one preferred embodiment, the magnets are permanent magnets. In accordance with one embodiment, the maximum energy product of magnets range from about 1 Mega-Gauss Oersted to about 1000 Mega-Gauss Oersted, and more preferably ranges from about 10 Mega-Gauss Oersted to about 100 Mega-Gauss Oersted. In accordance with an embodiment, the surface field strength of magnets range from about 0.1 Tesla to about 100 Tesla, and more preferably ranges from about 1 Tesla to about 10 Tesla. In accordance with an embodiment, the remanence of magnets range from about 0.5 Tesla to about 5 Tesla, and more preferably ranges from about 1 Tesla to about 3 Tesla.

In accordance with a preferred embodiment, magnets are made from a material comprising neodymium alloys with iron and boron, neodymium, alloys of aluminum with nickel, neodymium alloys with iron, aluminum and cobalt alloyed with iron, samarium-cobalt, other alloys of rare earth elements with iron, alloys of rare earth alloys with nickel, ferrite, or combinations thereof. In accordance with an embodiment comprising a plurality of magnets, magnets are made from the same material or are made from different materials. In some embodiments, suitable magnets include N50-N53 magnets, for example N52 magnets. In some embodiments, the magnet fixtures are 83 mm×6.4 mm×4.6 mm.

### C. Exemplary Magnet Configurations

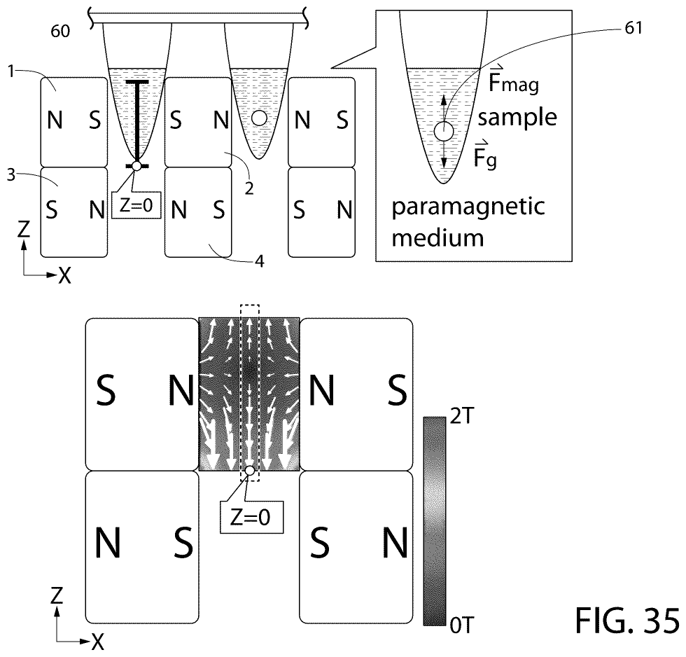

Stacked linear array: In accordance with a preferred embodiment, for each well, a first magnet and a second magnet are disposed adjacent to the well to impart a magnetic field that is inside the well; wherein each magnet comprises a first pole and a second pole, and the first pole of the first magnet faces the first pole of the second magnet; and a third magnet is stacked under the first magnet, and a fourth magnet is stacked under the second magnet, wherein the third and fourth magnets each comprise a first pole and a second pole, and wherein the second pole of the third magnet faces the second pole of the fourth magnet. In some such embodiments, the magnets are rectangular. In one exemplary embodiment, the dimensions of the magnets are 83 mm×6.4 mm×4.6 mm. An exemplary depiction of this configuration is shown in FIG. 35, where magnets 1, 2, 3 and 4 correspond respectively to the first, second, third and fourth magnets described above. As shown in the upper panel, well (60) is positioned such that the bottom of well (60) is at the interface of upper magnets (1)/(2) and lower magnets (3)/(4), with the equilibrium levitation position of the levitated sample (61) occurring at a positive distance z above the bottom of the well. The lower panel shows the magnetic field strength relative to the magnets.

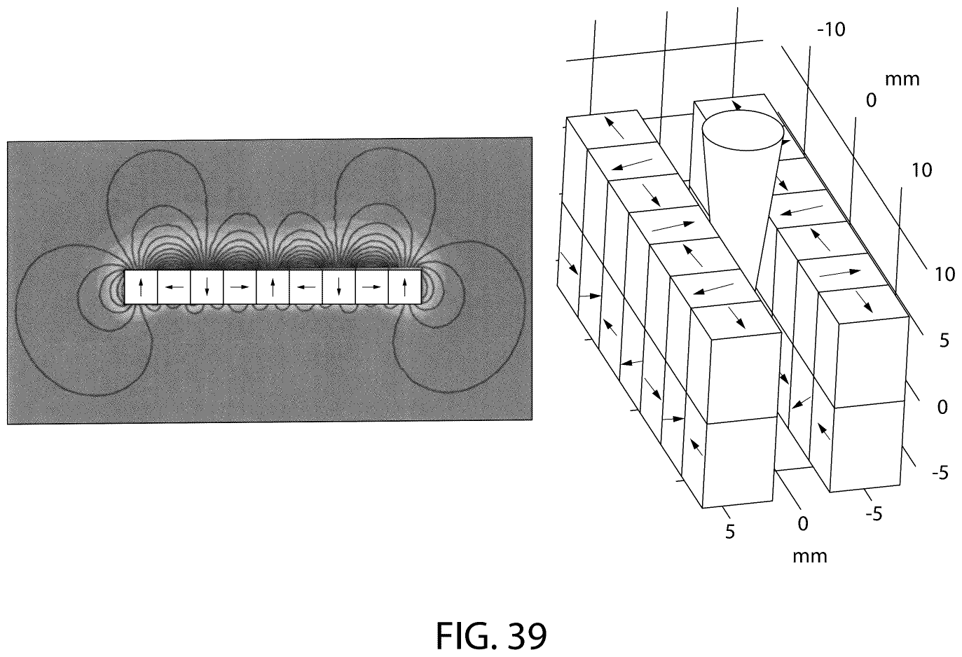

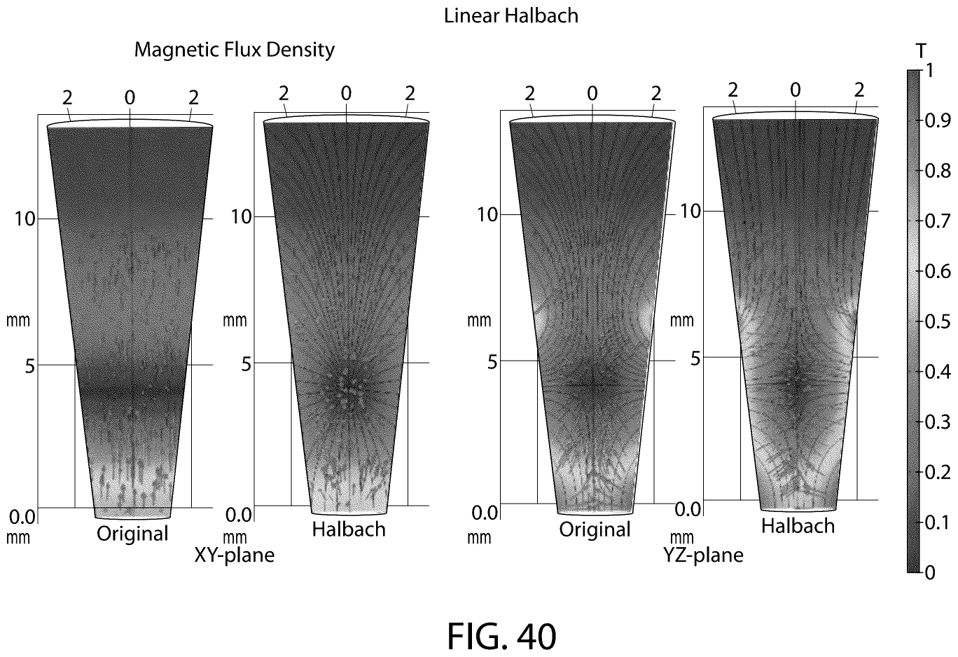

Linear Halbach array: In accordance with a further embodiment, the magnets can be arranged in a linear Halbach array, which has a spatially rotating pattern of magnetization and results in augmentation the magnetic field on one side of the array while cancelling the field to near zero on the other side. The Halbach configuration adds a magnetic gradient component in the XY plane (parallel to the bar magnets) and the YZ plane, focusing the sample in two planes. An exemplary depiction of this configuration is shown in FIG. 39, and a simulation comparison of the magnetic flux density in the stacked linear array and the Halbach array is shown in FIG. 6. As can be seen in FIG. 40, the Halbach array provides a stronger magnetic field near the bottom and the walls of the well.

Four-Stacked Magnet (“LV”) Array: In one preferred embodiment, the stacked linear array contains four sets of stacked magnets surrounding each well. In one such embodiment, a first magnet and a second magnet are disposed adjacent to, and on opposite sides of, the well to impart a magnetic field that is inside the well; wherein each magnet comprises a first pole and a second pole, and the first pole of the first magnet faces the first pole of the second magnet; a third magnet is stacked under the first magnet, and a fourth magnet is stacked under the second magnet, wherein the third and fourth magnets each comprise a first pole and a second pole, and wherein the second pole of the third magnet faces the second pole of the fourth magnet; a fifth magnet and a sixth magnet are disposed adjacent to, and on opposite sides of, the well, to impart a magnetic field that is inside the well; wherein each magnet comprises a first pole and a second pole, and the first pole of the fifth magnet faces the first pole of the sixth magnet, and wherein the fifth and sixth magnets are oriented at approximately 90 degrees in the horizontal plane relative to the first and second magnets; and a seventh magnet is stacked under the fifth magnet, and an eighth magnet is stacked under the sixth magnet, wherein the seventh and eighth magnets each comprise a first pole and a second pole, and wherein the second pole of the seventh magnet faces the second pole of the eighth magnet. FIG. 59 in the right-hand panel shows an exemplary configuration of the magnet array. FIG. 60B shows an overhead view of the array, and a depiction of the magnetic forces generated around the wells.

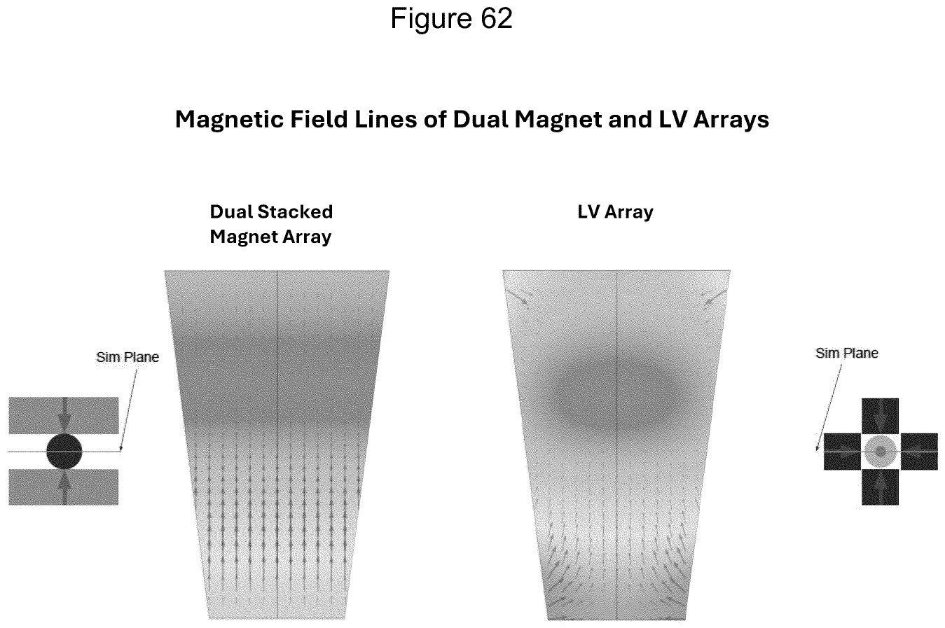

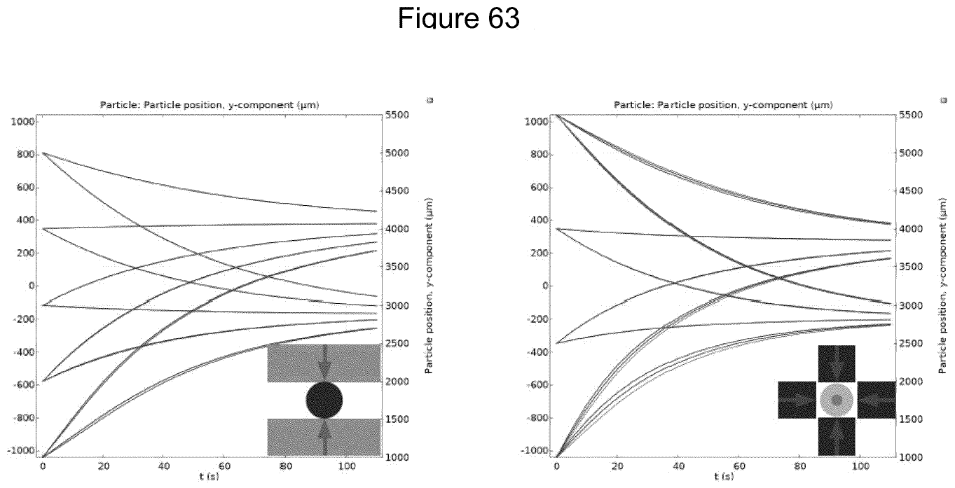

FIGS. 59 and 60A shows the effect of magnetic forces from the system containing two stacked magnets surrounding each well, and the system containing four stacked magnets surrounding each well (the latter being designated the “LV” system). As can be seen, the presence of the additional two stacked magnets results in force being applied from four direction, with the result that the sample is levitated into a shape that is more spherical and less ellipsoid relative to the 2-stacked magnet configuration. This can also be seen in FIG. 62, which shows the magnetic field lines of dual stacked magnet and LV arrays. It can be seen that the LV configuration results in greater focusing into the central portion of the well, including at the corners, where the field of the 2-stacked magnet configuration appears to be less strong. FIG. 63 shows a comparison of the particle height and response time for an exemplary dual stacked magnetic array of the present disclosure, and a LV Array of the present disclosure. It can be seen that the LV array results in a comparable focused particle height in the wells, and also has a similar response time.

In some embodiments, the LV array contains a plurality of blocks that each contain four wells, and their associated stacked magnets. Such a repeating block is shown in FIG. 60B.

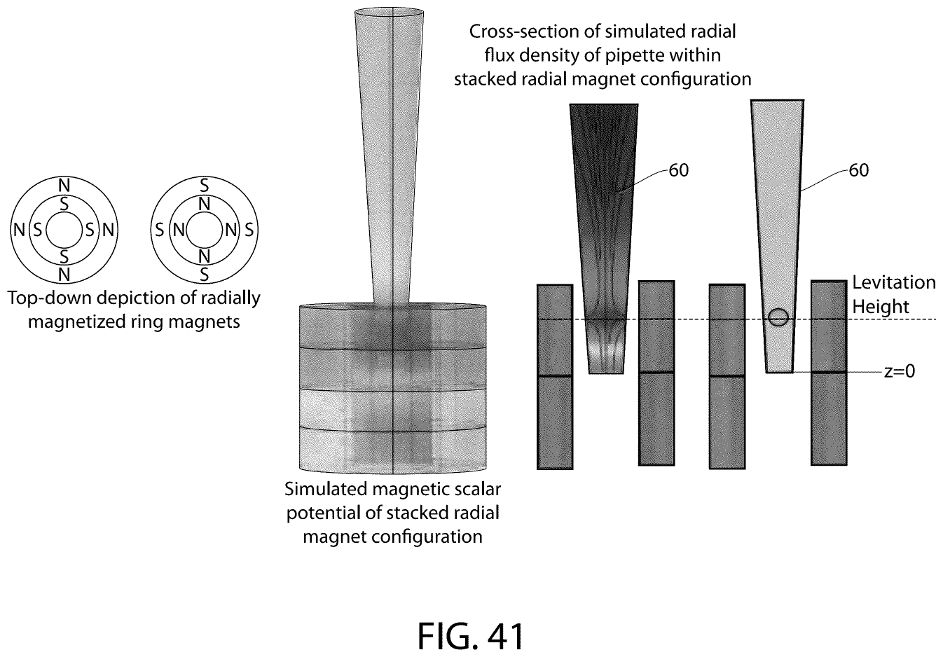

D. Ring magnet array: In accordance with a further embodiment, the magnets of the array can be ring magnets that surround the wells. The ring magnets can be axially magnetized or radially magnetized. In one embodiment, two ring magnets are stacked one upon the other and are disposed around one or more wells, for example all the wells, of the multi-well plate. Preferably, the poles of the stacked magnets are radially opposed. As shown in FIG. 41, the radially opposed magnets are configured to create a strong gradient at the interface with a low field zone in the height of the upper magnet, which creates a trap that confines particles within the lowest magnetic field.

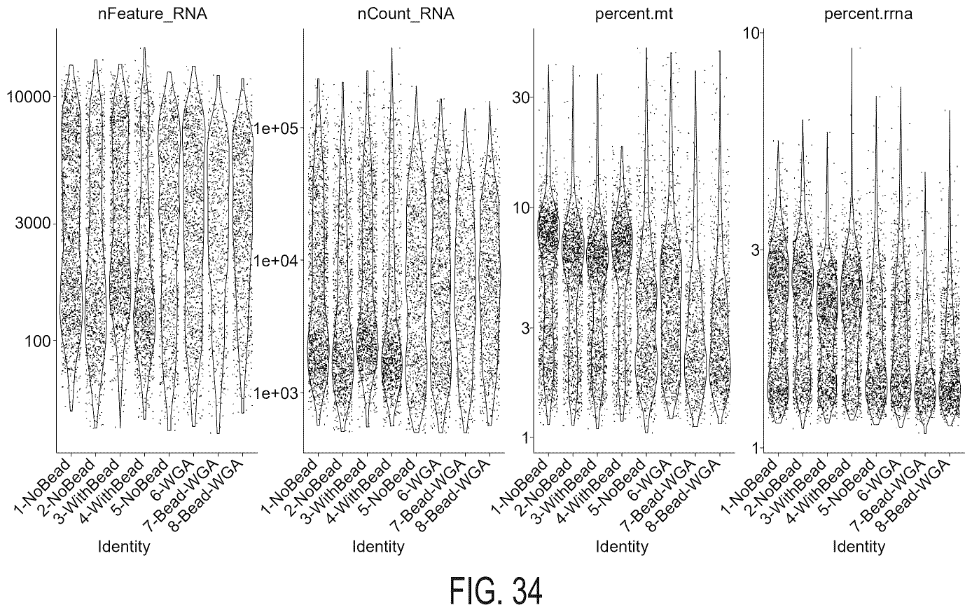

In some embodiments, the magnets of the array that are adjacent to the wells (i.e., in a linear array), or are surrounding the wells (i.e., in a ring magnet array), comprise two stacked magnets. As shown in FIGS. 34 and 41, in some embodiments, the bottom of the wells (60) (z=0) is positioned at the interface of the stacked magnets for the levitation procedure, and the levitated species, e.g., live cells, levitate to a point at a positive distance z above the bottom of the well, where the magnetic field is lowest.

In accordance with an embodiment, an asymmetric magnetic field is achieved by using a stronger magnetic material on one side of a multi-well plate well and a weaker magnetic material on the opposite side of the multi-well plate well. In accordance with a preferred embodiment, an asymmetric magnetic field is achieved by using a magnetic material on one side of a multi-well plate well and a substantially similar magnetic material on the opposite side of the multi-well plate well. In accordance with such embodiment, the upper magnet and lower magnets may be substantially the same size. In accordance with such embodiment, upper magnet may comprise neodymium, lower magnet may comprise samarium-cobalt, and wherein both magnets are substantially the same size. Alternatively, upper magnet may comprise samarium-cobalt, lower magnet may comprise neodymium, and wherein both magnets are substantially the same size.

The magnet holder forms the base of the levitation sample fixture and is configured to receive and hold the magnets of the magnet array, for example rectangular magnets and ring magnets, and provides the support for the other components of the levitation sample fixture. In some embodiments, the magnet holder is made of non-ferrous metals such as aluminum or titanium, structural plastics such as polyoxymethylene or

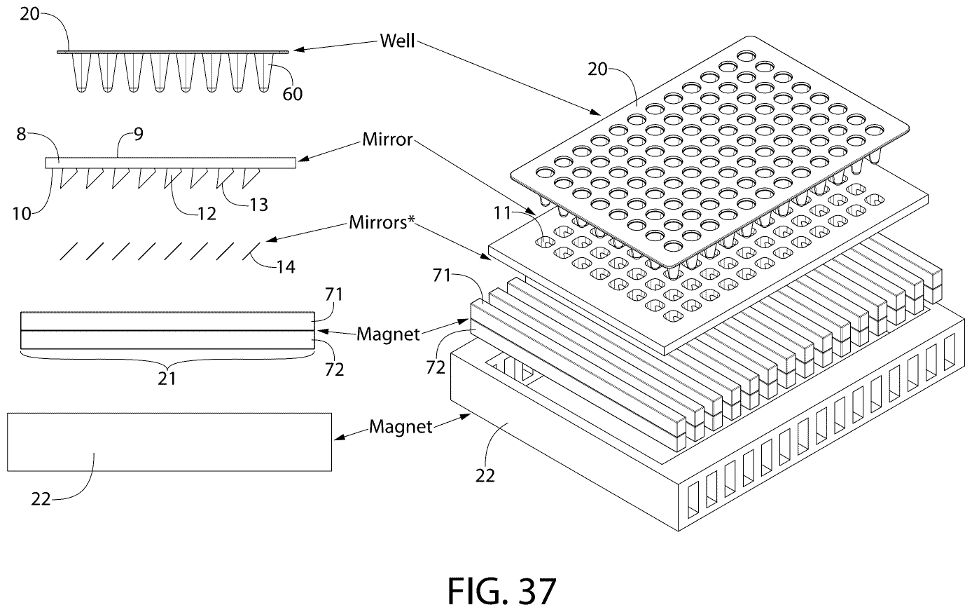

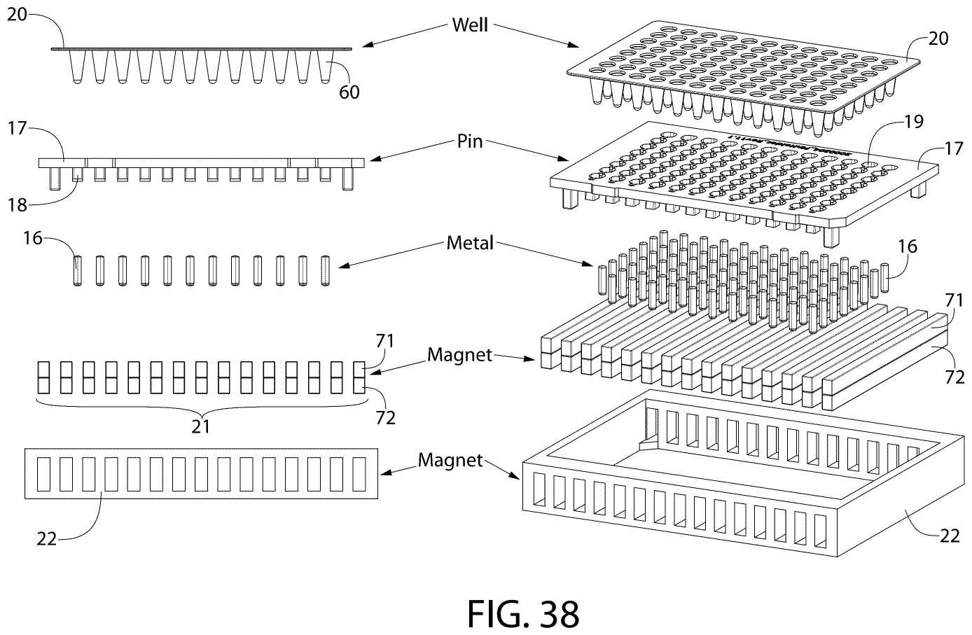

E. Magnet Holder PEEK, and hardwoods. In some embodiments, the magnet holder has same footprint dimensions as a standard multi-well plate, i.e., of 127.76 mm×85.48 mm; +0.5 mm. Exemplary depictions of the magnet holder (22) are shown in FIGS. 37 and 38.

### F. Mirror Assembly/Mirrors

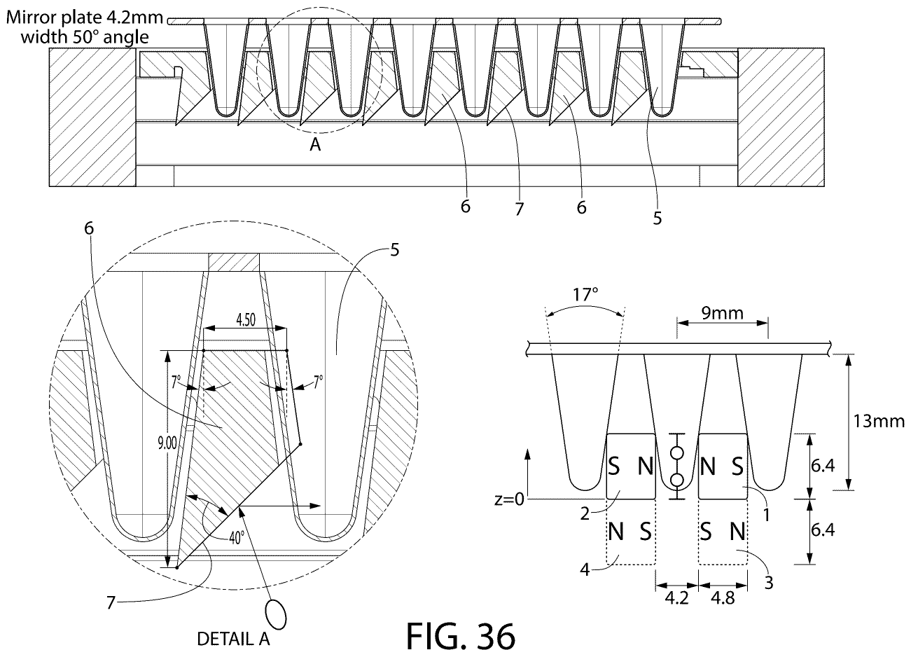

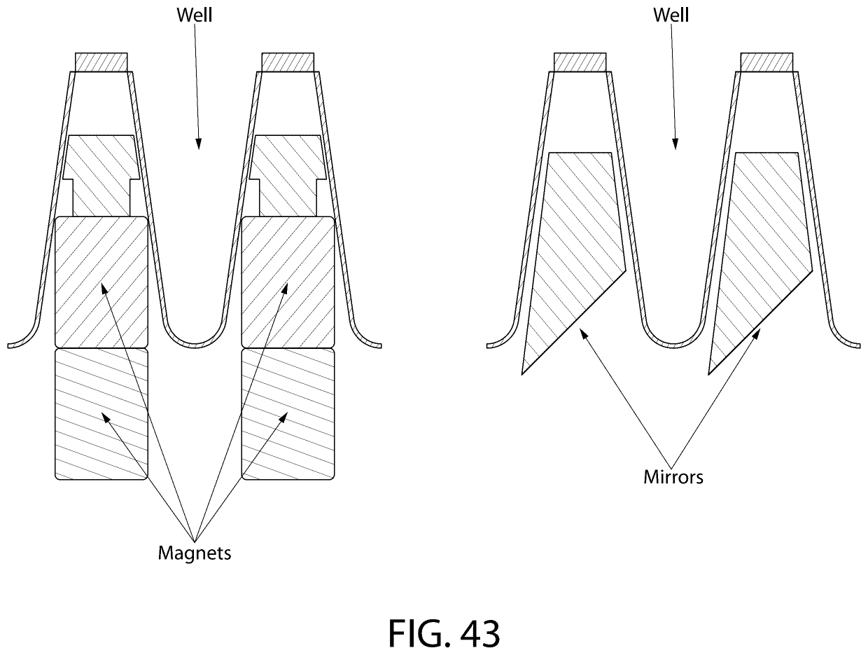

In some embodiments, the levitation sample fixture further includes a mirror assembly containing mirrors that are positioned to project, substantially parallel to the top surface of the plate, cross-sectional images of the wells along their vertical axes; and optionally, a mirror holder. In some preferred embodiments, the mirrors are positioned at about 40° to about 50° with respect to the vertical axis substantially perpendicular to the main surface of the multi-well plate. Exemplary exploded views of such embodiments are shown in FIGS. 2 and 3, and cross-sectionals view of wells showing exemplary placement of the mirrors and magnets is shown in FIGS. 36 and 43.

FIG. 37 depicts an exemplary levitation sample fixture (24) that comprises a mirror array. In this embodiment, shown in FIG. 37, the mirror holder is a single plate (8) having a top surface (9) and a bottom surface (10), with holes (11) configured to accommodate the wells (60) of the multi-well plate. The mirror holder (8) has protrusions (12) that extend down from the bottom surface (10) of the mirror holder that include a planar surface (13) upon which a mirror (14) or a reflective coating is disposed. The protrusions are configured to provide placement of mirrors adjacent to the wells (60), and to provide that the angle of the mirror or reflective coating with respect to the vertical axis substantially perpendicular to the main surface of the multi-well plate is about 40° to about 50°. The protrusions (12) and mirrors (14) of the mirror holder (8) extend downward into the magnet array (21). The magnets (71) and (72) of the magnet array (21) are held by magnet holder 22. In one embodiment, the mirrors are configured substantially in accordance with FIG. 36, which shows the placement of mirror holder (6) and mirrors (7) relative to wells (5) and magnets (1)-(4), as well as exemplary dimensions for one embodiment of the wells and magnets. In further embodiments, mirrors or reflective coatings are present only at selected wells, individually, or in rows and/or columns. In some such embodiments, mirrors or reflective coatings are present in one row of wells and/or one column of wells, or more than one row and/or column of wells, up to all of the rows and/or columns of wells.

In one embodiment, the mirrors are contained in a separate layer having a reflective coating. For example, the separate layer provides a support surface for reflective material, e.g. reflective adhesive-backed foil, to be added. The support surface is configured such that the desired view of the wells is achieved, by adding foil as individual pieces or long strips, providing for advantages during manufacturing.

### G. Metal Pin Containing Embodiments

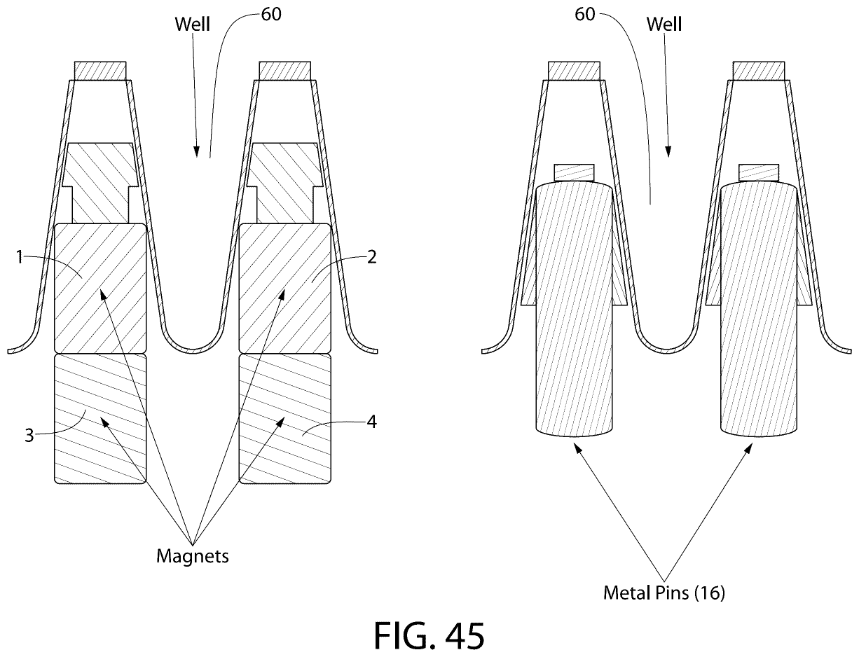

In some embodiments, the levitation sample fixture further includes a plurality of metal pins configured to attenuate and/or adjust the magnetic fields in the wells. Preferably, the pins comprise or are made of a material with high magnetic susceptibility such as steel, iron, cobalt, nickel, and/or alloys such as magnesium alloy, nickel alloy, and chromium alloy. In one embodiment, the pins are steel rods. In some embodiments, the pins are cylindrical. In further embodiments, the pins are in the shape of posts with square cross-section. In a preferred embodiment, the pins are disposed in between the wells and are configured to increase the magnetic field gradients within the wells. FIG. 38 is an exploded view of a levitation sample fixture (15) according to the present disclosure comprising metal pins. In this embodiment, the metal pins (16) are held by a pin holder (17) that has posts (18) to accept pins (16), and holes (19) configured to accept the wells (60) of well plate (20). The metal pins are disposed in between the magnets (71) and (72) of magnet array (21), which in turn are held by magnet holder (22). A cross-sectional view of a well, showing the placement of magnetic pins (16) relative to the well (60) and the magnets (1)-(4) is shown in FIG. 45.

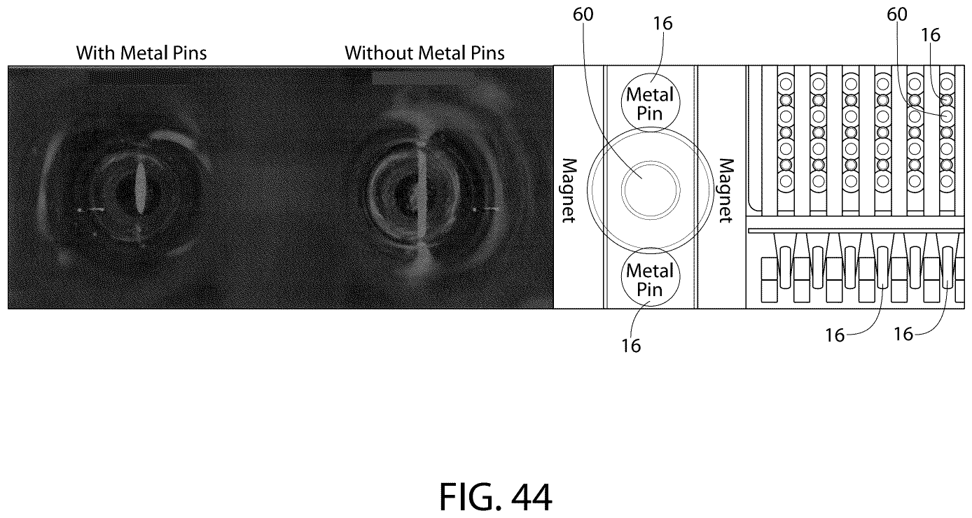

FIG. 44 shows one exemplary placement of the magnetic pins in between the sample wells (60). As a result of the perturbation of the magnetic field by the pins on the equilibrium position of levitated species, the embodiment with metal pins provides levitation of cells and/or other particles that is more centered in the well, and does not extend to the walls of the cells. In the embodiment without pins, lacking the magnetic field perturbation, the equilibrium position of the isolated species is less centered in the well, and extends from the walls of the wells, which makes collection more difficult.

FIG. 60B shows an exemplary placement of the pins in a LV array, with the pins placed diagonally in between the sample wells. While not wishing to be bound by a particular theory, it is believed that the presence of the pins results in increased field focusing in the corners, resulting in more rapid and/or stronger focusing.

In one embodiment, two of the stacked magnets surrounding each well of the LV array can be replaced by metal pins as described above. While not wishing to be bound by a particular theory, it is believed that in some embodiments, the pins will strengthen the magnetic fields in the wells in a similar fashion to the stacked magnets. For example, referring to FIG. 60B, the stacked magnets in Rows A, B, C and D could be replaced with pins, with or without the presence of the optional metal pins diagonal to the wells; or the stacked magnets in Rows 1, 2, 3, 4, 5 and 6 could be replaced with pins, with or without the presence of the optional metal pins diagonal to the wells.

### H. Imaging Array

In some embodiments, the System further includes a visualization component or imaging array. The visualization component may comprise any device which enables or enhances the ability to view in real time and/or to record the levitation of the desired species in the wells as the levitation proceeds, thereby enabling observation and/or measurement of the isolation of the desired species, including the extent of isolation and/or the rate of isolation. Visualization may also include analysis of the size, shape, or other characteristics of the particles and/or other components of the sample. The visualization system may employ optics to allow bright-field illumination, dark-field illumination, and/or fluorescent detection of sample components.

In one embodiment, the visualization component is an imaging array disposed beneath the multi-well plate and magnet array. In one embodiment, the visualization component comprises a microscope, e.g., a USB microscope, and means for moving the microscope underneath the multi-well plate from well to well, for example a motorized stage, manual placement, or pneumatic positioners. In a further embodiment, the imaging array comprises a camera array comprising a plurality of cameras, and means for moving the cameras underneath the multi-well plate from well to well, for example a motorized stage. In one embodiment, the camera array is a motorized camera array. In some such embodiments, the plurality of cameras comprises one or more of built-in lenses, motorized focus, and zoom-in capability. In one embodiment, each camera is configured to capture images from a plurality of the wells. In a preferred embodiment, each camera is configured to capture images from one to twenty-four of the wells; for example from four wells or from six wells.

In one embodiment, the visualization component further comprises a graphics processing unit (GPU) comprising graphics software that can integrate two or more of the images from the cameras. In some embodiments, the graphics software can integrate up to all of the images from the cameras, providing an array of up to all the images from up to all of the wells, in a single graphic. Integrating multiple images can also consist of one or more of: merging images into a single larger image, combining images from multiple times in a single data structure to allow for analysis of differences over time, or collating a subset of images for the purposes of a report.

### I. Fluid Transfer System

In some embodiments, the System further includes a fluid transfer system for transferring fluids into and out of the wells. In one embodiment, the fluid transfer system includes means to dispense or remove all or part of a sample or other fluid into or out of the wells. One example of such means is a pipette or pipetting robot. Such pipetting robots are widely employed in conjunction with devices such as plate readers, and other high-throughput applications.

### J. Pipettors

Pipetting technique affects washing and recovery performance. Use of a wide bore pipette tip allows for rapid solution introduction and removal and for enhanced recovery of levitated cells. Preferably, introduction of the pipette tip into a levitated sample should displace the volume of solution substantially equivalent to the displacement volume of the pipette tip. The volume withdrawn by the pipettor is preferred to be equivalent to the volume displaced by the insertion of the pipette tip. In an embodiment, the volume is continuously withdrawn as the pipette is inserted into the sample so that the total volume of sample well remains substantially constant during introduction of the pipette tip into the sample solution.

The pipetting techniques disclosed herein are applicable to single and multichannel pipettors including 4, 6, 8, 12, 16, 24, 48, 96 and 384 channel pipettors. Examples of manual multichannel pipettors include Eppendorf Research Plus pipettors, Gilson PIPETMAN L or G Multichannel pipettors, Rainin Pipet-Lite XLS+ pipettors, Thermo Fisher Finnpipette F2/F3 pipettors, Integra Viaflow/Voyager pippetors, and Sartorius Tacta Multichannel pipettors. Examples of electronic multichannel pipettors include Eppendorf Xplorer/Xplorer plus pipettors, Gilson PIPETMAN M pipettors, Rainin E4 XLS+ pipettors, and Thermo Fisher El-ClipTip pipettors. For high-throughput applications, the pipettors are preferably incorporated in to automated pipetting robots such as or similar to Hamilton Microlab STAR/STARlet/VANTAGE robots, Analytik Jena CyBio FeliX robots, Integra Assist/Assist Plus, Tecan Freedom EVO/Fluent robots, Opentrs OT-2/OT-3 robots, Agilent Bravo Liquid Handling Platforms, Eppendorf epMotion robots, Formulatrix MANTIS+TEMPEST+F.A.S.T. robots, and Beckman Coulter Biomek Series robots. The pipettor robots can optionally be equipped with a gripper or manipulator arm to facilitate high-throughput performance of particle isolation and/or washing, and for incorporation of the recovered and/or washed samples into downstream applications such as sequencing library preparation, PCR preparation, ELISA workflows, proteomic screening, flow sorting, transfection, and cell culture. Use of multichannel pipettors in high-throughput applications with multiwell plates is preferred. The pipettor, and thus, the tips should preferably be substantially horizontally level with the plane of the multiwell plate in the levitation Array or other configurations of the stacked Magnet Array in the Magnet Holder, which should level on the deck of the pipettor robot. Alignment of the pipettor tips is preferred to be with the center of the sample wells. In an embodiment, the pipettor robot is equipped with an optical alignment system to align or verify alignment of the pipettor tip(s) with the center of the sample well(s). In applications utilizing a stacked linear array or linear Halbach array, horizontal movement of the pipette tip(s) along the line of levitated particles during removal of particles from the sample solution can enhance recovery of the levitated particles of interest.

### K. Sample Volumes

Sample volumes processed by the magnetic arrays of the present invention can range from about 10 μL to about 400 mL with additional operable ranges in high-throughput applications from about 10 μL to about 1 mL, about 15 uL to about 900 uL, about 20 uL to about 800 uL, about 25 uL to about 500 μL, and about 25 uL to about 100 uL. Examples of high-throughput sample volumes include about 25 uL, about 30, 40, 50, 60, 70, 80, 90, and 100 uL. Large volume applications of about 1 mL to about 30 mL can be singularly processed (or in a large array fashion) using high magnetic field strength permanent magnets such as NdFeB (N52) magnets, rare-earth-free magnets (e.g. Fe—Ni or Fe—Co alloys) and electromagnets in an LV array as configured in in FIGS. 25 and 26A. Volumes of about 30 mL to about 400 mL, including volumes of about 50 mL to about 500 mL, such as the volume of a Leukopak, can be processed in the methods of the present invention using REBCO high-temperature superconducting magnets with a maximum field strength of about 32 T, an Nb3Sn low-temperature superconducting magnets with a maximum field strength of about 23 T, a NbTi low-temperature superconducting magnet with a maximum field strength of about 12 T, or a higher field strength cooled resistive Bitter magnets in circular configurations with maximum field strengths of about 45 T.

### L. Magnetic Beads

Magnetic beads are widely used for the depletion of specific cells or organelles or types of debris, such as myelin, from complex biological samples through magnetic separation. These beads can be coated with antibodies, streptavidin/avidin, oligonucleotides, lectins, small molecules including drugs, protein ligands such as those acting as receptor or substrate mimics, affinity tags such as recombinant tagged proteins, and aptamers. When mixed with a particle sample, the beads attach to the intended targets. When the sample well containing the sample and magnetic beads are placed into the magnetic field of the LV Array or other configurations of the stacked Magnet Array in the Magnet Holder, a magnetic field is applied drawing the bead-bound targets to the side of the well, allowing unbound components to be removed. This method enables the efficient, rapid, and gentle removal of specific populations and debris-such as T cells, red blood cells, mitochondria, exosomes, and myelin-without affecting the remaining sample. Following removal of the levitated particles and removal of the well from the magnet array, the magnetic particles and their bound substrate can be recovered from the well, either manually or by robot.

### M. Paramagnetic Medium

Samples processed by magnetic facilitated concentration by the present invention, e.g., Methods 6 and 6A, will typically have an added paramagnetic component or an added diamagnetic component. In accordance with the method of the present invention, a substance containing particles of interest is combined with a paramagnetic medium to create a sample fluid or sample suspension. The paramagnetic medium comprises a paramagnetic material and a solvent. In accordance with a preferred embodiment, the paramagnetic medium is biocompatible, i.e. capable of being mixed with live cells and without impacting the viability of the cells or impacting cellular behavior, e.g. impacting gene expression. The paramagnetic material may be selected from the group comprising gadolinium, titanium, vanadium, dysprosium, chromium, manganese, iron, nickel, gallium, including ions thereof and combinations thereof. In accordance with an embodiment the paramagnetic material is selected from the group comprising titanium (III) ion, gadolinium (III) ion, vanadium (I) ion, nickel (II) ion, chromium (III) ion, vanadium (III) ion, dysprosium (III) ion, cobalt (II) ion, and gallium (III) ion. In accordance with a preferred embodiment, the paramagnetic material comprises a chelated compound. In accordance with a preferred embodiment, the paramagnetic material comprises a gadolinium chelate, e.g., gadobutrol, a dysprosium chelate, or a manganese chelate. In accordance with an embodiment, the paramagnetic medium comprises a paramagnetic material, salts, and other additives that function to maintain cellular integrity. In an embodiment of the invention the paramagnetic material may be [Aliq]₂ [MnCl₄], [Aliq]₃ [GdCl₆], [Aliq]₃ [HoCl₆], [Aliq]₃ [HoBr₆], [BMIM]₃ [HoCl₆], [BMIM] [FeCl₄]. [BMIM]₂ [MnCl₄], [BMIM]₃ [DyCl₆], BDMIM]₃ [DyCl₆], [AlaC1] [FeCl₄], [AlaC1]₂ [MnCl₄], [AlaC1]₃ [GdCl₆], [AlaC1]₃ [HoCl₆], [AlaC1]₃ [DyCl₆], [GlyC2] [FeCl₄] as described in U.S. patent application Ser. No. 14/407,736 which is incorporated herein by reference.

In accordance with an embodiment, the paramagnetic material may be present in the paramagnetic medium at a concentration of at least about 10 mM, 20 mM, 30 mM, 40 mM, 50 mM, 60 mM, 70 mM, 80 mM, 90 mM, 100 mM, 120 mM, 150 mM, 200 mM, 250 mM, 300 mM, 500 mM, or 1 M. In accordance with an embodiment, the paramagnetic material may be present in the paramagnetic medium at a concentration of about 10 mM to about 50 mM, about 25 mM to about 75 mM, about 50 mM to about 100 mM, about 100 mM to about 150 mM, about 150 mM to about 200 mM, about 200 mM to about 250 mM, about 250 mM to about 300 mM, about 300 mM to about 500 mM, or about 500 mM to about 1 M.

In accordance with an embodiment, the paramagnetic material comprises gadolinium, e.g. gadobutrol, and is present in the paramagnetic medium at a concentration of at least about 10 mM, 20 mM, 30 mM, 40 mM, 50 mM, 60 mM, 70 mM, 80 mM, 90 mM, or 100 mM. In accordance with an embodiment, the paramagnetic material comprises gadolinium, e.g. gadobutrol, and is present in the paramagnetic medium at a concentration of about 10 mM to about 50 mM, about 25 mM to about 75 mM, or about 50 mM to about 100 mM.

### N. Isolation and Concentration Methods

A population of interest, for example organic or inorganic particles, may be concentrated by the methods of the present invention. The particles may be biological entities such as cells, cell fragments, organelles (e.g., cell nuclei and chloroplasts), clusters, tissue, tissue components, microorganisms including bacteria, fungi (yeasts and molds), viruses, protozoa, and algae and fragments, organelles, clusters, and other components thereof. Particles can be macromolecules, complexes, chelates, conjugates, crystals, amorphous solids, gels, coagulates, and the like. DNA, RNA, proteins, are concentratable under methods of the present invention. Beads, shells, nanoparticles, laminates, and precipitates and coprecipitates may likewise be concentrated. Numerous applications require the isolation of particles, including applications requiring the separation of like particles from other particles, identification of particles, and the treatment or otherwise manipulation of particles. Such applications include, but are not limited to, separating live and dead cells, separating cell nuclei from live and dead cells and nuclear debris, isolation and/or treatment of circulating tumor cells, emulsion PCR enrichment, production of plasma such as platelet rich plasma, isolating sperm for specific traits such as gender selection, bacterial load testing, antibiotic resistance testing, identification of sepsis or blood contamination, immune cell isolation, compound screening, exosome separation, or extracellular vesicles separation. The particle isolation methods of the present invention may be utilized in any of these applications.

Particles present in a sample medium are concentrated in a particle concentrating device under conditions that substantially enrich particle concentration and substantially deplete a layer of sample medium. Sample medium with heterogeneous particle populations may be selectively enriched based on size, density, and paramagnetic heterogeneity and selective orientation of magnetic forces and processing channel flow rates. The heterogeneous population of particles may be derived from biological samples. In some cases, the biological samples are, as illustrating examples, a bodily fluid including blood, saliva, urine, sperm, plasma, serum, and stool; swabs including skin, anal, nasal and vaginal swabs or environmental swabs from a door handle; and proximal fluids including tears, lavage fluid from lungs, or interstitial tissue fluids from a breast. In some cases, the biological samples are, as illustrating examples, live and dead cells, lysed cells, circulating tumor cells, nucleic acids, nucleotides, amino acids, peptides, proteins, antigens, antibodies, or immune cells (e.g., white blood cells. T cells, phagocytic cells). In some cases, the biological samples are, as illustrating examples, a biomolecule, cell, protein, lipid, carbohydrate, microorganism, virus, virion, or bacteria.

Level of concentration of the particle enriched fraction over particle concentration in the sample medium is at least 30%, preferably 40%, 50%, 60%, 70%, 80%, 90%, 95%, or 99%. In an embodiment, the particle depleted fraction is substantially free of particles.

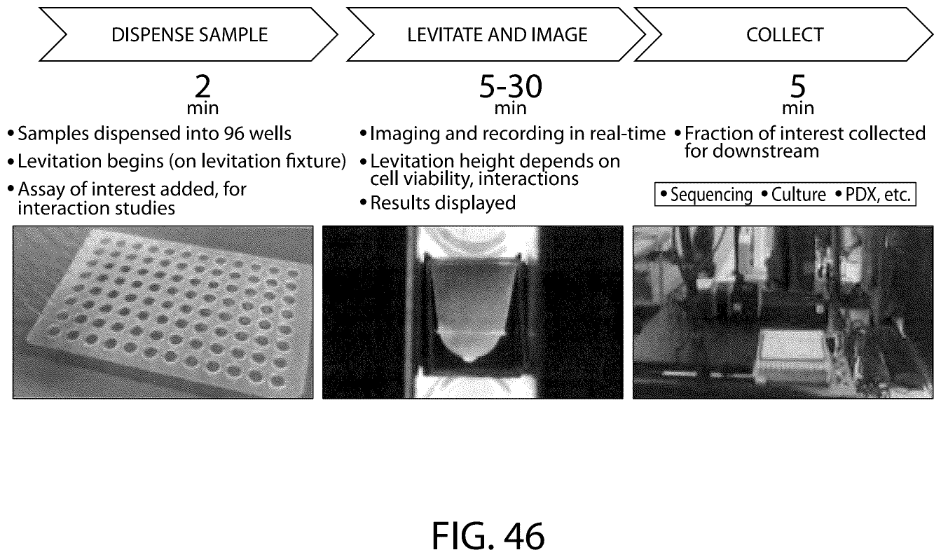

FIG. 46 shows a typical workflow for a device of the present invention. Samples are dispensed into wells of the multi-well plate (about 2 minutes), and levitation is commenced. Levitation is allowed to proceed until the components reach their levitation equilibrium positions, typically 5-30 minutes, during which time the wells are imaged and recorded in real time. The fraction. of interest (e.g., a small volume containing live cells) is then collected as described below in a process that typically takes about 5 minutes or less.

In some embodiments, a population of interest, e.g., particles of interest, is separated from one or more contaminating species by the methods described herein. In some embodiments, the contaminating species is selected from one or more of a dissolved or suspended compound (e.g., a dye or dyes, antibodies, etc.); cellular debris; small particles (e.g., micro- or nano-beads); and dead cells.

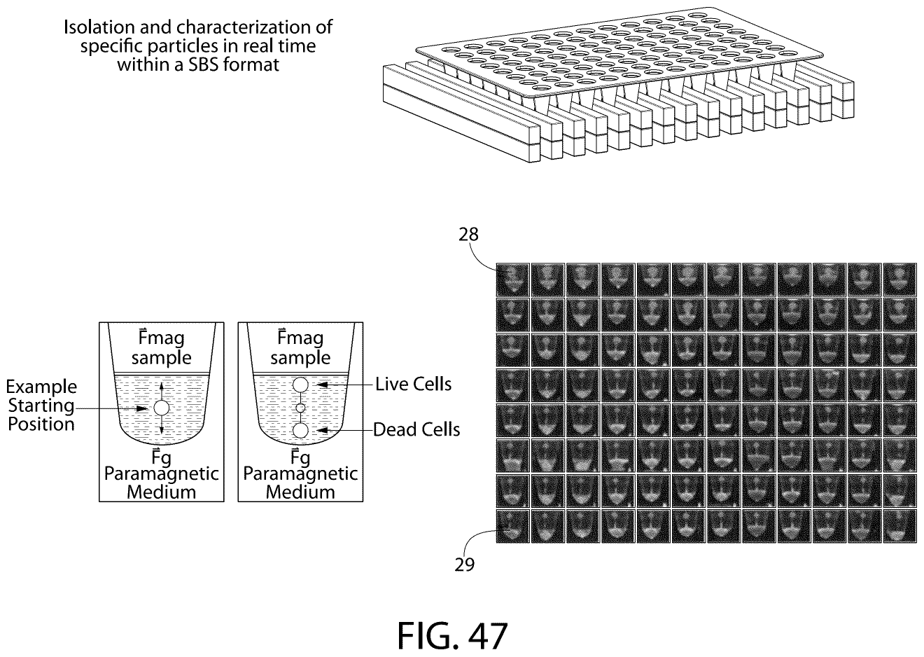

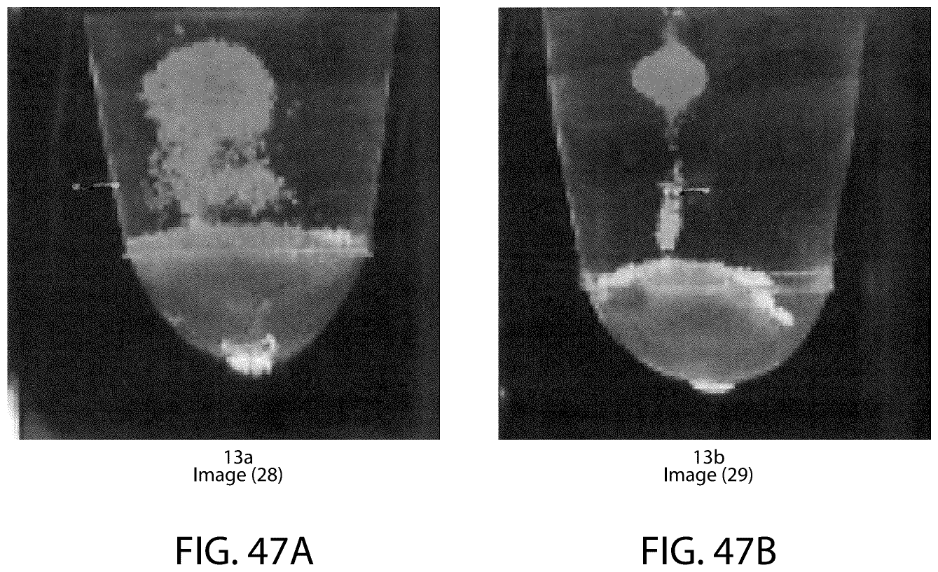

In one embodiment, live cells can be isolated from a sample comprising said live cells and one or more contaminating species. In one such embodiment, the sample and a paramagnetic medium comprising a paramagnetic compound or ferrofluid are loaded into a well of a system as described herein (e.g., System 1) to form a sample fluid or sample suspension in the well. The sample fluid or sample suspension is then subjected to the magnetic force from at least one magnet from the magnet array, and the components of the fluid or suspension are allowed to levitate to their equilibrium positions. FIG. 47 illustrates the concentration of particles (e.g., live and dead cells) utilizing a device of the present invention. The lower left panel shows a representation of the starting position and equilibrium levitation positions of live and dead cells in a well of the multi-well plate. The image on the lower right is a composite of images taken of all 96 cells used to separate green and red colored beads of different density. The top left image (28) is first in time and shows a diffuse pattern, which over time sharpens to show clear separation of the beads, as shown in bottom left image (29). These images are shown in detail in FIGS. 47a and 47b, respectively.

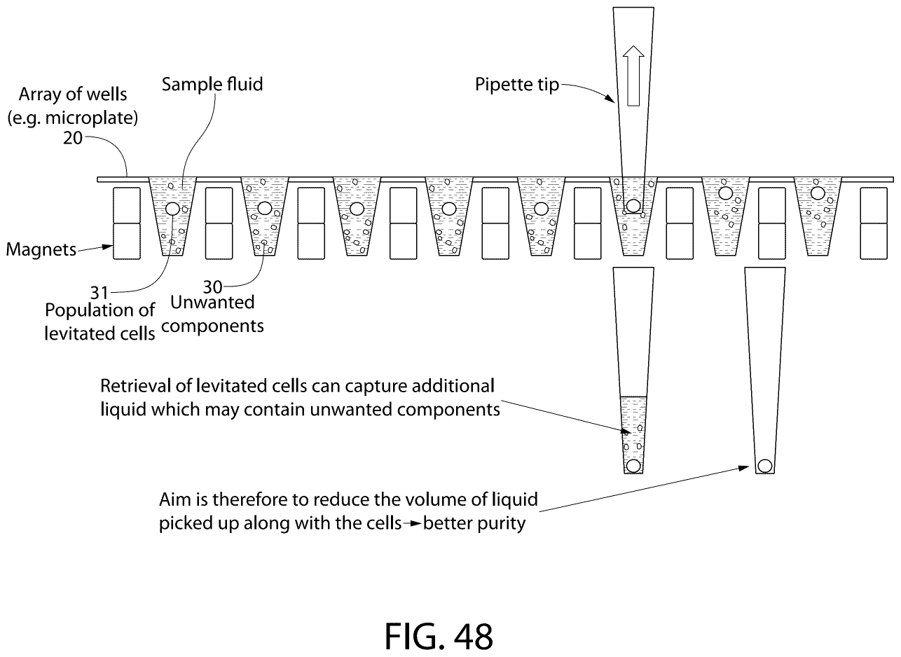

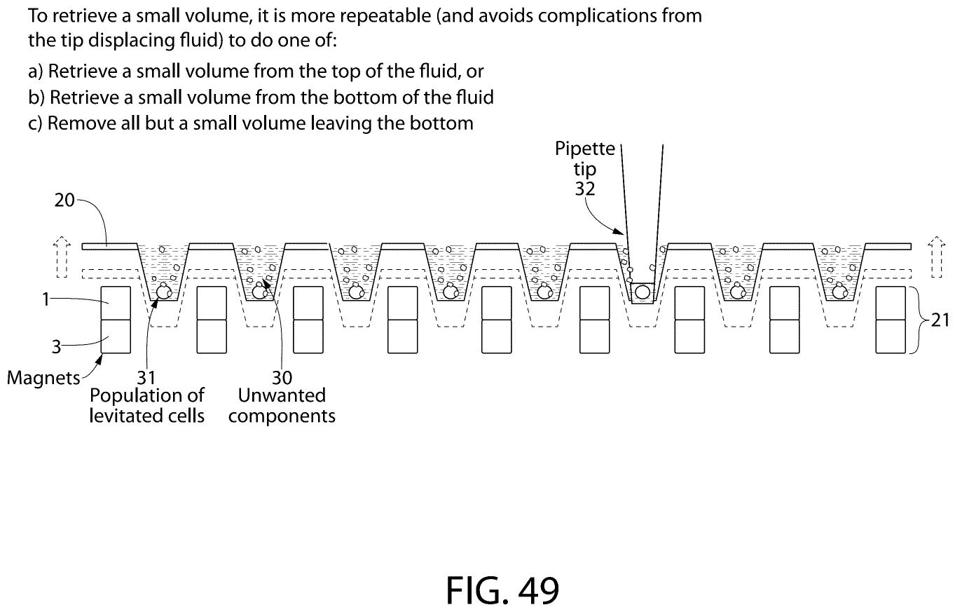

Collection of a particles can be achieved by any of several modalities. FIG. 48 depicts the separation of levitated cells (31) from unwanted components (30) using a pipette tip (32), for example a pipette tip of a pipetting robot. Retrieval of levitated cells (31) can also capture additional liquid that may contain unwanted components (30). Accordingly, it is advantageous to minimize the volume of liquid collected with the cells. FIG. 49 depicts the retrieval of cells by a) moving the multi-well plate (20) vertically upward relative to the magnet array (21) while maintaining the separated live cells (31) in place with the magnetic field, thereby causing the separated live cells (31) to migrate to the bottom of the well; and then either: i) removing the liquid above the separated live cells with the pipette tip (32), leaving the separated live cells (31) in a reduced volume of fluid at the bottom of the well; or ii) removing the separated live cells (31) from the bottom of the well with the pipette tip (32). The arrow inside the pipette tip (32) in FIG. 49 indicates movement of liquid, and the dashed arrow outside the pipette tip (32) in FIG. 49 indicates movement of the multi-well plate (20).

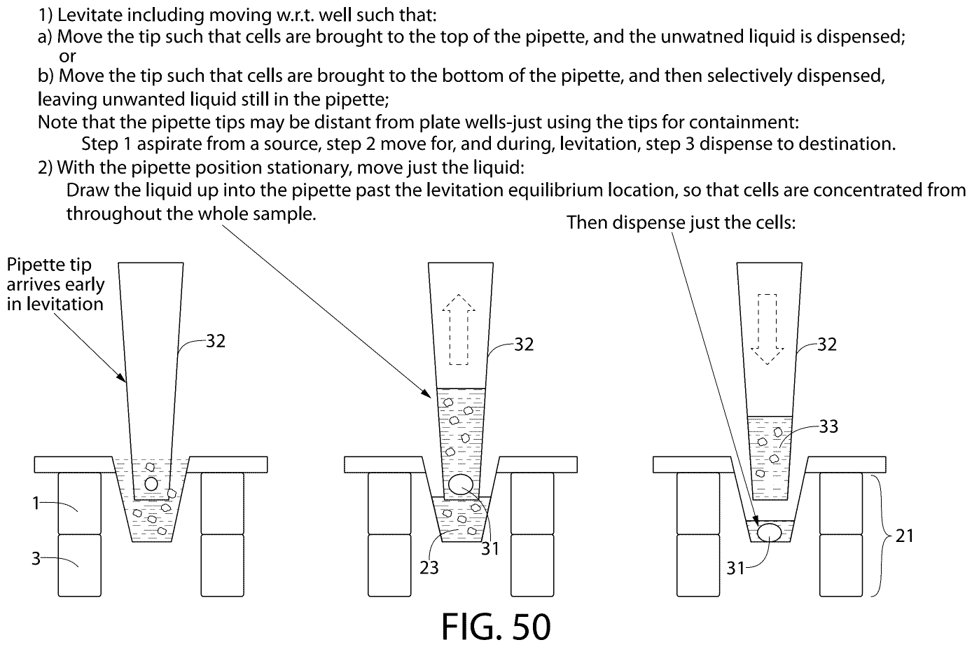

FIG. 50 depicts the retrieval of cells by either 1) moving the pipette tip (32) a vertical direction while maintaining the separated live cells (31) in place with the magnetic field, to bring the cells to the top of the pipette tip (32); and 2) dispensing the unwanted liquid below the cells (33) while retaining the separated live cells (31) in the pipette tip (32); or 1) moving the pipette tip (32) in a vertical direction while maintaining the separated live cells (33) in place with the magnetic field, to bring the separated live cells (33) to the bottom of the pipette tip (32); and 2) selectively dispensing the separated live cells (33), leaving unwanted liquid (33) in the pipette tip. In a further embodiment, retrieval of cells can be accomplished by a) early in the levitation, inserting the pipette tip (32) into the sample past the levitation position of the separated live cells (31); b) drawing the liquid up into the pipette tip (32) while maintaining the separated live cells (31) in place with the magnetic field; and c) dispensing just the separated live cells (31).

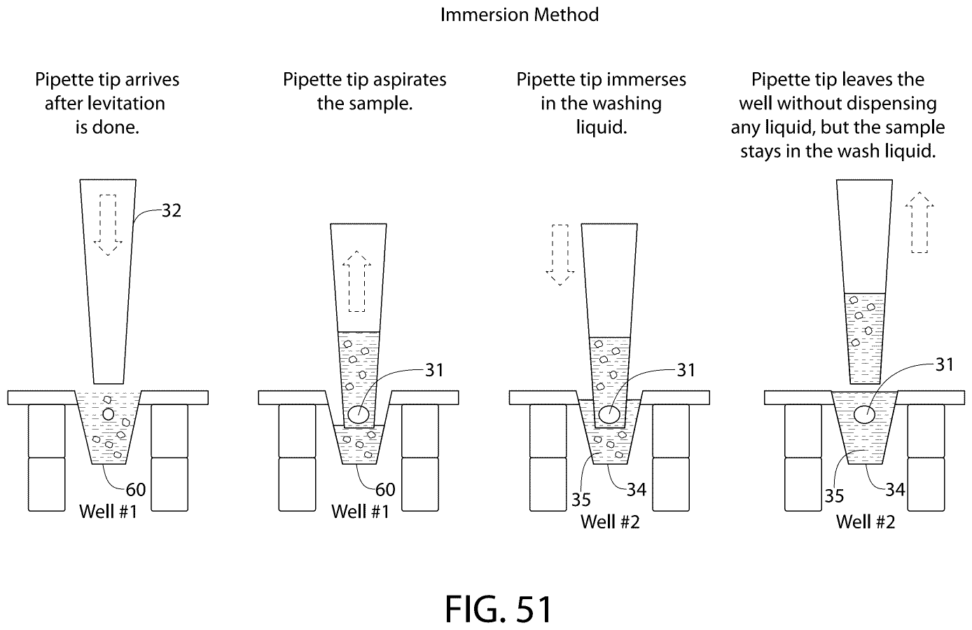

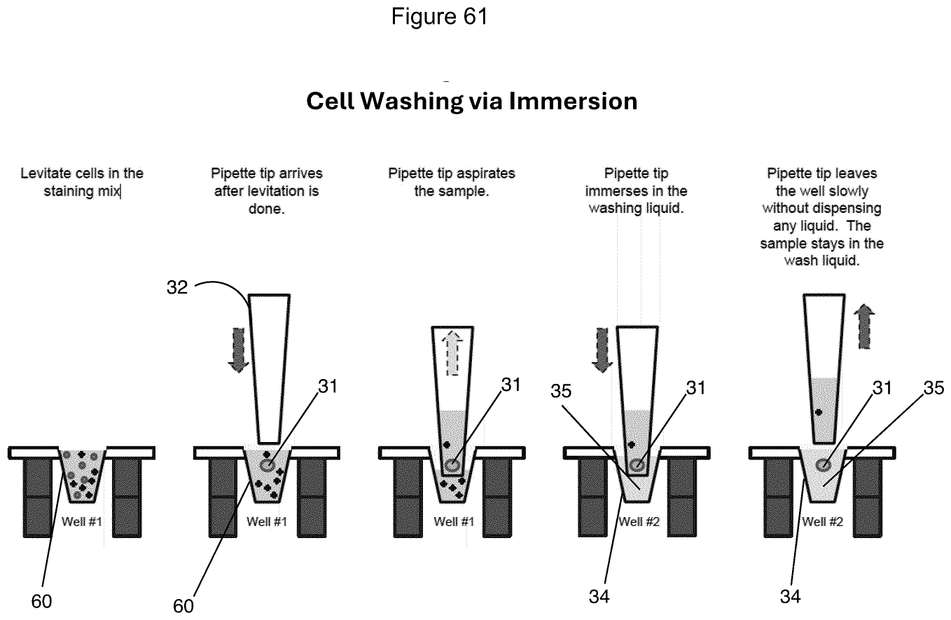

FIGS. 51 and 61 depict the retrieval of cells by an immersion method according to the following steps: a) levitating the cells, optionally in a staining mix; b) after achieving levitation equilibrium, aspirating the liquid into the pipette tip (32) until the separated live cells (31) are aspirated; b) withdrawing the pipette tip (32) from the well (60) and immersing it into a second well (34) in the plate or a separate plate that contains a washing liquid (35); c) aspirating the separated live cells (31) into the washing liquid (35) while retaining the liquid in the pipette tip (32); i.e., slowly removing the pipette tip and the liquid therein, while the field maintains the position of the levitated cells, thus separating them from the original liquid; and d) optionally repeating steps (a)-(c).

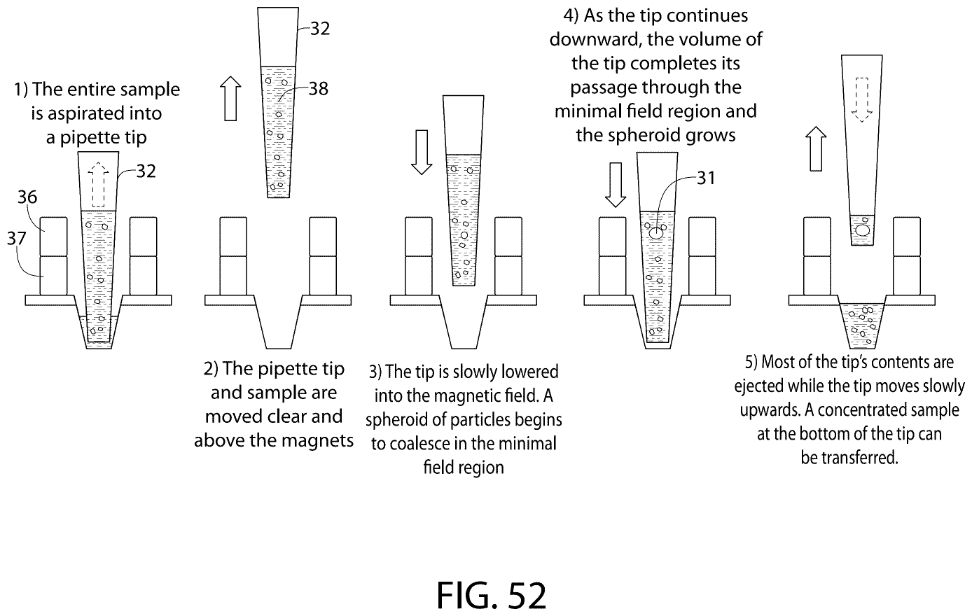

FIG. 52 depicts one mode of retrieval of cells (31) levitated by radial magnets (36) and (37). The entire sample is aspirated into the pipette tip (32). The pipette tip (32) and sample (38) are then moved vertically upwards until they are clear of, and above, radial magnets (36) and (37). The tip (32) is then slowly lowered into the magnetic field, allowing a spheroid of particles (31) to begin to coalesce in the region of minimal field. As the tip (32) continues to move downward, the volume in the tip (32) completes passage through the region of minimal field and the spheroid (31) grows. Then most of the contents of tip (32) are ejected while the tip (32) is moved slowly upwards, leaving a concentrated sample (31) at the bottom of the tip (32) that can be transferred.

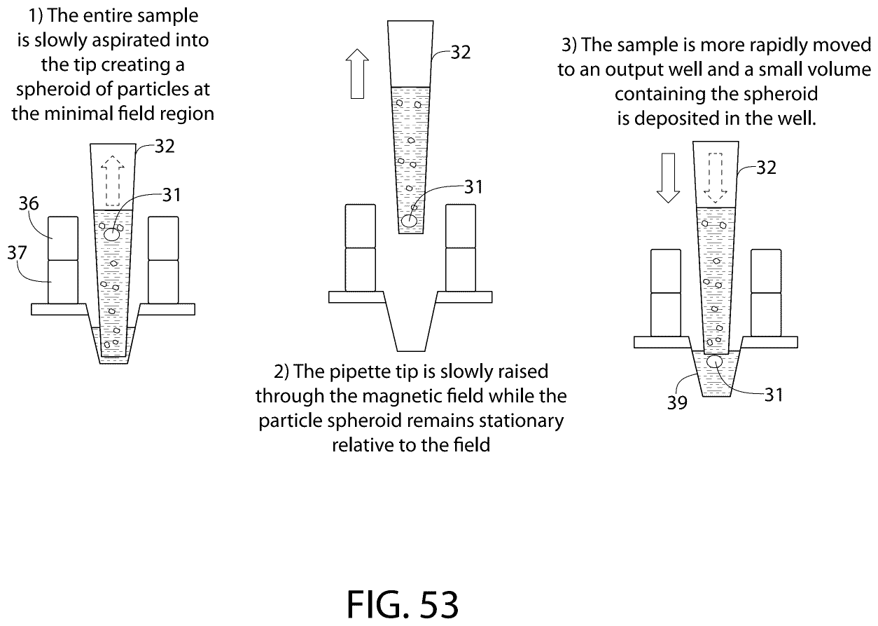

FIG. 53 depicts another mode of retrieval of cells levitated by radial magnets (36). and (37). The entire sample is slowly aspirated into the pipette tip (32) creating a spheroid of particles, e.g., levitated live cells, (31) at the region of minimal field. The pipette tip (32) is then moved vertically upwards through the magnetic field while the particle spheroid (31) remains stationary relative to the field. The sample is then rapidly moved to an output well (39) and a small volume containing the spheroid (31) is deposited in the output well (39). The methods described herein produce exceptional recovery and purity of live cell fractions. The enriched recovered sample fraction comprises at least about 60%, at least about 70%, at least about 80% or at least about 90% live cells and the yield of live cells in the enriched recovered sample fraction is at least about 50%, at least about 60%, at least about 70%, or at least about 75% of the total live cell composition of the sample. For embodiments in which nuclei are isolated, the smaller nuclei will levitate at a lower level in the well than cells (potentially due to porosity and density differences between intact viable cells and intact nuclei.) For recovery of nuclei with enhanced purity, the nuclei sample is pipetted into a levitation well and the sample nuclei are allowed to reach or closely approach their magnetic levitation equilibrium height. To achieve high purity, an upper layer of the sample is first withdrawn leaving the enriched nuclei in the well, following which the nuclei are withdrawn and deposited in an output well.

In some embodiments wherein stained cells are levitated, the cells can first be stained and after a period of time, for example one hour, the cells can then be levitated and separated. In some other embodiments wherein stained cells are levitated, the cells can be mixed with the stain(s) and immediately placed into the levitation well and levitated—i.e., staining and levitating the cells at the same time. In some further embodiments, one or more of the staining and levitating steps can be performed at room temperature, or at reduced temperature, for example at 4° C.

Isolation of particles, e.g., live cells, as described herein is rapidly accomplished without subjecting the particles to the stresses associated with other washing and/or separation techniques such as FACS or wash/centrifugation. The total time for separation can range from about 1 minute to 2 hours, for example from about 12 minutes to about 40 minutes. Cells within a solution containing multiple types of particles may be deemed “separated” if, after processing the solution, the ratio of the cells of interest to the concentration of other types of particles is increased, or if the ratio of the concentration of the cells to the concentration of other types particles is increased by at least about 10%, 50%, 100%, 200%, 300%, 400%, 500%, 600%, 700%, 800%, 900%, or 1000%, or if the concentration of non-live cell particles in the solution is decreased by at least about 20%, 15%, 10%, 8%, 6%, 5%, 4%, 3%, 2%, 19%, or 0.5%. In a preferred embodiment, the integrity of isolated cells enriched collected portion of the sample is greater than 30% of the integrity of similar cells isolated by a method comprising centrifugation. Preferably the integrity of isolated cells in the enriched collected portion of the sample is at least 35%, 40%, 45%, 50%, 55%, 60%, 65%, 70%, 75%, 80%, 85%, 90%, 95%, 97%, or 99% of the integrity of similar cells isolated by a method comprising centrifugation. The cell types separated by the above method can include human cells, non-human animal cells, plant cells, eukaryotic cells (for example, but not limited to, immune cells, endothelial cells, yeasts and T-cells). Cells isolated according to the methods of this disclosure can be directly obtained from an organism, or from propagated or cultured cells. In a particular embodiment of the rapid, high capacity separation method, live cells are separated from dead cells. In another embodiment of the method, cells are separated from cell fragments, and/or sample debris.

The methods described herein provide information not easily obtainable by other means. For example, the methods of the present disclosure can be used to obtain information from within viable cell populations. In one embodiment, the cells can be levitated in the presence of a drug in accordance with the present disclosure, and information can be obtained regarding the interaction of the drug and the cell population. In further embodiments, information can be obtained regarding interactions between cells, e.g. immune cells and/or macrophages attacking tumor cells, without the concern of whether cells adhered to the plate and in solution exhibit differences in behavior.

### Example 1 Isolation of Nuclei from Flash-Frozen Lung and Brain Tissue

Nuclei were isolated from flash-frozen lung and brain tissue by incubating with Nuclei Lysis Buffer (ThermoFisher) for 30 minutes on ice. Nuclei were then washed with PBS containing 0.1% BSA two times before resuspending in Nuclei Storage Buffer (comprising 1M Sucrose). It has been discovered in accordance with the present invention that the use of sucrose in the Nuclei Storage Buffer is advantageous in promoting storage of the nuclei. Nuclei were prepared for separation by resuspending 3e5 nuclei in Levitation Buffer containing 100 mM Levitation Agent. A sample was set aside for comparison after separation. Nuclei were separated in a separator device as described herein (LeviCell, available from LevitasBio Inc., Menlo Park, CA) using a 30 minute equilibration period. The outputs were stained with PI and imaged using an Echo microscope.

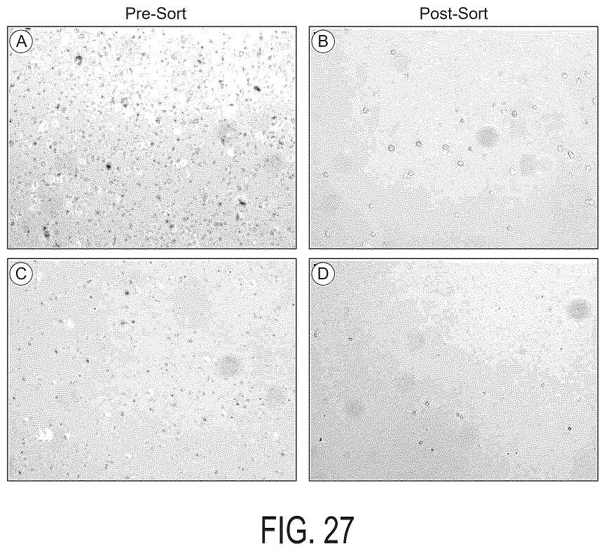

The nuclei stain brightly with PI while cellular debris do not. Images of the stained inputs and outputs from the LeviCell show a significant reduction in the amount of unstained debris for both tissue samples. The lung tissue produced more debris than the brain tissue (FIGS. 27A and C) but both samples show marked reduction in debris after sorting with the LeviCell (FIGS. 27B and D).

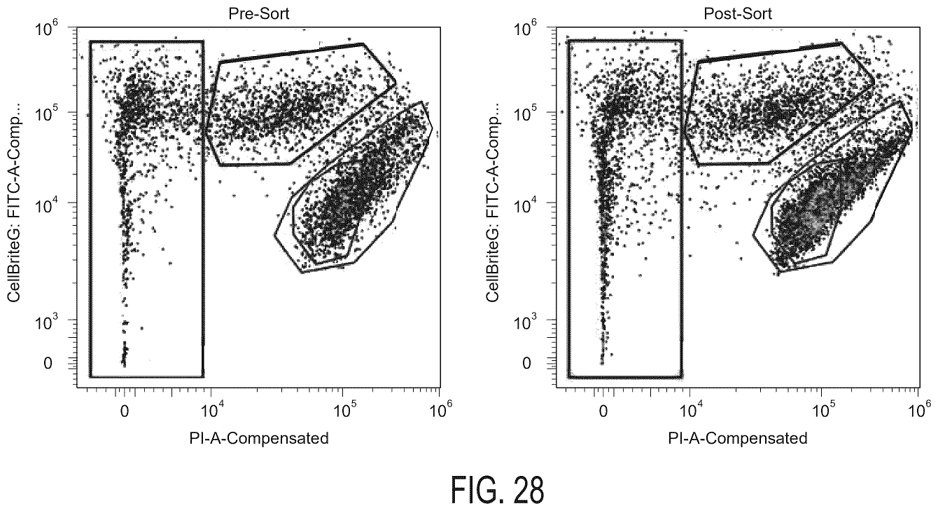

Flow cytometry analysis of unsorted (FIG. 28A) and sorted (FIG. 28B) nuclei derived from Jurkat cells. Samples were stained with PI and CellBrite Fix Green for 15 minutes at room temperature before analyzing on Sony SH800S sorter. Both the debris and dead cell populations are reduced after sorting and the nuclei population is enriched (50.81% to 63.57%). Additionally the singlet nuclei population is enriched from 28.28% to 43.98%.

### Example 2—Isolation of Nuclei from Jurkat Cells

Jurkat cells (ATCC #TIB-152) were aged for several days without media change to induce cellular senescence and apoptosis. These cells were collected and counted using AO/PI on a Nexcelom Spectrum cellometer. A portion of these cells were retained for total RNA input control. Nuclei from 8 million cells at approximately 50% viability were extracted using nuclei EZ lysis buffer (sigma) containing RNAse Out RNAse inhibitor (Thermo). Nuclei were pelleted and resuspended in levitation buffer containing 100 mM Gadolinium, 1×PBS, 1% BSA, and RNAse Out to 0.2U/ul—this sample is now called the input. 1 million extracted nuclei were levitated for 30′ in a LeviCell system. Nuclei were collected from the bottom channel of the Levicell cartridge, and in parallel with nuclei extracted in the earlier step (input), and whole cells retained from the initial sample, processed for total RNA using RNEasy prep mini kit (Qiagen). Total RNA was normalized and 10 ng of total RNA was used to make cDNA using Primescript RT with gDNA eraser kit (Takara). Predesigned and validated primetime qPCR probe assays (IDT) targeting 18S, SCARNA5 and SNHG6 were multiplexed in 10 ul reaction volumes containing 5 ul 2×Primetime gene expression master mix (IDT) and 200 pg equivalent of cDNA. Triplicate reactions were amplified on a Quantstudio5 (Thermo) instrument. Amplification data were analyzed using the ΔΔCT method and are reported as relative quantities normalized to 18S endogenous control and compared to whole cell RNA.

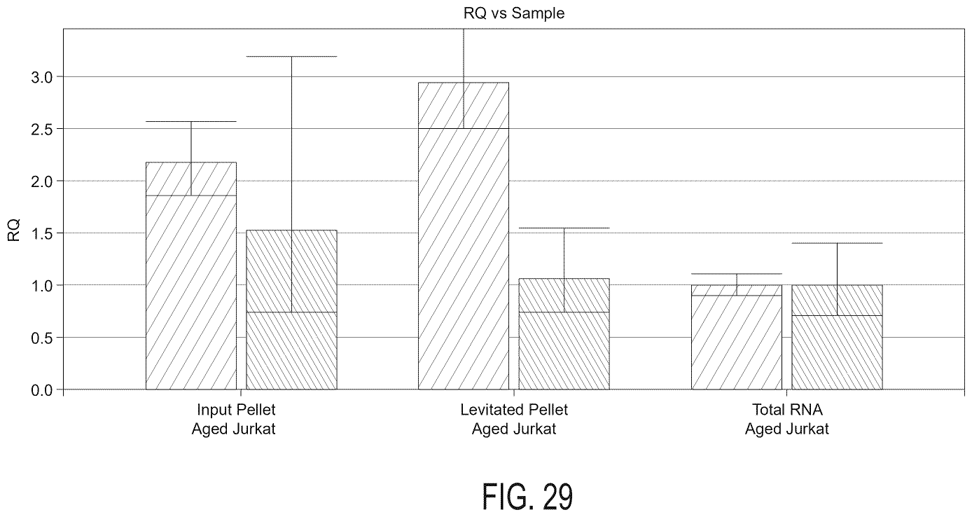

Results: As shown in FIG. 29, nuclei that were processed on the LeviCell system were more highly enriched for nuclear specific target (SCARNA5) and had less contaminating cytoplasmic target (SNHG6) compared to input nuclei. Nuclei processed on the LeviCell system exhibited nearly a 3-fold enrichment for nuclear specific signals compared to whole cells while exhibiting no enrichment of cytoplasmic target. Levitated samples also exhibited 50% greater enrichment of nuclear specific signal compared to input nuclei. In comparison, input nuclei displayed a lesser enrichment of nuclei dependent target signal and greater amounts of contaminating cytoplasmic RNAs when compared to whole cell RNAs. These data show that the levitation of nuclei on the LeviCell system enables for enrichment of nuclei and nuclear specific RNAs when compared to nuclei extracted by routine methods.

### Example 3—Effect of WGA on RNA Yield of Isolated Cell Nuclei

Nuclei were isolated from human brain by incubating in a nuclei isolation buffer, and the same buffer containing either 0.1 mg/mL WGA or 0.3 mg/mL WGA. The resulting nuclei were resuspended in the same buffer, and amount of RNA determined for each preparation. FIG. 30 shows the data for total yield of RNA in nuclei isolation buffer, with and without WGA, and ng RNA/nuclei for the nuclei resuspended in isolation buffer, with and without WGA. It can be seen that the addition of WGA, particularly at 0.1 mg/mL, resulted in an increased yield of RNA retained when performing bulk RNA extraction.

### Example 4—Isolation of Human Brain Nuclei with LeviCell Using 3 Micron Streptavidin Coated Polystyrene Beads

The LeviCell was used to separate human brain nuclei from a preparation containing the nuclei and both large and small cellular debris. Human brain nuclei were isolated and prestained with propidium iodide in a wash buffer, and then resuspended in PBS/BSA/propidium iodide. 3-3.4 micron streptavidin coated polystyrene beads (SVP-30-5, Spherotech, Inc., Lake Forest IL) were then added to a concentration of approximately 130 beads/nuclei. Controls with no beads were also prepared. Gadolinium levitation buffer was added to the samples to provide 150 mM gadolinium, and the samples were loaded on the LeviCell.

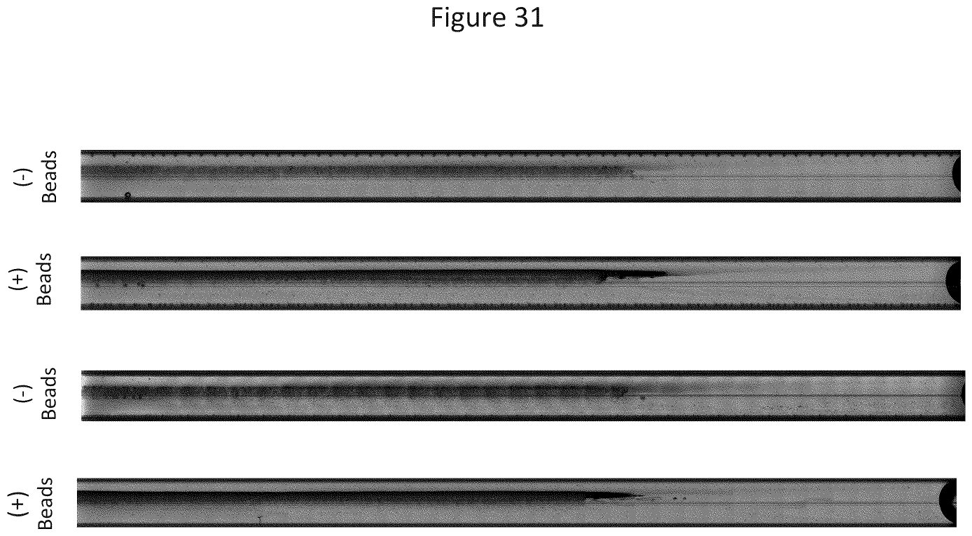

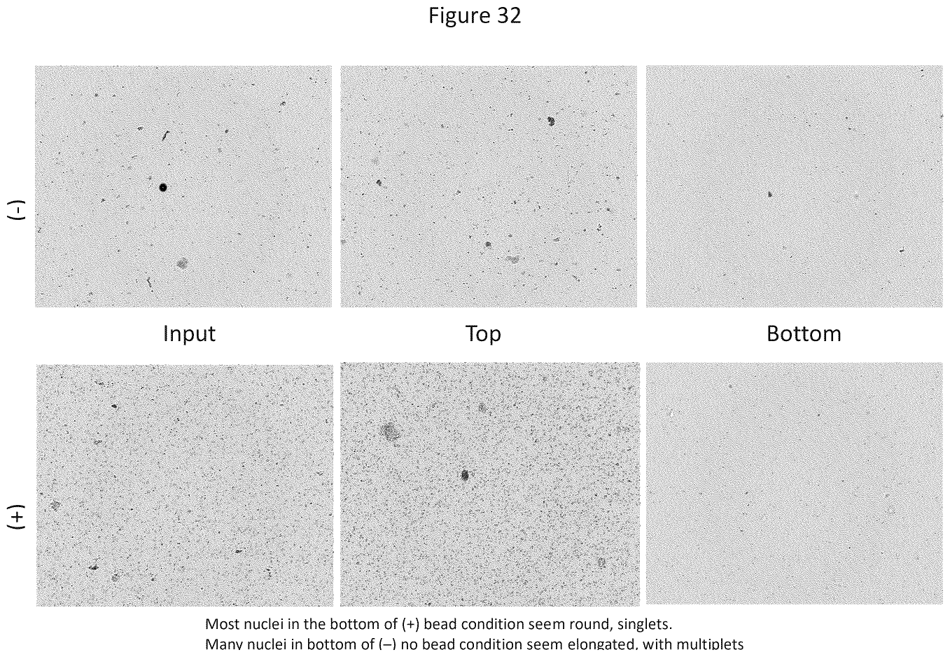

The results are shown in FIG. 31, which shows images of the flow cell during the separation. It can be seen that the two samples with beads (“(+) Beads”) have a dense band in the upper lane composed of the beads and mostly non-nuclei debris. In contrast, the two control samples with no beads (“(−) Beads”) display significantly less separation of nuclei. FIG. 32, which shows images from a cell counting system of the top and bottom fractions from the Levicell, also indicates that the nuclei obtained in the presence of the beads (bottom right panel) appear to be rounder and in better condition, and mostly singlets. In contrast, the nuclei obtained without beads (top right panel), which appear to contain more aggregates (multiplets) and misshapen (elongated) nuclei.

These results indicate that the beads appear to be capturing the nuclei aggregates and potentially membranous debris, and that the presence of the beads in the isolation improves the appearance of the nuclei.

### Example 5—Isolation of Human Brain Nuclei with LeviCell Using 3 Micron Streptavidin Coated Polystyrene Beads and RNA Sequencing

The LeviCell was used to as described above to separate human brain nuclei, which were then subjected to RNA sequencing.

Nuclei were isolated from Human brain tissue according to the following procedure. Tissue was minced and homogenized in nuclei isolation buffer with and without WGA. The samples were filtered through 70 um sieve w/PBS/BSA/RNAseOUT, and washed (centrifugation) with 1×/PBS/BSA/RNAseOUT, and resuspended in PBS/BSA/RNAseOUT.

For Set 1, two pools were made: 1 (5rxn equivalent w/PI) and 1 (5rxn equivalent NO PI) 250 ul each, to which was added 375 ul of PBS/BSA/Pi or PBS/BSA/rnase inhibitor to 625 ul. The pools were divided into pools of 125 ul each, to which was added 80 ul bead (˜165 bead/nuclei) bead 5 minutes at room temperature or 80 ul PBS as control. 70 ul of levitation buffer was added to achieve a final concentration of 150 mM Gd. The samples were loaded on the Levicell in 150 mM, Gd 1% Pi, and the nuclei yields and purity of bottom fractions were measured.

For Set 2, to 50 ul of each preparation was added 75 ul PBS/BSA/rnase inhibitor, then 80 ul bead (˜100˜130 bead/nuclei) bead 5 minutes at room temperature, or 80 ul PBS as control. 70 ul of levitation buffer was added to achieve a final concentration of 150 mM Gd. The samples were loaded on the Levicell in 150 mM, Gd 1% Pi, and the nuclei yields and purity of bottom fractions were measured. Parallel runs were also performed without propidium iodide for use in subsequent RNA sequencing using a 3′ RNA LT kit, Target˜700 nuclei/sample.

### Results

The bottom fraction yield and purity for samples is shown in the following Table:

| | | % | Bottom | Bottom | Total |
|---|---|---|---|---|---|
| | | Nuclei- | Nuclei | output | Nuclei- |
| sample | Name | INPUT | Yield | purity | BOTTOM |
| (−) bead (+) PI | R7 | 4.1% | 51.0% | 23.4% | 4.34E+04 |
| (+) beads (+) PI | R8 | 0.8% | 80.4% | 20.7% | 3.93E+04 |
| (−) beads 1 | R9 | 2.8% | 32.5% | 14.3% | 2.43E+04 |
| (+) beads 1 | R10 | 0.2% | 67.2% | 13.1% | 2.66E+04 |
| (−) beads 2 | R11 | 2.3% | 70.5% | 13.8% | 2.36E+04 |
| (+) beads 2 | R12 | 0.4% | 18.9% | 10.1% | 2.32E+04 |
| (−) beads 3 | R13 | 14.8% | 39.0% | 24.8% | 4.70E+04 |
| (−) beads (+) WGA 1 | R14 | 16.0% | 31.5% | 31.9% | 4.63E+04 |
| (+) beads (+) WGA 1 | R15 | 3.7% | 41.3% | 30.7% | 5.46E+04 |
| (+) beads (+) WGA 2 | R16 | 4.2% | 34.1% | 17.7% | 5.45E+04 |

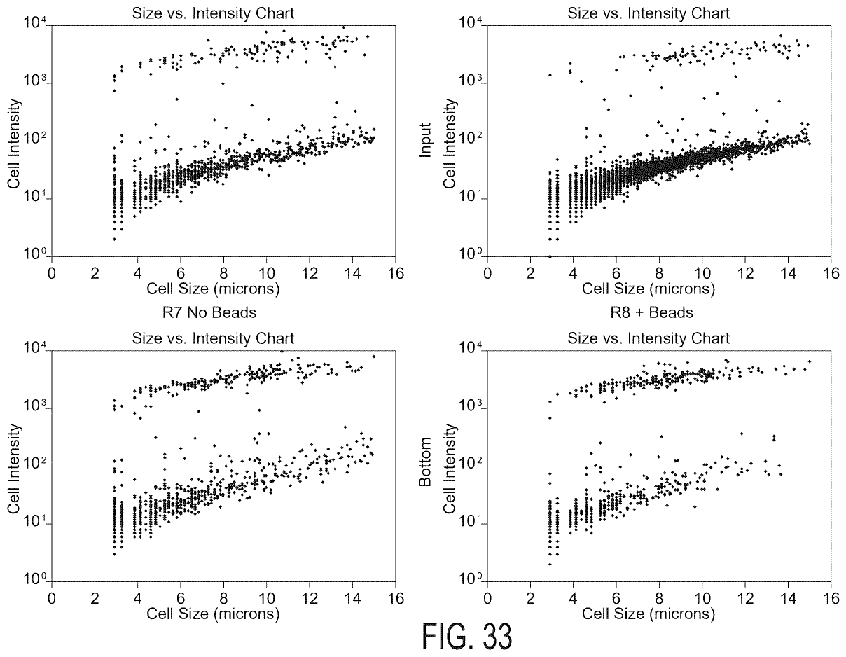

It can be seen that sample 15, which has beads and used WGA in the nuclei preparation, has both a high yield and a high purity.
FIG. 33 shows the spectrum data for the prestained nuclei with and without beads (preparations R7 and R8). The input spectra for the bead-containing sample (upper right panel) shows a dense debris field at the lower cell intensities relative to the sample lacking beads (upper left panel), which is partially due to the presence of the beads. The average fluorescent particle for the input of the beads containing sample (R8) is approximately 10 microns. This value drops in the output spectrum (lower right panel) to approximately 8 microns for the nuclei—the band at the top of the panel—and there is significantly less debris. This effect is not seen in the sample with no beads.

Results of RNA sequencing: The RNA in the samples was sequenced using a 10×3′ RNA LT kit, Target˜700 nuclei/sample. FIG. 34 shows the Seurat reordered plots for the sequenced samples. It can be seen from the percent mitochondrial chart, that samples 7 and 8, which have both WGA and the beads present, have less mitochondrial reads compared to any of the other experiments. There was also less ribosomal RNA in these preparations, which in a nuclear prep is also a cytoplasmic contaminant.

### Examples of Well-Based Embodiments Using System 1

### Example 6 Isolation of Beads

A mixture of green and red beads (fluorescent polystyrene microparticles) having different density was subjected to magnetic levitation in a 96-well plate according to the present disclosure. As shown in FIG. 47, the image on the lower right is a composite of images taken of all 96 cells used to separate the green and red colored beads. The top left image (28) is first in time and shows a diffuse pattern, which over time sharpens to show clear separation of the beads, as shown in bottom left image (29). These images are shown in detail in FIGS. 47a and 47b, respectively.

### Example 7—Cell Enrichment

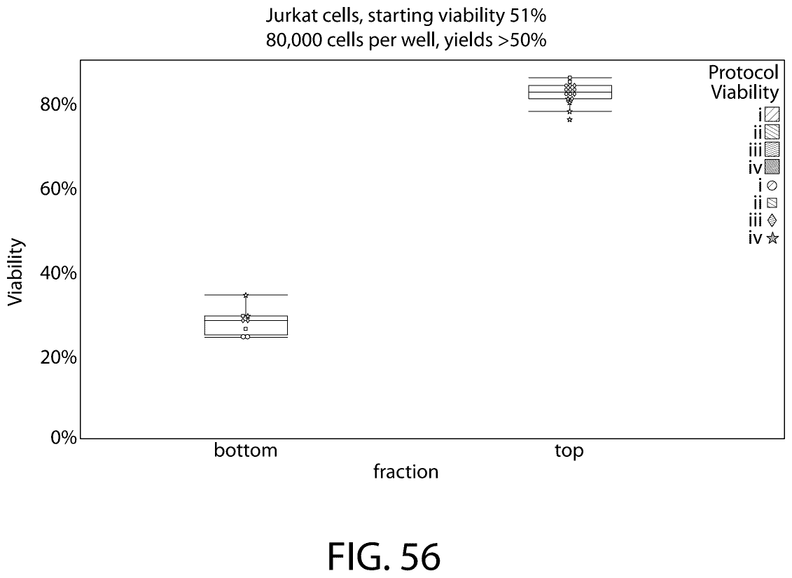

Samples containing 80,000 Jurkat cells having a cell viability of 51% were placed in wells of a multi-well plate as described herein and subjected to magnetic levitation as described herein. As shown in FIG. 56, the levitated fraction at the top of the well had a viability of over 80%, and a yield of >50%. These data show that the levitation of cells on a System according to the present disclosure enables enrichment of live cells.

### Example 8—Cell-Drug Interactions

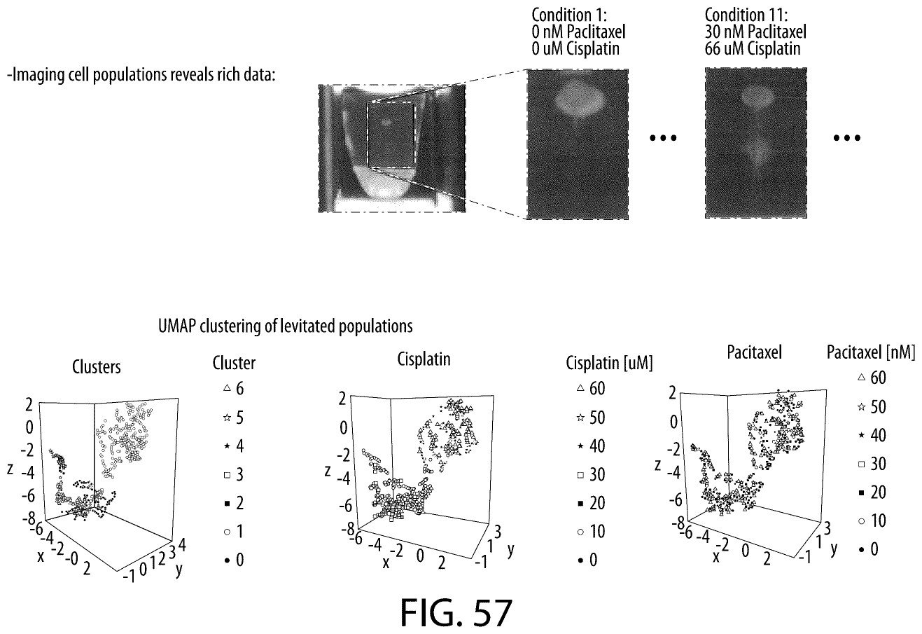

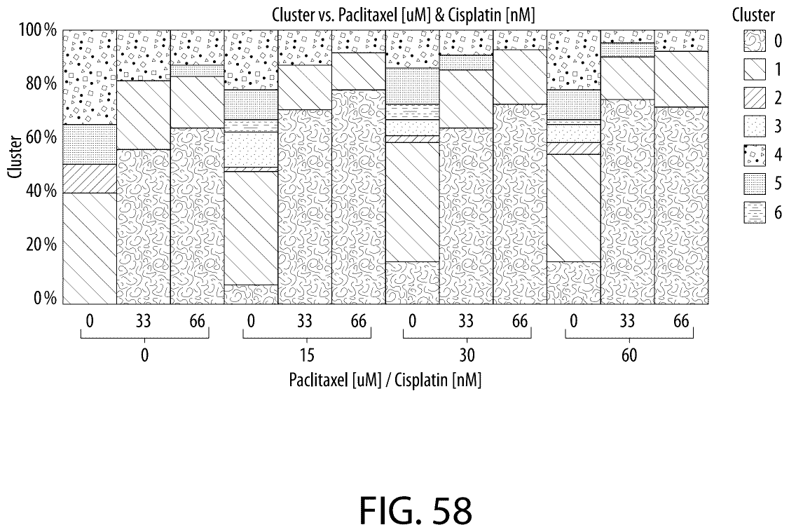

Samples were prepared containing 50,000 H358 cells in 25ul and containing 75 mM Gd levitation agent were placed in wells of a 96-well plate. The samples were levitated in the presence of 30 nM Paclitaxel and 66 μM cisplatin in accordance with the methods of the present disclosure. FIG. 57 shows UMAPs of the clustered populations that were separated by the technique. FIG. 58 shows a comparison of the clustering due to the drug treatment conditions.

### Example 9: HTS Washing—Debris and Dead Cell Removal Evaluated with FACS (Fluorescence-Activated Cell Sorting)

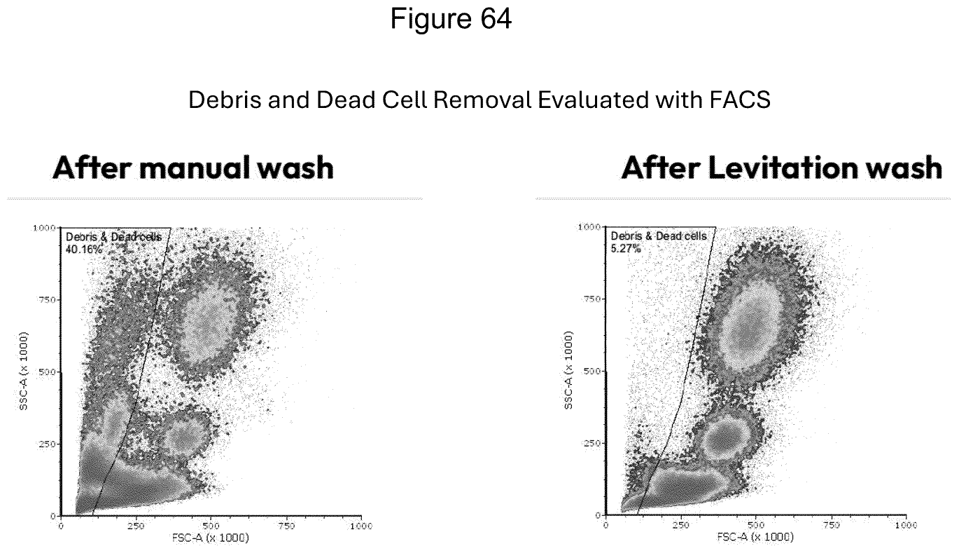

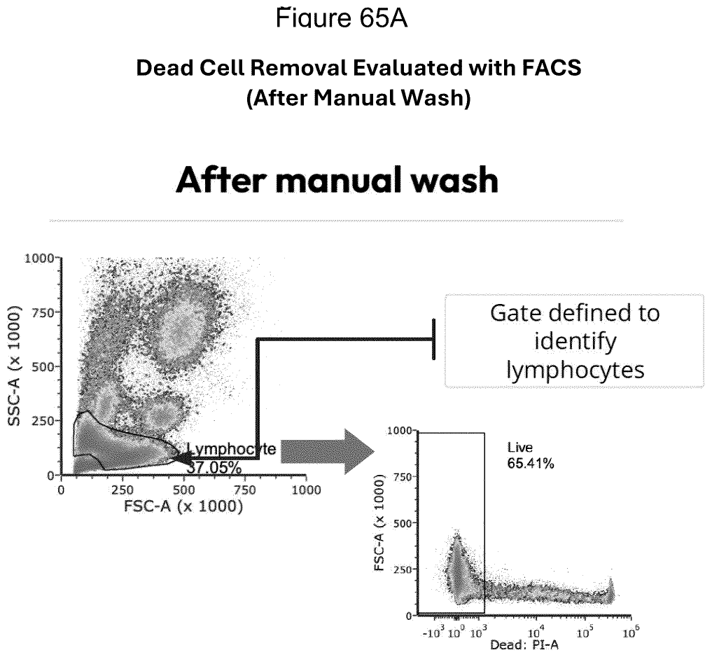

A FACS staining workflow was performed on a compromised sample of PBMCs. The FACS plots in FIG. 64 show forward and side scatter plots comparing 3 cycles of standard manual washing (with a centrifuge) to 3 cycles of Levitation washing. The results show that levitation washing effectively removes debris from the sample compared to manual washing methods, and that levitation washing can produce a high-quality sample from a low-quality input.

### Example 10: HTS Washing—Dead Cell Removal Evaluated with FACS

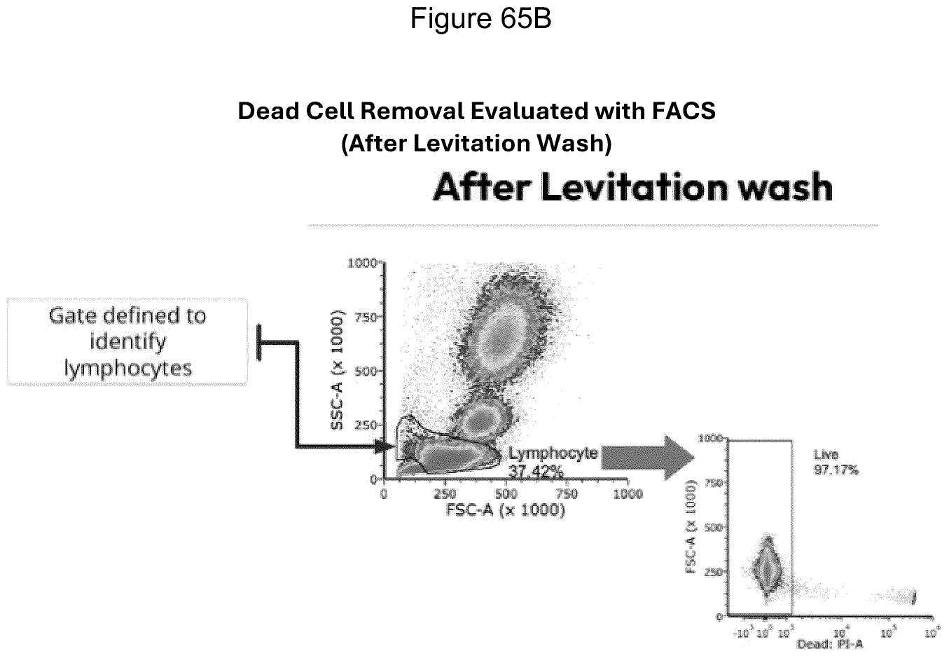

A FACS staining workflow was performed on a compromised sample of PBMCs. The FACS plots in FIGS. 65A and 65B show forward and side scatter plots that were used to create a gate to identify lymphocytes in the sample. The viable fraction of cells in the lymphocyte population was identified by the presence of PI signal indicating dead cells. The results show that levitation washing effectively removed dead cells from the input population while manual washing did not.

### Example 11: Wash Factor Measurement

An experiment was performed to measure the wash factor across 3 cycles of Levitation washing. The input condition was of a known concentration of a fluorophore. The fluorophore concentration was measured at each step of the wash cycle using a fluorimeter. The measured wash factor after on wash was 17, and after two washes was 462. The fluorophore concentration at the third step (after the third wash) was below the fluorimeter's detection range. However, the wash factor is assumed to be 4600, i.e., a 10× dilution due to the final wash step being a 1:10 dilution.

### Example 12: HTS Viable Cell Enrichment—Single Step Workflow

All wells of a 96-well plate were loaded with Jurkat cells with a starting viability of 50% in levitation medium containing 150 mm gadobutrol. The samples were enriched using a single-step Levitation-based viable cell enrichment protocol wherein the plate was placed in the levitation array in a Levicell system, and the samples levitated to their equilibrium positions. Live cells levitate at a different height than dead cells. A pipette was used to retrieve the live cells from the well and transfer them to an output well. The mean viability of the transferred sample was measured to be 89%. This indicates the successful enrichment of viable cells using the technology.

### Example 13: CD45 Depletion

All wells of The LeviSelect CD45 depletion kit was used to remove CD45+ cells from a mixture of 85% Jurkat (CD45 pos) and 15% H358 (CD45 neg) cells and levitated on the HTS platform. Jurkat (CD45+) cells were stained with Calcein and H358 (CD45−) cells were stained with celltracker red (CTR) and then mixed: 85% Jurkat and 15% H358. A standard depletion reaction on 1e6 total cells/270 ul, and 50 ul was aliquoted into wells of the LV array as described herein. A “concentrated” depletion on 5e5 cells/50 ul was also conducted similarly. Measurements were made of 1) the depletion rate—i.e., the number of Jurkat cells in the output/the number of Jurkat cells in the input: 2) the yield—i.e., the number of H358 cells in the output/the number of H358 cells in the input; and 3) the total viability in the input and output. Both the conventional and “concentrated” methods worked well to deplete CD45+ cells on the Ruby system, with a yield of >than 24%. The results showed that the CD45 positive cells were effectively removed leaving a pure sample of CD45 negative cells.

### Example 14: Isolation of Human Brain Nuclei with LeviCell Using 3 Micron Streptavidin Coated Polystyrene Beads Using a Well-Based System

System 1 is used to separate human brain nuclei from a preparation containing the nuclei and both large and small cellular debris. Human brain nuclei are isolated and prestained with propidium iodide in a wash buffer, and then resuspended in PBS/BSA/propidium iodide. 3-3.4 micron streptavidin coated polystyrene beads (SVP-30-5, Spherotech, Inc., Lake Forest IL) are then added to a concentration of approximately 130 beads/nuclei. Controls with no beads are also prepared. Gadolinium levitation buffer is added to the samples to provide 150 mM gadolinium, and the samples are loaded into the wells of a 96-well plate. The samples are enriched using a single-step Levitation-based nuclei enrichment protocol wherein the plate was placed in the levitation array in a Levicell system, and the samples levitated to their equilibrium positions. Isolated nuclei levitate at a different height than dead cells and other debris. A pipette is used to retrieve the isolated nuclei from the well and transfer them to an output well.

### Example 15—Isolation of Human Brain Nuclei with LeviCell Using 3 Micron Streptavidin Coated Polystyrene Beads and RNA Sequencing Using a Well-Based System

System 1 is used as described above to separate human brain nuclei, which is then subjected to RNA sequencing.

Nuclei are isolated from Human brain tissue according to the following procedure. Tissue is minced and homogenized in nuclei isolation buffer with and without WGA. The samples are filtered through 70 um sieve w/PBS/BSA/RNAseOUT, and washed (centrifugation) with 1×/PBS/BSA/RNAseOUT, and resuspended in PBS/BSA/RNAseOUT.

For Set 1, two pools are made: 1 (5rxn equivalent w/PI) and 1 (5rxn equivalent NO PI) 250 ul each, to which was added 375 ul of PBS/BSA/Pi or PBS/BSA/rnase inhibitor to 625 ul. The pools are divided into pools of 125 ul each, to which is added 80 ul bead (˜165 bead/nuclei) bead 5 minutes at room temperature or 80 ul PBS as control. 70 ul of levitation buffer is added to achieve a final concentration of 150 mM Gd. The samples are loaded into the wells of a 96-well plate in 150 mM, Gd 1% Pi, and the nuclei yields and purity of bottom fractions are measured.

For Set 2, to 50 ul of each preparation is added 75 ul PBS/BSA/rnase inhibitor, then 80 ul bead (˜100˜130 bead/nuclei) bead 5 minutes at room temperature, or 80 ul PBS as control. 70 ul of levitation buffer is added to achieve a final concentration of 150 mM Gd. The samples are loaded into the wells of a 96-well plate in 150 mM, Gd 1% Pi, and the nuclei yields and purity of bottom fractions are measured. Parallel runs are also performed without propidium iodide for use in subsequent RNA sequencing using a 3′ RNA LT kit. Target˜700 nuclei/sample.

### Results

The sample which has beads and uses WGA in the nuclei preparation, will be expected to have both a high yield and a high purity.

Results of RNA sequencing: The RNA in the samples are sequenced using a 10×3′ RNA LT kit, Target˜700 nuclei/sample. The samples which have both WGA and the beads present, will be expected to have less mitochondrial reads compared to any of the other experiments. It is also expected that there also will be less ribosomal RNA in these preparations, which in a nuclear prep is also a cytoplasmic contaminant.

### IX. Disclosed Embodiments are Non-Limiting

While various embodiments of the present invention have been shown and described herein, it is emphasized that such embodiments are provided by way of example only. Numerous variations, changes and substitutions may be made without departing from the invention herein in its various embodiments. Specifically, when any range is described herein, unless clearly stated otherwise, that range includes all values therein and all subranges therein.

Also, and more generally, in accordance with disclosures, discussions, examples and embodiments herein, there may be employed conventional fluidics, molecular biology, cellular biology, microbiology, and recombinant DNA techniques within the skill of the art. Resources incorporated by reference herein are for their respective content and teachings found therein. Such incorporation, at a minimum, is for the specific teaching and/or other purpose that may be noted when citing the reference herein. If a specific teaching and/or other purpose is not so noted, then the published resource is specifically incorporated for the teaching(s) indicated by one or more of the title, abstract, and/or summary of the reference. If no such specifically identified teaching and/or other purpose may be so relevant, then the published resource is incorporated in order to more fully describe the state of the art to which the present invention pertains, and/or to provide such teachings as are generally known to those skilled in the art, as may be applicable. However, it is specifically stated that a citation of a published resource herein shall not be construed as an admission that such is prior art to the present invention. Also, in the event that one or more of the incorporated published resources differs from or contradicts this application, including but not limited to defined terms, term usage, described techniques, or the like, this application controls as a preferred embodiment, and any contradiction may be viewed as an alternative embodiment.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

1. A method for extracting cellular nuclei from intact cells comprising;
providing intact cells; and
lysing the cells in a nuclei isolating buffer;
wherein the nuclei isolating buffer comprises wheat germ agglutinin (WGA),
optionally wherein the nuclei are isolated from healthy cells, diseased cells, infected cells, transfected cells, or genetically modified cells;
optionally wherein the method further comprises;
collecting the nuclei, e.g. by centrifugation; and
optionally storing the nuclei; for example by suspending the nuclei in a storage buffer; for example wherein the storage buffer comprises sucrose; optionally wherein the WGA is present in an amount of from about 0.01 mg/mL to about 2 mg/mL; e.g., from about 0.01 mg/mL to about 1 mg/mL; e.g., from about 0.01 mg/mL to about 0.5 mg/mL; e.g., 0.05 mg/mL to about 0.15 mg/mL; e.g., about 0.1 mg/mL;
and the nuclei isolating buffer comprises said WGA and one or more of buffers, solvents, proteins, particles, enzymes, and stabilizers; and water.

2-4. (canceled)

5. The method according to claim 1 wherein the integrity of isolated cell nuclei is at least about 15% greater than the integrity of cell nuclei isolated by a similar method lacking WGA; and/or the method further comprises suspending the collected nuclei in a levitation buffer containing a levitation agent; for example wherein the levitation agent comprises 100 mM Gadolinium, and the levitation buffer comprises 1×PBS, 1% BSA, and a RNAse inhibitor, e.g. RNAseOUT™.

6-7. (canceled)

8. A method of isolation of a target subcellular component, e.g. cellular nuclei, from a sample comprising the target subcellular component and one or more contaminating species, comprising:
loading a sample comprising the target subcellular component, the contaminating species, and a sample medium comprising:
i) a paramagnetic compound or ferrofluid; and
ii) isolation particles (or beads);
into a well or separation channel along which the sample is optionally caused to flow;
subjecting the sample to a magnetic force with at least one magnet to affect a separation of the target subcellular component from other components of the sample;
collecting at least one fraction of the separated sample comprising the target subcellular component without further centrifugation and;
optionally imaging the target subcellular component in the sample prior to, during, and/or after the separation;
wherein the isolation particles are from about 10 nanometers to about 10 microns in size; e.g. about 1 to about 8 microns in size; e.g. about 1 to about 5 microns in size; e.g. about 1, about 2, about 3, about 4 or about 5 microns in size; or from about 10 nanometers to about 1 micron in size; e.g. about 10 to about 800 nanometers in size; e.g. about 10 to about 500 nanometers in size; e.g. about 100, about 200, about 250, about 300, about 350, about 400, about 450, or about 500 nanometers in size; and wherein:
a) the isolation particles form a complex with one or more of the contaminating species, or form a structure in the sample medium that interacts with one or more of the contaminating species, in a manner that inhibits the movement of the one or more contaminating species in a chosen direction relative to the movement of the target subcellular component in the same direction; or
b) the isolation particles form a complex with the one or more of the contaminating species, or form a structure in the sample medium that interacts with one or more of the contaminating species, in a manner to increase the movement of the one or more contaminating species in a chosen direction relative to the movement of the target subcellular component in the same direction;
optionally wherein the cell nuclei are isolated from human cells, non-human animal cells, or plant cells.

9. The method according to claim 8, wherein the particles form a complex with one or more of the contaminating species, or form a structure in the sample medium that interacts with one or more of the contaminating species, in a manner that inhibits the movement of the one or more contaminating species in the direction of ambient gravitational force relative to the movement of the target subcellular component in the same direction.

10-12. (canceled)

13. The method according to claim 8, wherein the isolation particles are selected from agarose beads, e.g. Sepharose™ beads, dextran beads e.g. Sephadex™, polyacrylamide beads, dextrose beads, polystyrene beads, beads made from polymeric resins. e.g. polyvinylethylcarbitol, polyvinylpyrrolidone, cellulose, silica-based materials, and/or from mixtures such as dextran-polyacrylamide, e.g. Sephacryl™ beads; each of which can optionally be coated, for example with a protein; e.g. streptavidin coated polystyrene beads or particles; and
wherein the isolation particles have a size of about 3 microns; or from about 10 nanometers to about 1 micron in size; e.g. about 10 to about 800 nanometers in size; e.g. about 10 to about 500 nanometers in size; e.g. about 100, about 200, about 250, about 300, about 350, about 400, about 450, or about 500 nanometers in size.

14-16. (canceled)

17. The method according to claim 8 wherein the sample comprises from about 50 to about 10,000,000 cell nuclei; and/or
wherein the concentration of cell nuclei in a fraction is increased by at least 1.1:1 from the original sample; and/or
wherein the concentration of non-nuclei particles in the original sample is decreased by at least about 1% in the fraction; and/or
wherein the integrity of isolated cell nuclei in a fraction from a sample is at least about 30% greater than the integrity of cell nuclei isolated in a fraction from a sample by a method comprising centrifugation.

18-44. (canceled)

45. A kit for isolating cell nuclei comprising:
a) a nuclei isolation buffer and one or more additional buffers, solvents, proteins, particles, enzymes, and/or stabilizers;
wherein the isolation particles comprise beads, for example polystyrene beads, optionally coated with a protein, e.g. streptavidin, having a size of from about 10 nanometers to about 10 microns in size; e.g. about 1 to about 8 microns in size; e.g. about 1 to about 5 microns in size; e.g. about 1, about 2, about 3, about 4 or about 5 microns in size; or from about 10 nanometers to about 1 micron in size; e.g. about 10 to about 800 nanometers in size; e.g. about 10 to about 500 nanometers in size; e.g. about 100, about 200, about 250, about 300, about 350, about 400, about 450, or about 500 nanometers in size.

46-48. (canceled)

49. A kit of claim 45, wherein one or more of the components of the kit are provided as solids for reconstitution; further comprising a levitation agent comprising gadolinium.

50. (canceled)

51. The method of claim 8, wherein the method is performed in a well.

52. The method of claim 8, wherein the method is performed utilizing a system for conducting magnetic levitation separation of samples in a multi-well plate, the system comprising:
a levitation sample fixture comprising:
a multi-well plate comprising a top surface and a plurality of wells, wherein said wells are optionally optically transparent;
a magnet array comprising a plurality of magnets disposed in between and below the wells, configured to provide a magnetic field in each of the wells;
a magnet holder configured to receive and hold the magnets of the magnet array;
optionally, a mirror assembly comprising:
mirrors positioned to project, substantially parallel to the top surface of the plate, images of the wells along their vertical axes; and
optionally a mirror holder;
optionally, a plurality of metal pins configured to attenuate and/or adjust the magnetic fields in the wells; and
optionally, an imaging array disposed beneath the multi-well plate and magnet array;
optionally wherein:
the wells are conical; or
the wells have four vertical sides and a square bottom;
the method optionally further comprising removing the multi-well plate and replacing it with another multi-well plate; optionally wherein:
the contaminating species is a dye or dyes; and/or
the visualization system is used to adjust the parameters for transferring the sample into or out of the well.

53. The method of claim 52, wherein the system further comprises a fluid transfer system (e.g., means to dispense or remove all or part of a sample or other fluid into or out of the wells; e.g., a pipetting robot) and/or
means to move the multi-well plate in a vertical direction relative to the magnet array.

54. (canceled)

55. The method of claim 52, wherein the magnet array comprises permanent magnets; for example wherein, for each well:
a first magnet and a second magnet are disposed adjacent to the well to impart a magnetic field that is inside the well; wherein each magnet comprises a first pole and a second pole, and the first pole of the first magnet faces the first pole of the second magnet; and
a third magnet is stacked under the first magnet, and a fourth magnet is stacked under the second magnet, wherein the third and fourth magnets each comprise a first pole and a second pole, and wherein the second pole of the third magnet faces the second pole of the fourth magnet;
or
a first magnet and a second magnet are disposed adjacent to, and on opposite sides of, the well to impart a magnetic field that is inside the well; wherein each magnet comprises a first pole and a second pole, and the first pole of the first magnet faces the first pole of the second magnet; a third magnet is stacked under the first magnet, and a fourth magnet is stacked under the second magnet, wherein the third and fourth magnets each comprise a first pole and a second pole, and wherein the second pole of the third magnet faces the second pole of the fourth magnet; a fifth magnet and a sixth magnet are disposed adjacent to, and on opposite sides of, the well, to impart a magnetic field that is inside the well;
wherein each magnet comprises a first pole and a second pole, and the first pole of the fifth magnet faces the first pole of the sixth magnet, and wherein the fifth and sixth magnets are oriented at approximately 90 degrees in the horizontal plane relative to the first and second magnets; and
a seventh magnet is stacked under the fifth magnet, and an eighth magnet is stacked under the sixth magnet, wherein the seventh and eighth magnets each comprise a first pole and a second pole, and wherein the second pole of the seventh magnet faces the second pole of the eighth magnet;
optionally wherein the magnets are rectangular magnets configured substantially in accordance with FIG. 35 or FIG. 40B;
or
wherein the magnets are arranged in a linear Halbach array.

56-58. (canceled)

59. The method of claim 55, wherein the magnets are ring magnets that surround the wells;
optionally wherein the magnets are radially magnetized;
optionally wherein the system comprises for each well two ring magnets stacked one upon the other, wherein the poles of the stacked magnets are radially opposed;
wherein the stacked ring magnets are configured to create a strong gradient at the interface with a low field zone in the height of the upper magnet;
optionally wherein the magnets are configured to be axially magnetized.

60-63. (canceled)

64. The method of claim 52, wherein the magnets provide a magnetic field in each of the wells of between about 0.1 Tesla and about 2.0 Tesla and optionally between about 0.3 Tesla and about 1.0 Tesla, at the surfaces of the magnets, varying to zero Tesla in certain locations due to superposition of fields from the plurality of magnets.

65. The method of claim 52, wherein the imaging array is present and comprises:
a) a microscope, e.g., a USB microscope and means for moving the microscope underneath the multi-well plate from well to well; or
b) a camera array comprising a plurality of cameras; for example a motorized camera array; optionally wherein the plurality of cameras comprise one or more of built-in lenses, motorized focus, and zoom-in capability; and wherein each camera is configured to capture images from one to four of the wells; for example from two to three of the wells;
wherein the system optionally further comprises a graphics processing unit (GPU) comprising graphics software that integrates two or more of the images from the cameras.

66-67. (canceled)

68. The method of claim 52, wherein the mirror assembly is present and comprises a mirror holder and mirrors; optionally wherein the mirrors are either:
a) a separate layer with reflective surfaces; or
b) a mirror-coating disposed on the mirror holder;
optionally wherein the mirrors are positioned at about 40° to about 50° with respect to the vertical axis substantially perpendicular to the main surface of the multi-well plate;
optionally wherein the mirrors are configured substantially in accordance with FIG. 2.

69-73. (canceled)

74. The method of claim 52, wherein:
A: the levitation sample fixture comprises a plurality of metal pins configured to attenuate and/or adjust the shape of the magnetic fields in the wells; optionally wherein the metal pins are steel rods disposed in between the wells, either linearly or diagonally between the wells, and are configured to increase the magnetic field gradients within the wells, e.g., within the walls of the wells, or at preselected area(s) within the wells; optionally wherein the metal pins are disposed substantially as shown in FIG. 60B; and/or optionally wherein the metal pins are steel, manganese alloy, nickel alloy or chromium alloy rods;
and/or
B: the multi-well plate is configured to fit in the footprint of a standard 96-well PCR plate, or is in accordance with ANSI SLAS microplate standards; e.g., with a footprint of 127.76 mm×85.48 mm+0.5 mm.

75-6. (canceled)

77. A method for separating nuclei from a sample comprising said nuclei and one or more contaminating species, the method comprising:
loading a) the sample; and b) a paramagnetic medium comprising a paramagnetic compound or ferrofluid, into a well of a system comprising:
a levitation sample fixture comprising:
a multi-well plate comprising a top surface and a plurality of wells, wherein said wells are optionally optically transparent;
a magnet array comprising a plurality of magnets disposed in between and below the wells, configured to provide a magnetic field in each of the wells;
a magnet holder configured to receive and hold the magnets of the magnet array;
optionally, a mirror assembly comprising:
mirrors positioned to project, substantially parallel to the top surface of the plate, images of the wells along their vertical axes; and
optionally a mirror holder;
optionally, a plurality of metal pins configured to attenuate and/or adjust the magnetic fields in the wells; and
optionally, an imaging array disposed beneath the multi-well plate and magnet array to form a sample fluid or sample suspension in said well; and
subjecting the sample fluid or sample suspension to a magnetic force from at least one magnet from the magnet array, to effect separation of the live cells from the contaminating species; and
optionally imaging the sample fluid or sample suspension prior to, during, and/or after the separation; and
optionally collecting the separated nuclei;
optionally wherein the contaminating species is selected from one or more of a dissolved or suspended compound (e.g., a dye or dyes, antibodies, etc.); cellular debris; small particles (e.g., micro- or nano-beads); and dead cells;
optionally wherein the paramagnetic medium comprises one or more of a paramagnetic salt, a paramagnetic hydrophobic metal chelate, a paramagnetic metal chelate, or a paramagnetic ionic liquid.

78-84. (canceled)

85. The method of claim 82, wherein the separated nuclei are collected by the steps of:
A:
a) moving the multi-well plate vertically upward relative to the magnet array while maintaining the separated nuclei in place with the magnetic field, thereby causing the separated nuclei to migrate to the bottom of the well; and
b) either:
i) removing the liquid above the separated nuclei with the pipette tip, leaving the separated nuclei in a reduced volume of fluid at the bottom of the well; or
ii) removing the separated nuclei from the bottom of the well with the pipette tip;
or by the steps of B:
Ba1) moving the pipette tip a vertical direction while maintaining the separated nuclei in place with the magnetic field, to bring the nuclei towards the top of the fluid in the pipette tip; and
Ba2) dispensing the liquid below the nuclei while retaining the separated nuclei in the pipette tip;
or
Bb1) moving the pipette tip a vertical direction while maintaining the separated nuclei in place with the magnetic field, to bring the separated nuclei to the bottom of the pipette tip; and
Bb2) selectively dispensing the separated nuclei, leaving unwanted liquid in the pipette tip;
or by the steps of C:
Ca) inserting the pipette tip into the sample past the levitation position of the separated nuclei;
Cb) drawing the liquid up into the pipette tip while maintaining the separated nuclei in place with the magnetic field; and
Cc) dispensing just the separated nuclei;
or by the steps of D:
Da) after achieving levitation equilibrium, aspirating the liquid into the pipette tip until the separated live cells are aspirated;
Db) withdrawing the pipette tip from the well and immersing it into a second well in the plate or a separate plate that contains a washing liquid;
Dc) aspirating the separated nuclei into the washing liquid while retaining the liquid in the pipette; i.e., slowly removing the pipette tip and the liquid therein, while the field maintains the position of the levitated nuclei, thus separating them from the original liquid; and
optionally repeating steps (Da)-(Dc); optionally wherein step (Da) is performed early in the levitation process;
or by the steps of E:
Ea) after achieving levitation equilibrium, aspirating the liquid into the pipette tip until the separated live cells are aspirated;
Eb) withdrawing the pipette tip from the well and immersing it into a second well in the plate or a separate plate that contains a washing liquid;
Ec) aspirating the separated nuclei into the washing liquid while retaining the liquid in the pipette; i.e., slowly removing the pipette tip and the liquid therein, while the field maintains the position of the levitated nuclei, thus separating them from the original liquid; and
optionally repeating steps (Ea)-(Ec).

86-89. (canceled)

90. The method of claim 85, wherein the nuclei are levitated by radial magnets, and collected by the steps of:
a) aspirating the entire sample into the pipette tip;
b) moving the pipette tip and sample vertically upwards until they are clear of, and above, the magnets;
c) slowly lowering the tip into the magnetic field, and allowing a spheroid of particles to begin to coalesce in the region of minimal field;
d) after the tip completes passage through the region of minimal field, ejecting most of the tip's contents while the tip is moved slowly upwards, leaving a concentrated sample at the bottom of the tip; and
e) optionally transferring the concentrated sample;
or by the steps of:
a1) slowly aspirating the entire sample into the pipette tip creating a spheroid of particles at the region of minimal field;
b1) moving the pipette tip vertically upwards through the magnetic field while the particle spheroid remains stationary relative to the field;
c1) rapidly moving the sample to a destination, e.g., an output well; and
d1) depositing a small volume containing the spheroid in the destination.

91. (canceled)

92. A method for enriching a population of interest, said population of interest comprising nuclei, the method comprising:
loading, into a well of a system comprising:
a levitation sample fixture comprising:
a multi-well plate comprising a top surface and a plurality of wells, wherein said wells are optionally optically transparent;
a magnet array comprising a plurality of magnets disposed in between and below the wells, configured to provide a magnetic field in each of the wells;
a magnet holder configured to receive and hold the magnets of the magnet array;
optionally, a mirror assembly comprising:
mirrors positioned to project, substantially parallel to the top surface of the plate, images of the wells along their vertical axes; and
optionally a mirror holder;
optionally, a plurality of metal pins configured to attenuate and/or adjust the magnetic fields in the wells; and
optionally, an imaging array disposed beneath the multi-well plate and magnet array, individually or as a pre-mix, a) a sample comprising the population of interest; and b) a paramagnetic medium comprising a paramagnetic compound or ferrofluid, to form a sample fluid or sample suspension in said well; and
subjecting the sample fluid or sample suspension to a magnetic force from at least one magnet from the magnet array, to effect enrichment of the population of interest; and
optionally imaging the sample fluid or sample suspension prior to, during, and/or after the enrichment;
optionally wherein the paramagnetic medium comprises one or more of a paramagnetic salt, a paramagnetic metal chelate, or a paramagnetic ionic liquid; for example a water soluble paramagnetic metal chelate; and wherein the method optionally further comprises collecting the population of interest; optionally wherein the separated population of interest is collected from the well in a pipette tip.

93-5. (canceled)

96. The method of claim 92 wherein the separated population of interest is collected by the steps of:
A:
a) moving the multi-well plate vertically upward while maintaining the separated population of interest in place with the magnetic field, thereby causing the cells to migrate to the bottom of the well; and
b) either:
i) removing the liquid above the separated population of interest with the pipette tip, leaving the separated population of interest in a reduced volume of fluid at the bottom of the well; or
ii) removing the separated population of interest from the bottom of the well with the pipette tip;
or by the steps of B:
Ba1) moving the pipette tip a vertical direction while maintaining the separated population of interest in place with the magnetic field, to bring the separated population of interest to the top of the pipette tip; and
Ba2) dispensing the liquid below the separated population of interest while retaining the separated cells in the pipette tip;
or:
Bb1) moving the pipette tip a vertical direction while maintaining the separated population of interest in place with the magnetic field, to bring the cells to the bottom of the pipette tip; and
Bb2) selectively dispensing the separated population of interest, leaving unwanted liquid in the pipette tip;
or by the steps of C:
Ca) inserting the pipette tip into the sample past the levitation position of the separated population of interest;
Cb) drawing the liquid up into the pipette tip while maintaining the separated population of interest in place with the magnetic field; and
Cc) dispensing just the separated population of interest;
optionally wherein step Ca is performed early in the levitation process;
or by the steps of D:
Da) after achieving levitation equilibrium, aspirating the liquid into the pipette tip until the separated population of interest is aspirated;
Db) withdrawing the pipette tip from the well and immersing it into a second well in the plate or a separate plate that contains a washing liquid;
Dc) aspirating the separated population of interest into the washing liquid while retaining the liquid in the pipette; and
optionally repeating steps (a)-(c).

97-100. (canceled)

101. The method of claim 52, wherein:
the magnetic field strength at a surface of at least one of the magnets adjacent to the wells is between about 0.1 Tesla and about 2.0 Tesla and optionally between about 0.3 Tesla and about 1.0 Tesla; and/or
the paramagnetic compound is present in the sample solution at a concentration of from about 20 mM to about 500 mM, optionally from about 50 mM to about 175 mM, and further optionally from about 70 mM to about 150 mM; and/or
the enriched recovered sample comprises at least about 60%, at least about 70%, at least about 80% or at least about 90% nuclei; and/or
the yield of nuclei in the enriched recovered sample fraction is at least about 50%, at least about 60%, at least about 70%, or at least about 75% of the total nuclei composition of the sample; and/or
the sample is loaded into the well or wells, and/or transferred out of the well or wells, using a pipetting robot; and/or
the magnetic levitation is monitored and/or recorded in real time by the visualization system.

102-111. (canceled)

112. The method of claim 1, wherein the method is performed in a well.
